(19) United States
(12) Patent Application Publication
(10) Pub. No.: US 2009/0142429 A1
(43) Pub. Date: Jun. 4, 2009

(54) SILICIBACTER SP. STRAIN USEFUL FOR GENETIC TRANSFORMATION OF MARINE ALGAE AND PRODUCTION OF ANTIBIOTIC AGENTS

(75) Inventors: Robert Belas, Catonsville, MD (US); Todd R. Miller, Janesville, WI (US); Jesper Bartholin Bruhn, Soeborg (DK)

Correspondence Address: INTELLECTUAL PROPERTY / TECHNOLOGY LAW, PO BOX 14329, RESEARCH TRIANGLE PARK, NC, 27709, US

(73) Assignee: UNIVERSITY OF MARYLAND BIOTECHNOLOGY INSTITUTE, Baltimore, MD (US)

(21) Appl. No.: 11/915,331

(22) PCT Filed: May 23, 2006
(86) PCT No.: PCT/US06/20103

§ 371 Date: Mar. 28, 2008

Related U.S. Application Data
(60) Provisional application No. 60/683,763, filed on May 23, 2005.
(60) Provisional application No. 60/718,542, filed on Sep. 19, 2005.

(51) Int. Cl. A61K 35/00; C12N 1/20; C12N 1/13; A61P 31/04; C12N 15/74; C12P 21/00

(52) U.S. Cl. 424/780; 435/252.1; 435/257.2; 435/471; 435/69.1

(57) ABSTRACT

A Silicibacter sp. strain useful for genetic transformation of marine algae and production of antibiotic agents is described. Silicibacter sp. TM1040 is a genetically tractable member of the marine Roseobacter clade of bacteria that forms an intimate and obligate interaction with algae, and is useful as a probiotic for producing antibacterial agents that are cidally effective against pathogenic bacteria such as Mycobacterium marinum, Vibrio anguillarum, V. coralliilyticus, and V. shiloi bacteria.

### CROSS-REFERENCE TO RELATED APPLICATIONS

This application claims the benefit of priority of U.S. Provisional Patent Application No. 60/683,763 filed May 23, 2005 in the names of Robert Belas, et al. for “Discovery of a Novel Silicibacter Sp. Strain and Use Thereof for Genetic Transformation of Marine Algae,” and the benefit of priority of U.S. Provisional Patent Application No. 60/718,542 filed Sep. 19, 2005 in the names of Robert Belas, et al. for “Silicibacter Sp. TM1040 Antibacterial Compound Production and Use Thereof.” The disclosures of said U.S. Provisional Patent Application No. 60/683,763 and U.S. Provisional Patent Application No. 60/718,542 are hereby incorporated herein by reference in their respective entireties.

### BACKGROUND OF THE INVENTION

1. Field of the Invention

The present invention relates to a Silicibacter sp. strain, and more particularly, to Silicibacter sp. TM1040 a genetically tractable member of the marine Roseobacter clade of bacteria that forms an intimate and obligate interaction with algae and use of said bacteria for genetically transforming said algae, as well as use of such bacteria as a probiotic agent, alone or in compositions, and antibiotic compounds and compositions derived from such bacteria.

2. Description of the Related Art

Pfiesteria piscicida, Pfiesteria shumwayae, and Pfiesteria-like organisms, such as Cryptoperidiniopsis sp., are estuarine, heterotrophic dinoflagellates with a global distribution. Reports have implicated Pfiesteria sp. as the causative agent of massive fish deaths along the Atlantic Coast of the United States, especially in the estuaries of Pamlico Sound, N.C., and the Chesapeake Bay of Maryland and Virginia (Burkholder et al., 1992). While the toxicity of Pfiesteria species is an important question, other physiological aspects of these dinoflagellates deserve further attention.

In the marine environment, these dinoflagellates coexist with a diverse community of heterotrophic bacteria. Much of the carbon and nitrogen required for the growth of these bacteria is supplied by the unicellular bloom-forming eukaryotic algae (Cole et al., 1982). During feeding, stress, and lysis, nutrients are released from algae and made available to the bacteria. The nutrient plumes emitted occur on a microscale and are quickly dispersed and diluted by diffusion and turbulence.

Not all species of bacteria are equally likely to obtain these nutrients (Blackburn et al., 1998). The α-, β-, and γ-proteobacteria, as well as the Cytophaga-Flexibacter-Bacteroides spp., are important members of the marine picoplankton and are often found within algal communities (Giovannoni et al., 2000). Of these groups, the α-Proteobacteria genera phylogenetically related to the Roseobacter clade are of particular interest. Bacteria phylogenetically related to Roseobacter clade are an abundant group of marine bacteria that are associated with algae in both laboratory and field studies. Although many of the bacterial taxa abundant in the marine environments have never been cultured, the Roseobacter clade is an exception and can be readily cultured in the laboratory. They are heterotrophic, Gram-negative, rods, ca. 1.5-2.0 by 0.5-0.75 μm. The majority of its genera are from marine habitats and have a strict requirement for NaCl. Most genera are motile via one or more flagella. Furthermore, some Roseobacter species exhibit close physical (attached and intracellular) or physiological (growth-enhancing) relationships with DMSP-producing dinoflagellates, including Prorocentrum, Alexandrium, and Pfiesteria species.

In marine surface waters, α-proteobacteria phylogenetically related to Roseobacter spp. are predominantly responsible for the degradation of DMSP, its catabolites, and other sulfonium compounds (Gonzalez et al., 1999). Although Roseobacter spp. are cosmopolitan in nature, their production and activity are significantly correlated with DMSP-producing algae, including dinoflagellates and prymnesiophytes (Gonzalez et al., 1997; Zubkov et al., 2001). Furthermore, some Roseobacter spp. exhibit close physical or physiological relationships with toxic, DMSP-producing dinoflagellates, including Pfiesteria spp. (Alavi et al., 2001) where these bacteria can be found physically attached to and aiding in the growth of the dinoflagellates.

Dimethylsulfoniopropionate (DMSP) is the major source of organic sulfur in the world's oceans and plays a significant role in the global sulfur cycle (Yoch et al., 1997). During blooms of marine unicellular algae, cellular DMSP is released due to algal senescence, predation, or stress and is degraded by both algal and bacterial enzymes. In marine environments, dinoflagellates and prymnesiophytes are the major producers of DMSP, with intracellular concentrations as high as 0.5 M.

Bacteria and certain species of phytoplankton produce an enzyme, dimethylpropiothetin dethiomethylase (DMSP lyase) (EC 4.4.1.3), which degrades DMSP to produce dimethylsulfide (DMS) and acrylate (Yoch et al., 1997). Acrylate is readily consumed by bacteria and is converted to β-hydroxypropionate by α- and β-proteobacterial species (Ansede et al. 1997 and 2001). Bacteria may also demethylate DMSP at the DMS moiety, producing 3-methylmercaptopropionate (MMPA), which may be further demethylated to 3-mercaptopropionate MPA or demethiolated to produce acrylate and methanethiol (MeSH). The products of these reactions provide a rich source of carbon and sulfur for bacterial production (Kiene et al., 1996).

While some bacterial species rely upon high-affinity uptake mechanisms, the ability to sense and move towards increasing chemical gradients, known as chemotaxis, provides motile bacteria with a distinct advantage over their nonmotile counterparts (Blackburn et al., 2001). Some Roseobacter species have developed close associations with dinoflagellates and phytoplankton and these biological interactions between dinoflagellates and bacteria may influence both the rate of primary production and the fate of fixed carbon in the surface ocean.

Knowledge of how marine bacteria sense and respond to algal cells is therefore important to the understanding of bacterial physiology and the interactions between these prokaryotes and their eukaryotic hosts and ultimately impacts the greater understanding of nutrient cycling in marine ecosystems. Mechanisms for these interactions may rely upon bacterial motility and chemotaxis behavior that allow these bacteria to sense and move towards their eukaryotic host, where they derive beneficial nutrients, once in close physical proximity to the dinoflagellate.

Thus, it would be of great benefit to isolate native bacteria that coexist and interact with dinoflagellates to determine how these bacteria assimilate dinoflagellate-derived nutrients and are intrinsically propagated within the ecological environment with the dinoflagellates. Further, it would be of great benefit to discover native bacteria that can transfer gene material to the dinoflagellates for expression therein.

Recent studies have demonstrated that probiotic bacteria may be used to control pathogenic organisms in fish larval rearing (12, 26), and one of several promising candidates is bacteria of the marine Roseobacter clade. The ability of Roseobacter to inhibit other bacteria was first noted by Ruiz Ponte et al. (25) who used Roseobacter gallaeciensis strain BS107 as a probiotic treatment of scallop larvae (26). Roseobacter strains have also been isolated from turbot larval farms where they were selected from this environment due to their strong anti-Vibrio activity (9, 12, 13).

Brinkhoff et al. (8, 15) recently demonstrated that the inhibitory compound produced by the Roseobacter strain T5 is the sulfur-containing molecule, tropodithietic acid. This is a precursor of the antibacterial compound thiotropocin isolated from a marine isolate of Agrobacterium (27), a species now recognized as a member of the Roseobacter clade (28). Another strain, Roseobacter strain 27-4, also produces thiotropocin or tropodithietic acid (their chemical similarity makes it difficult to distinguish between the two), which constitutes as much as 15% of metabolite production (9).

Several environmental factors affect tropodithietic acid production. Production of tropodithietic acid is correlated with a brownish pigment since non-pigmented (mutant) colonies are devoid of antibacterial activity (8, 9). However, the brownish pigment is not the primary bioactive compound as the full bioactivity of the extract was found in fractions and the pigment was not eluted from the column. It is also known that thiotropocin or tropodithietic acid is stable at different pH, but incubation temperature influences the activity, which declines rapidly with increasing growth temperature. The antibacterial activity is very sensitive to culture aeration, since the antibacterial compound is only found under static growth conditions. Static growth conditions also cause roseobacters to grow as multicellular rosettes (star-shaped aggregations of bacteria) (9, 14, 24, 28). Thus, tropodithietic acid production is positively correlated with static growth in marine broth at temperatures between 15-24° C. and is associated with the formation of a brown pigment and rosettes of cells.

The dramatic growth in the aquaculture sector (an increment of 9-10% per year over the last 8-10 years) has emphasized the importance of disease control in all ages of animal husbandry from larva to adult. Bacterial diseases are important constraints, especially in recirculating aquaculture systems, and may be treated with antibiotics. However, due to the risk of development and transfer of antibiotic resistance, alternative disease control measures must be implemented. Vaccines have been very successful also in fish farming, however, vaccines are not efficient at the larval stages. “Greenwater” (unicellular algae plus their associated microbial flora) is routinely used as a probiotic in recirculating aquaculture systems. Several studies have demonstrated that probiotic bacteria may be used to control pathogenic organisms in fish larval rearing and in adults (12, 26). Therefore, since Roseobacter often physically attach to algae and dinoflagellates (10, 11, 16), there is a potential to use these bacteria, either by themselves or together with greenwater, as a probiotic in the prevention of diseases of finfish and shellfish.

Thus, it would be of great benefit to isolate native bacteria that have a potent antibacterial activity that is effective in killing pathogenic bacteria not only in fish but also mammals.

### SUMMARY OF THE INVENTION

In one aspect, the present invention provides for an isolated Silicibacter sp. microorganism for interaction with the estuarine dinoflagellate Pfiesteria piscicida and increasing growth of same, the Silicibacter sp. comprising a 16S ribosomal subunit nucleic acid sequence selected from the group consisting of:
(a) a nucleic acid sequence that has more than 95% identity to a nucleic acid sequence of SEQ ID NO 2; and
(b) a nucleic acid sequence fully complementary to a nucleic acid of (a); and
wherein the isolated microorganism interacts with marine algae to modify growth thereof.

In another aspect, the present invention relate to delivery device for delivering a nucleotide sequence encoding a protein of choice for expression by an algae, the method comprising a Silicibacter sp. microorganism comprising SEQ ID NO: 1.

In another aspect, the present invention provides for a newly discovered strain of Silicibacter sp. comprising a 16S nucleotide sequence. (SEQ ID NO: 2)

In yet another aspect, the present invention relates to an algae cell transfected with a DNA or RNA comprising at least one nucleotide sequence selected from the group consisting of SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13 and SEQ ID NO. 14.

In another aspect the present invention relates to a protein of choice production system, comprising algae transfected with a recombinant Silicibacter sp. strain, wherein the Silicibacter sp. strain comprises a nucleotide sequence encoding for the protein of choice and at least one nucleotide sequence selected from the group consisting of SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, and SEQ ID NO. 14.

In another aspect, the present invention relates to a delivery vector for delivery genes from a bacterium to a plant cells, the delivery vector comprising a Silicibacter sp. strain comprising: vir genes whose function is involved in the transfer of DNA from the bacterium to plant cells. Preferably, the Silicibacter sp. TM1040 strain is used to deliver genes encoding a protein of choice to marine algal species.

In yet another aspect, the present invention relates to a method for delivering a nucleotide sequence encoding a protein of choice, the method comprising the steps of:
inserting nucleotide sequences encoding for the protein of choice into plasmid comprising at least one vir genes of Silicibacter sp. TM1040 strain of SEQ ID NO. 1;
infecting marine algae with the Silicibacter sp. strain and maintaining suitable condition for expressing the protein of choice by the marine algae; and
recovering the protein of choice.

A method for producing a heterologous peptide in an algae cell, the method comprising the steps of:
(a) transforming the algae cell with at least one nucleotide sequence selected from the group consisting of SEQ ID NO. 3, SEQ ID NO. 4, SEQ ID NO. 5, SEQ ID NO. 6, SEQ ID NO.7, SEQ ID NO. 8, SEQ ID NO. 9, SEQ ID NO. 10, SEQ ID NO. 11, SEQ ID NO. 12, SEQ ID NO. 13, and SEQ ID NO. 14 and a gene encoding the heterologous peptide;
(b) culturing the transformed algae cell under conditions that allow for expression of the gene encoding the heterologous peptide, thereby obtaining the peptide.

In a further aspect, the present invention provides for an antibacterial composition comprising isolated Silicibacter sp. TM1040, hereinafter TM 1040.

In another aspect, the present invention provides for an antibacterial composition comprising extracts and/or cell-free filtrates obtained from culture supernatants of TM1040 having a potent antibacterial activity that is effective in killing several pathogenic bacteria.

In yet another aspect, the present invention relates to an antibacterial composition comprising extracts and/or cell-free filtrates obtained from culture supernatants of TM1040 having a potent antibacterial activity that is effective in killing Mycobacterium marinum, Vibrio anguillarum, V. coralliilyticus, and V. shiloi bacteria.

A still further aspect of the present invention relates use of Silicibacter sp. TM1040, in a liquid culture, cell pellet (either wet or dehydrated), or the supernatant fluid from a culture of TM1040 as a probiotic composition that has applicability in coral reef maintenance or in commercial aquaculture of fin- and shellfish for prophylactic, preventative treatment or curative treatment of bacterial diseases or to improve growth and weight gain. The probiotic composition may be added to or cultured with greenwater in aquaculture feeds or mixed with algal feed prior to feeding. The bacteria could be provided alive, frozen, or dehydrated to be revived upon rehydration. Since TM1040 is a “natural” agent, it is compatible in the natural environment for treating fish diseases or preventing the bleaching of coral reefs.

Still a further aspect of the present invention relates to the use of Silicibacter sp. TM1040 as a producer of antibiotics against bacterial diseases of humans and animals, as a probiotic in commercial aquaculture and the aquarium fish hobby trade, and as an agent to maintain coral health and prevent coral bleaching, both in the coral reef hobby trade, as well as in the wild.

In yet another aspect, the present invention relates to Silicibacter sp. TM1040 that is grown using large-scale fermentation system for isolation and extracting of the culture medium isolates having antibacterial activity. Preferably, the Silicibacter sp. TM1040 is grown under shaking and aeration mode to increase production of antibacterial extracts.

Yet another aspect of the present invention relates to a method for producing antibacterial extracts from Silicibacter sp. TM 1040 by fermentation, the method comprising:
culturing the Silicibacter sp. TM1040 in a culture medium suitable for the growth of the bacterium and production of antibacterial extract; and
separating the extract from the culturing medium.

In yet another aspect, the present invention provides for a biofouling/biofilm inhibitor comprising a sufficient amount of TM1040 bacteria or extract therefrom to prevent or reduce the accumulation of other organisms on submerged marine surfaces including the hulls of ships, sonar domes, or any underwater surface. Preferably, TM1040 adheres to surfaces to establish a biofilm that produces antibacterial activity, preventing the attachment of other bacteria. Further, the purified compound(s) in the TM1040 supernatant may be added to paints or other materials applied to submerged surfaces.

In another aspect, the present invention relates to inclusion of genes responsible for the TM1040 antibacterial activity in an expression vector to be transfected into a compatible bacteria that is found in mammal thereby extending the antibiotic activity into humans or other animals. For example, common intestinal bacterium such as E. coli may be used so that these bacteria would become part of the animal's intestinal flora and produce the antibacterial activity. This could eliminate the need for adding antibiotics to the animal's feed as is currently practiced.

Other aspects and advantages of the invention will be more fully apparent from the ensuing disclosure and appended claims.

### BRIEF DESCRIPTION OF THE FIGURES

FIG. 1 shows the pathways involved in the catabolism of DMSP. Degradation of DMSP can occur by the lyase pathway, which involves the hydrolysis of the C-3 carbon of DMSP, producing acrylate and DMS (reaction 1), or by a series of demethylation steps (reactions 2 to 5). The first step in the demethylase pathway is demethylation of the DMS moiety of DMSP, producing MMPA (reaction 2). MMPA may be further demethylated to MPA (reaction 4), followed by the elimination of hydrogen sulfide (reaction 5), yielding acrylate, or in a demethiolation reaction (reaction 3), producing acrylate and MeSH. In some cases, the DMS produced by the lyase pathway may be oxidized to dimethyl sulfoxide (reaction 6).

FIG. 2 shows representative GC-FID chromatograms of DMS. DMSP in Pfiesteria dinoflagellates was detected as DMS, the major peak in each chromatogram (retention time, 2.6 min), after alkaline hydrolysis of DMSP. The numbers to the left of the DMS peak represent the peak area. The concentration of DMSP, as DMS, was determined by measurement of the peak area and comparison to a set of known standard concentrations of DMSP. The peaks shown were obtained from cultures containing 37,642 P. piscicida cells per ml and 50,985 P. shumwayae cells per ml. The minor peaks that display shorter retention times than DMS are not associated with DMSP, DMS, or other DMSP catabolites.

FIG. 3 shows the degradation of DMSP by Pfiesteria and Pfiesteria-like dinoflagellate cultures. DMSP was added to dinoflagellate cultures that contained both the dinoflagellates and their associated bacteria, and the loss of DMSP was measured over time using GC-FID. The results are presented for Cryptoperidiniopsis sp. strain CCMP1829 (•), P. piscicida CCMP1830 (▪), P. piscicida CCMP1921 (▴), P. piscicida CCMP1834 (▾), and P. shumwayae CCMP2089 (♦), and the negative control was medium alone (◯). The error bars represent the standard errors in three separate experiments with each culture.

FIG. 4 shows degradation of DMSP by mixed communities of culturable heterotrophic bacteria obtained from either Pfiesteria or Pfiesteria-like dinoflagellate cultures. Production of the DMSP catabolites, acrylate, DMS, MeSH, and MMPA was measured after 1 mM DMSP was added to the mixed bacterial suspensions. Shown are the mixed bacterial communities obtained from P. piscicida CCMP1830 (A), Cryptoperidiniopsis sp. strain CCMP1829 (B), and P. shumwayae CCMP2089 (C). The production of DMSP catabolites, MeSH (▪) and DMS (•), was measured using GC-FID (left axis), while the production of MMPA (▴) and acrylate (◯) was measured using high-performance liquid chromatography (right axis). These experiments were repeated three or more times with similar results.

FIG. 5 shows the degradation of DMSP by four pure-culture isolates of bacteria obtained from cultures of P. piscicida CCMP1830. (A) The data are presented as the percentages of 100 μM DMSP remaining after incubation with either bacterial strain TM1035 (▪), TM1038 (•), TM1040 (♦), or TM1042 (▴), or the control (◯), 100 μM DMSP without inoculum. (B) Comparison of the production of the DMSP catabolites (DC), MMPA, DMS, and MeSH, by the four bacterial strains after 3 h of incubation in the presence of 1 mM DMSP. The error bars represent the average standard error from three separate experiments.

FIG. 6 shows the kinetics of DMSP metabolism in four DMSP-degrading bacterial isolates from cultures of P. piscicida 1830. The symbols represent the amounts of DMSP (◯), MMPA (▴), MeSH (▪), and DMS (•) measured in the cultures of TM1035 (A), TM1042 (B), TM1038 (C), and TM1040 (D). The graphs represent an average data set whose mean was reproducible over several repeated experiments.

FIG. 7 shows the taxonomic analysis of the four DMSP-degrading bacteria isolated from P. piscicida. The phylogenetic tree was inferred from comparative sequence analysis, using 1,047 bp of 16S rDNA, generated by the neighbor-joining method and the Jukes-Cantor distance algorithm. The resulting tree shows the relationships among the Roseobacter clade bacteria associated with P. piscicida CCMP1830; strains TM1035, TM1038, TM1040, and TM1042; and nucleotide sequences of other known Roseobacter clade bacteria. Bootstrap values (n=1,000 replicate resamplings) are indicated for the neighbor-joining method where values are >500. An ‘f’ or ‘p’ indicates that the Fitch or parsimony method (respectively) is in agreement with the neighbor-joining tree. Bar=0.1 units of evolutionary distance.

FIG. 8 shows a TEM of Silicibacter sp. strain TM1040. A culture of TM1040 was prepared in half-strength 2216 marine broth and grown for 20 h at 30° C. without agitation. Motile cells were blotted onto copper disks, stained with 1% uranyl acetate, and visualized by TEM. Three simple lophotrichous flagella attached to the pole of the cell are apparent (arrow). Bar=0.5 μm.

FIG. 9 shows the effects of starvation on the chemotactic response of Silicibacter sp. strain TM1040. The chemotaxis of unstarved versus starved cells to 200 μM methionine was measured using the capillary assay (Materials and Methods). Chemotaxis of starved cells to either methionine (black bars) or buffer lacking nutrient (white bars) is compared to the response of unstarved cells either to methionine (dense cross-hatched bars) or to the buffer lacking nutrient (sparse cross-hatched bars). The response factor, calculated by dividing the mean number of bacteria in methionine-filled capillaries by the mean number of bacteria in buffer-filled capillaries, is significantly increased in the starved cells (▪) compared to that in the unstarved cells (◯). The values shown are the mean and standard deviation (n=12).

FIG. 10 shows the chemotaxis of Silicibacter sp. strain TM1040 toward cell homogenates. Chemotaxis of TM1040 toward heated (80° C.) (white bars) and untreated (black bars) homogenates was measured using the capillary chemotaxis assay (Materials and Methods). The response factor is calculated by dividing the mean number of bacteria in homogenate-filled capillaries by the mean number of bacteria in buffer-filled capillaries. The chemotactic response of TM1040 to the untreated dinoflagellate homogenate is >2.5 times greater than the response to either algal or bacterial homogenates, and heating the dinoflagellate homogenate reduces the response factor by ca. 40%. The values shown are the mean and standard deviation (n=8).

FIG. 11 shows screening of putative attractant compounds using the qualitative chemotaxis assay. Chemotaxis behavior of TM 1040 was assessed using BM glycerol motility agar on which a compound to be tested is placed (indicated by the dot to the left of the swimming colony). (A) Chemotaxis of Silicibacter sp. strain TM1040 through BM glycerol motility agar results in an ever-increasing colony of motile bacteria that is punctuated by one or more internal bands of bacteria (indicated by the arrows) and the periphery (diamondhead arrow). Shown is the response of TM1040 to DMSP (B), methionine (C), valine (D), arabinose (E), and fructose (F). In the presence of either DMSP, methionine, or valine, the outer ring is disturbed and becomes asymmetrical, indicating a chemotactic response from the cells. In contrast, neither arabinose nor fructose causes a change in the symmetry of the colony, suggesting that TM1040 does not respond to these compounds.

FIG. 12 shows quantitative measurement of the chemotactic response of Silicibacter sp. strain TM1040 to pure compounds. Chemotaxis of TM1040 was assessed using the capillary assay with a subset of potential attractants discovered using the qualitative assay. Capillaries were filled with either buffer (as a control) or 2 μM (white bars) or 200 μM (black bars) attractant. The response factor was calculated by dividing the mean number of bacteria in attractant-filled capillaries by the mean number of bacteria in buffer-filled capillaries. DMSP and amino acids produced the strongest chemotactic response from TM1040. NAG, N-acetylglucosamine; Pro, propionate; AKG, α-ketoglutarate. The values shown are the means and standard deviations (n=12).

FIG. 13 is a cladogram showing the homology of Silicibacter sp. TM1040 Vir protein homologs with known Vir and Tra proteins from other bacterial species. GenBank accession numbers are used for the homologs.

FIG. 14 shows the genetic map of the Silicibacter sp. TM1040 vir gene loci. The first locus contains homologs to virD2 and virD4, required for producing the coupling protein and energetics of DNA transfer. The second locus, physically separated from the first locus by ca. 16 kb, includes homologs encoding VirB1-B6 and VirB8-B11. The VirB proteins function in the synthesis of the channel, chaperones, and pilus structures required for cell-cell contact transfer of the DNA.

FIG. 15 shows the visualization and co-localization of Silicibacter sp. TM1040 cells interacting with P. piscicida. Bacteria were pre-stain with a fluorescent tracer dye and added to washed P. piscicida zoospores. After two hours, samples were removed, chemically fixed, and viewed by phase contrast (A) and fluorescence microscopy (B). (C) The phase-contrast and fluorescent images of the same specimen were overlaid, and the bacteria co-localized with the dinoflagellate cells as described in Materials and Methods. Numerous clusters of fluorescent bacteria can be seen colocalized to a crescent-shaped area within the periphery of a settled zoospore. The bar represents 10 um.

FIG. 16 shows serial Z-section composite image of the interaction between Silicibacter sp. TM1040 and P. piscicida. The bacteria were fluorescently labeled as described in Materials and Methods, and the samples were chemically fixed and visualized using confocal microscopy. Optical Z-section slices through individual dinoflagellates were captured in 0.5 μm increments to create a series of images through the Z-axis of the cell (proximal to distal surface of the zoospore). The resulting series was then color coded according to depth (Z-axis length) and merged into a single image, where objects tinted in green are nearer the viewer, those in shades of red and pink are in the midsection of the dinoflagellate cell, and objects near the distal surface are represented in shades of blue. Many of the bacterial cells are observed to co-localize to the middle depths of the image, suggestive of being in or near the cytoplasm of the dinoflagellate. The bar represents 10 μm.

FIG. 17 shows the motility screening of transposon-insertion mutants. Mutant strains obtained from random transposon insertional mutagenesis were screened for their ability to swim through semi-solid Mot agar. This assay identifies mutations in genes encoding structural components of the flagellum, hook basal body, and motor, chemotaxis signal transduction proteins, as well as global regulators of flagellar gene transcription. Clockwise from upper left are Silicibacter sp. TM1040 (wild-type motility), plus three strains with motility defects (TM2014, TM2017, and TM2038). Strains TM2014 and TM2017 are non-motile by this assay, while TM2038 produces flares of motile cells.

FIG. 18 shows that non-motile mutants lack flagella. Flagellar filaments were observed by phase-contrast microscopy after staining with silver nitrate according to the method of West et al. (1977). (A) Silicibacter sp. TM1040 (wild type); (B) TM2014; (C) TM2017, and (D) TM2038. The arrows point to flagellar filaments observed in both the wild-type and TM2038 cells. The bar represents 1 μm.

FIG. 19 shows the mutation in strain TM1038 leads to elongated cells. The cellular morphology of Silicibacter sp. TM1040 and the three Mot-mutant strains was observed by phase-contrast microscopy after incubation of the cells to the mid-exponential phase of growth. The cells of the parent (strain TM1040; A), TM2014 (B), and TM2017 (C) possess wild-type cell morphology, with a mean cell length of ca. 1.6 μm, while cells of strain TM2038 (D) are elongated (mean of 6.9 μm). The bar represents 5 μm.

FIG. 20 shows the genomic analysis of the mutations leading to Mot− phenotype. Mot-strain TM2014 has a transposon insertion in a gene of unknown function (henceforth referred to as flaA) that resides in a group of genes with the same transcriptional orientation, and that appear to encode proteins required for flagellar export and/or rotation. The mutation in TM2017 affects a gene that encodes a protein with strong homology to CckA, while the mutation in TM2038 lies in a gene whose deduced amino acid sequence has homology to CtrA. In other genera, CckA, a histidine kinase, and CtrA, a DNA-binding response regulator, compose a two-component regulatory circuit that regulates flagellar synthesis and other metabolic functions.

FIG. 21 shows the serial Z-section composite images of the interaction between Mot− mutants of Silicibacter sp. TM1040 and P. piscicida. The labeling of the bacteria and preparation of the composite Z-section images for confocal microscopy is as described in Materials and Methods. The depth of an object is color-coded, such that green objects are nearer the viewer (on the proximal facing surface), reds and pinks are in the midsection of the zoospore, and blue objects are distal. The panels show composite Z-section images of (A) Silicibacter sp. TM1040, (B) TM2014 (flaA), (C) TM2017 (cckA), and TM2038 (ctrA) associated with a dinoflagellate zoospore. The two arrows in panel A denote TM1040 cells at the same depth as a food vacuole. All three mutations reduce the number of bacteria found to co-localize with the midsection of the zoospore.

FIG. 22 shows the quantitative measurement of the effect of Mot− mutations on bacterial attachment to the dinoflagellate cell. Wild-type and Mot− strains of Silicibacter sp. TM1040 were fluorescently labeled and incubated with washed zoospores. Following fixation, the samples were examined by confocal microscopy, and the depth of each bacterial cell determined relative to the proximal and distal surfaces of the dinoflagellate. The mean number of bacteria on the surface (black bars) or co-localized to the interior of the dinoflagellate (white bars) for each strain was determined as a percentage relative to wild-type cells. (A) The percent of wild-type co-localization to either the surface or interior of the dinoflagellate for TM1040 (wild type) and each Mot− mutant (TM2014, TM2017, and TM2038). (B) The percent of dinoflagellates in the population observed to possess bacteria on their surface or co-localized to their interior. Heat-killed Silicibacter sp. TM1040 cells served as a negative control in these experiments. Asterisks indicate that the mean of that sample is significantly different from the mean of the wild-type cells (P<0.05; n=180).

FIG. 23 shows the growth of axenic P. piscicida zoospores in the presence and absence of Silicibacter sp. TM1040 and three Mot− mutant strains. Add-back experiments were performed to analyze the contribution of wild-type and Mot− cells on the growth of P. piscicida. The panels show (A) P. piscicida and (B) Rhodomonas sp. prey algal cell density over the period of the experiment (9 days). The symbols represent: () Silicibacter sp. TM1040; (◯) no bacteria control; (□) heatkilled TM1040 cells; (⋄) TM2014 (flaA); (♦) TM2017 (cckA); and, (∇) TM2038 (ctrA).

FIG. 24 shows the analysis of the bacterial community in P. piscicida add-back experiments. DGGE of the PCR products amplified using an oligonucleotide primer set specific for eubacterial 16S rDNA was performed to ensure only Silicibacter sp. TM1040 or one of the three Mot-mutants populated the axenic P. piscicida cultures. This PCR results in a single, rapidly migrating DNA product from Silicibacter sp. TM1040 genomic DNA (lower band). A plastid DNA band amplified from the Rhodomonas sp. prey algal, added to all cultures, serves as an internal positive control (upper DNA band) and indicates the presence of the prey algal.

FIG. 25 shows testing results of extracts of TM1040 for their ability to inhibit the growth of V. anguillarum.

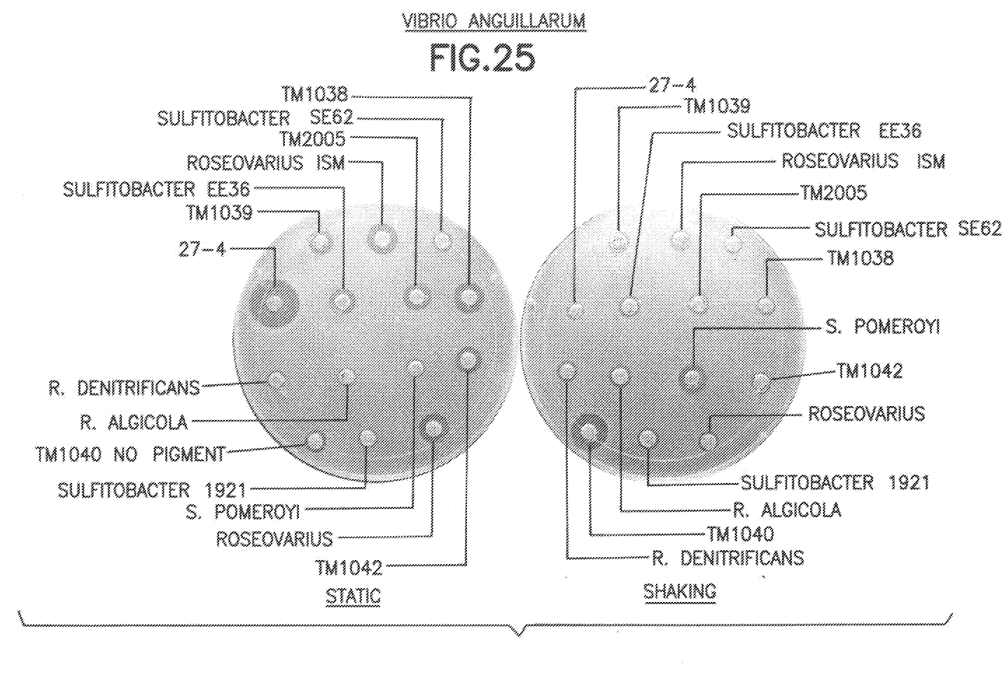

FIG. 26 shows that TM1040 grown with vigorous shaking produced a much larger zone of inhibition than the in a static mode.

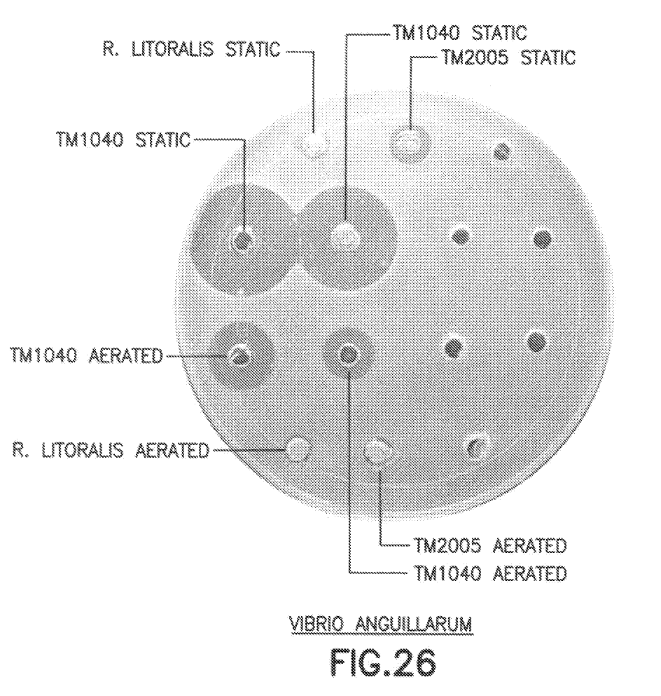

FIG. 27 shows testing results of extracts of TM1040 for their ability to inhibit the growth of M. marinum.

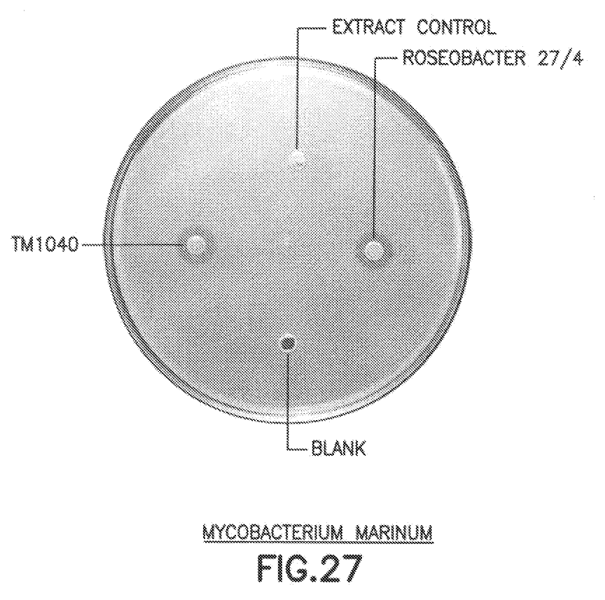

FIG. 28 shows testing results of extracts of TM1040 for their ability to inhibit the growth of V. coralliilyticus.

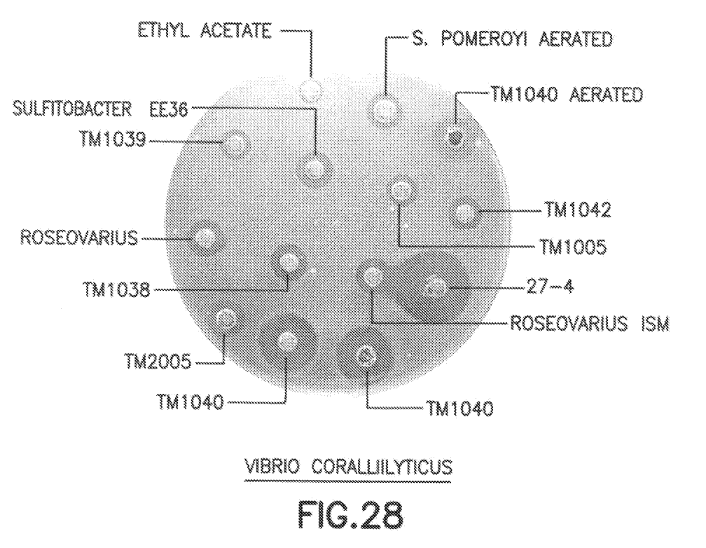

FIG. 29 shows testing results of extracts of TM1040 for their ability to inhibit the growth of V. shiloi.

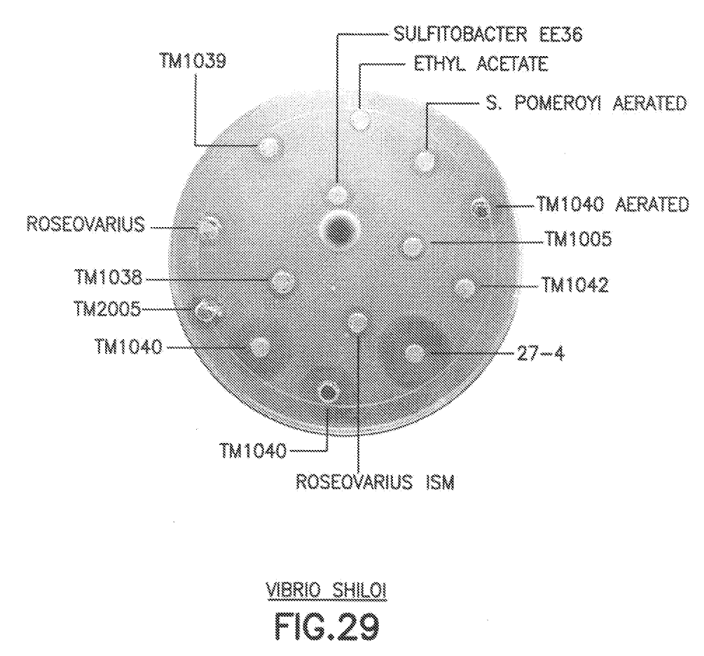

### DETAILED DESCRIPTION OF THE INVENTION

In the marine environment, dinoflagellates coexist and interact with a diverse community of bacteria and other microorganisms. These interactions can be studied in monocultures of dinoflagellates obtained from environmental samples. Within these cultures, bacteria native to the algal niche assimilate dinoflagellate-derived nutrients and are intrinsically propagated with the dinoflagellates in continuous subcultures. The bacterial community inhabiting several Pfiesteria dinoflagellate cultures isolated from the Chesapeake Bay, Md. was characterized. All of the dinoflagellate cultures examined contained one or more Roseobacter spp. representing the second most abundant clone obtained from 16S ribosomal DNA (rDNA) clone libraries. In addition, several bacteria were found attached to these dinoflagellates by using fluorescent in situ hybridization and confocal scanning laser microscopy. After a stringent washing procedure to remove unattached bacteria, the predominant bacterial species present was a bacterium closely related to Sulfitobacter pontiacus, a Roseobacter clade organism. Also, a Roseobacter sp. was found to be necessary for the growth of Pfiesteria in culture.

The present invention provides methods and compositions to create novel plasmids and vectors for transfecting algae. The present invention further provides novel methods and compositions for the generation of novel plasmids in stably transformed algae cells. Additionally, the novel plant plasmids produced in accordance with the present invention provide a valuable means for replicating and/or expressing a heterologous DNA sequence or gene in plants or plant cells, preferably algae cells. These heterologous DNA sequences or genes are typically associated with the production of proteins that make a plant more useful (e.g. proteins associated with or conferring herbicide resistance, disease resistance and/or crop yield), useful proteins to be recovered from plants or plant cells and proteins that cause the synthesis of chemicals or compounds that make a plant or plant cells more useful agriculturally or medicinally.

To date, several methodologies exist which provide for the delivery of DNA sequences into plants. These delivery systems include whole virus infection, the Agrobacterium Ti plasmid systems, free DNA application to abraded or cut plant surfaces, and free DNA introduction into plant protoplasts by means of, for example, polyethylene glycol, calcium phosphate, poly-L-ornithine, micro-injection, or electroporation. It is understood by those skilled in the art that the plasmid systems of the present invention will provide another system for delivering to plants, and preferably algae.

### Example 1

The production of DMSP by P. piscicida and P. shumwayae was measured and DMSP catabolism by the Pfiesteria cultures was assessed and the bacterial communities associated with them. New dinoflagellate-associated roseobacters capable of DMSP degradation by both the lyase and demethylation pathways were isolated and identified. The results show that both P. piscicida and P. shumwayae contain significant levels of DMSP. The DMSP contents of the two Pfiesteria species (3.44 to 4.25 μM) (Table 1) are similar to those measured in other dinoflagellates.

TABLE 1
Intracellular DMSP contents of Pfiesteria species

| | | Intracellular DMSP | | |
|---|---|---|---|---|
| Species | Strain | (pg per cell) | Cell vol (nl) | DMSP (μM) |
| P. piscicida | 1830 | 0.41 | 0.69 ± 0.12 | 3.44 ± 1.00 |
| P. shumwayae | 2089 | 0.40 | 0.55 ± 0.02 | 4.25 ± 1.47 |

For example, the intracellular DMSP concentrations in photosynthetic species, such as Prorocentrum, Gymnodinium, and Amphidinium species, are reported to be 1 to 10 μM (Keller et al., 1996). Interestingly, the concentration of DMSP in another heterotrophic dinoflagellate, Crypthecodinium cohnii, has been reported to be 10 μg per cell (Keller et al, 1996), a value that is much higher than those observed in either Pfiesteria species (about 0.4 μg per cell). This difference undoubtedly reflects physiological and taxonomic differences between Pfiesteria and Crypthecodinium and underscores the difficulty in making general statements about DMSP physiology among taxonomically diverse dinoflagellate species.

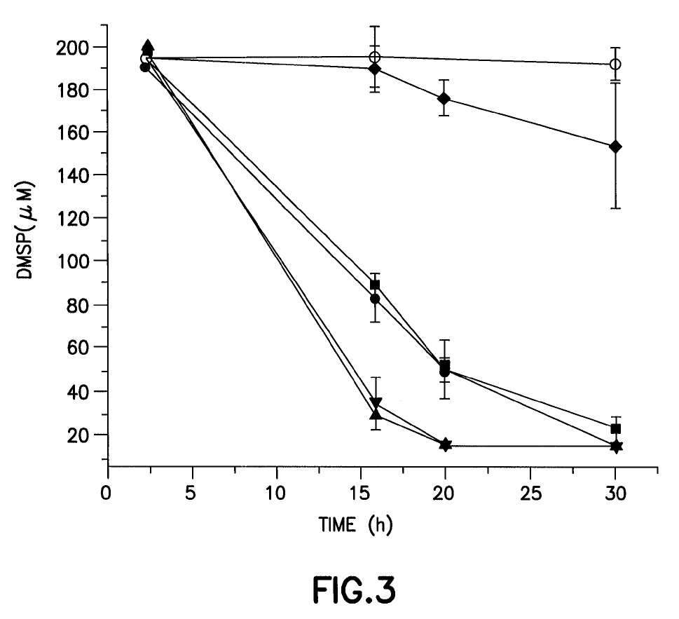

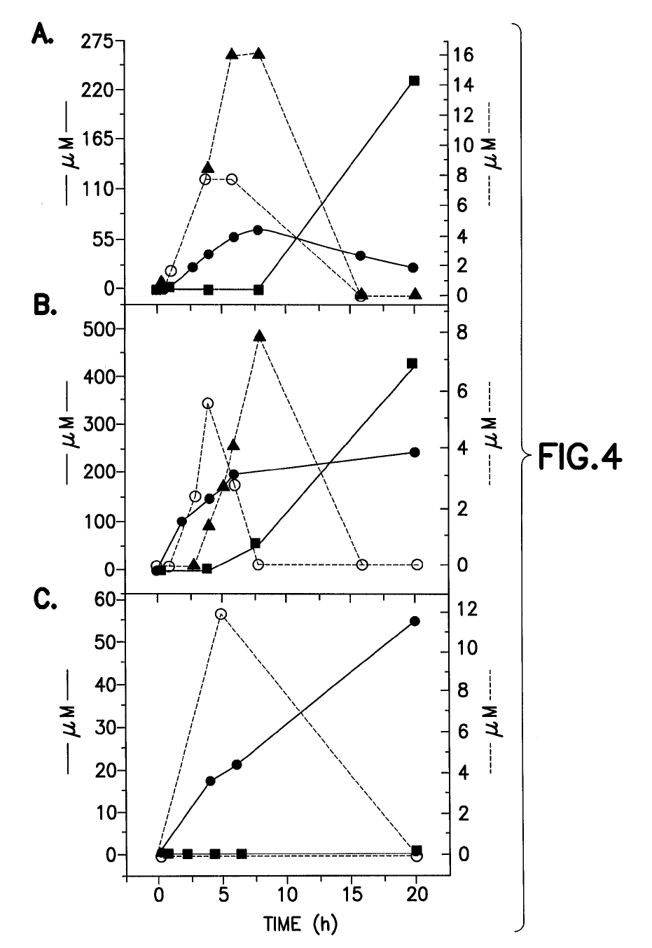

Both P. piscicida and P. shumwayae contain significant levels of DMSP, yet the P. piscicida cultures that include dinoflagellates plus associated bacteria degrade DMSP at significantly higher rates as shown in FIG. 3. Thus, it is conceivable that differences in the compositions of the bacterial communities may affect the rates of DMSP decomposition in P. piscicida and P. shumwayae cultures. The data in FIG. 4 showing the difference between DMSP catabolite kinetics in the mixed culturable heterotrophic bacteria support this idea. Other factors may also be important, including the possibility that slow-growing bacteria in the P. shumwayae culture may have a low rate of DMSP degradation or that the concentration of DMSP used may have a deleterious effect specific to the P. shumwayae bacterial community.

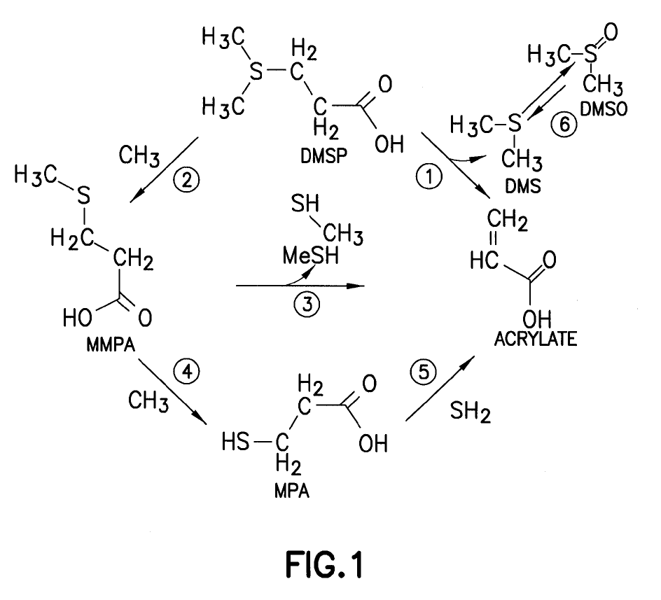

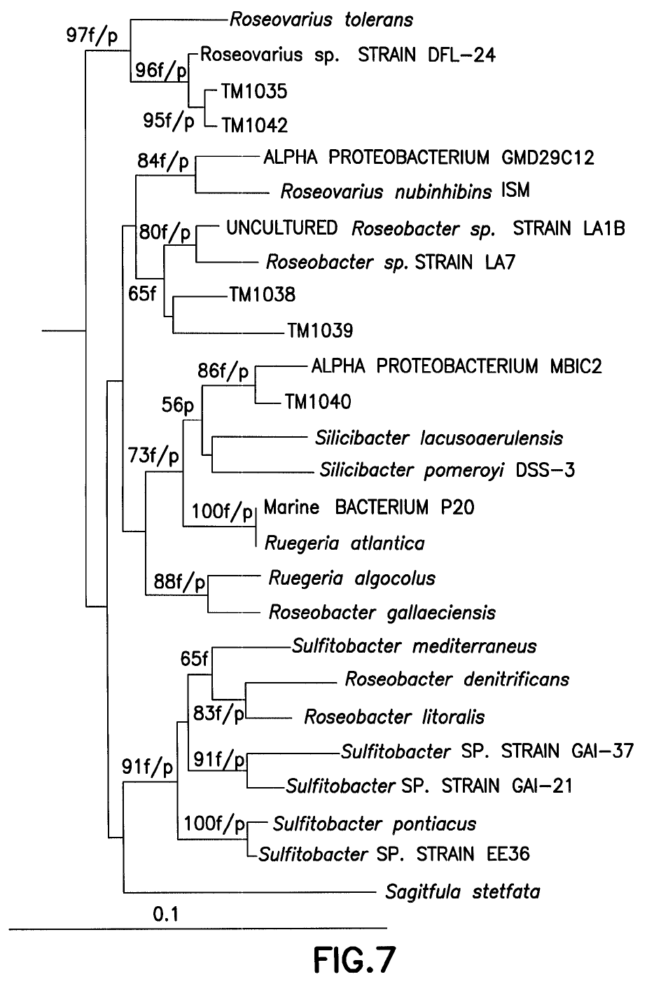

Four DMSP-degrading bacterial isolates were obtained from P. piscicida cultures and were found to be phylogenetically related to members of the Roseobacter clade as shown in FIG. 7. As shown in the taxonomic tree, two of the isolates (TM1035 and TM1042) are most closely related to Roseovarius species, while TM1038 and TM1040 are unique Roseobacter species not related to known roseobacters. Despite their taxonomic differences, all four bacteria shared the common trait of demethylating DMSP to MMPA, while strains TM1035 and TM1042 further metabolize DMSP to produce DMS, indicating that demethylation is a major pathway by which Pfiesteria-associated roseobacters degrade DMSP. Equally interesting are the DMSP demethylation pathways used by these strains. As an example, TM1038 demethylates DMSP to produce MMPA and MeSH, a pathway that appears to be commonly used by marine bacteria (Kiene et al. 1996). In contrast, TM1040 strictly demethylates DMSP to produce MMPA without MeSH, a pathway reported to be used by one other aerobic marine bacterium, strain BIS-6, isolated from Biscayne Bay, Fla. (Visscher et al., 1994). This bacterium demethylates DMSP to MMPA as shown in FIG. 1, reaction 2, followed by a further demethylation to MPA, reaction 4. Although not measured in this study, TM1040 is likely also to produce MPA instead of MeSH, as has been observed for BIS-6.

The two Roseovarius-related strains, TM1035 and TM1042, are capable of both demethylation and lyase cleavage of DMSP. The presence of dual demethylation-lyase pathways in the same organism is a recently discovered phenomenon. Gonzalez et al. (1999) reported that 5 out of 15 DMSP-catabolizing bacteria isolated from Georgia coastal seawater and the Caribbean Sea catabolized DMSP to produce both DMS and MeSH, as well as converted MMPA to MeSH. One of these five isolates was taxonomically identified as R. nubinhibens ISM. The capacity to use both DMSP pathways may provide these bacteria with a survival advantage, especially in environments where DMSP concentrations are high, such as the phycosphere surrounding DMSP-producing dinoflagellates. Bacteria that utilize the lyase cleavage pathway are capable of growing on DMSP as a sole carbon source (Yoch et al., 2002). In contrast, while the demethylation pathway does not always lead to increased growth (Jansen et al., 2001), much of the sulfur obtained from this pathway is utilized for protein synthesis and seems to be preferred over other sources of sulfur abundant in seawater (Kiene et al, 1999). Thus, coupling of both DMSP-degradative pathways in the same organism may satisfy both the carbon and sulfur requirements of these dinoflagellate-associated marine bacteria.

In analyses of DMSP catabolism, it was occasionally observed that the sum of the DMSP catabolites produced did not always equal the amount of DMSP lost from the culture. There are several possible explanations for this. First, bacterial enzymes may have degraded the DMSP catabolites shortly after they were produced. This is a strong possibility in light of the high reaction rates observed. A good example supporting this is the failure to detect acrylate production in either TM1035 or TM1042, despite the presence of detectable levels of DMS, which constitutes the other half of lyase cleavage of DMSP. The absence of acrylate is most likely due to rapid conversion, a finding that was also noted by Ansede et al. (Ansede et al., 1999), who, using nuclear magnetic resonance analysis, were unable to detect acrylate production by a Roseobacter species even though DMS was produced. The present results also agree with environmental studies that show rapid degradation of acrylate by bacterial communities associated with algal cells or debris (Osing a et al., 1997).

### Material and Methods

### Dinoflagellate Strains and Culturing.

Dinoflagellate cultures of P. piscicida CCMP1830, CCMP1921, and CCMP1834; Cryptoperidiniopsis sp. strain CCMP1829; and P. shumwayae CCMP2089 (Provasoli-Guillard National Center for Culture of Marine Phytoplankton) were grown as previously described (Alavi et al 2001). The dinoflagellates were fed the prey alga Rhodomonas sp. strain CCMP768 continuously as needed. Rhodomonas sp. was grown in 35 practical salinity units (psu) F/2 medium lacking silica at 20° C. under a 14-h light, 10-h dark cycle (Guillard et al., 1975). For all assays, the dinoflagellates were grown to a maximum cell density of approximately 10⁵ per ml, whereupon feeding was stopped for 36 h, which allowed complete removal of the Rhodomonas algae (monitored by inverted microscopy).

### Bacterial Strains and Media.

Bacteria were isolated from P. piscicida CCMP1830 culture by first spreading a 10-fold dilution series of the dinoflagellate culture on 0.5× Zobell marine agar 2216 (18.7 g of Difco marine broth 2216, 15 g of Difco Bacto Agar, and 1,000 ml of distilled H₂O), hereafter referred to as marine agar. After 5 to 7 days of incubation at 30° C., colonies with unique morphologies were picked at random and streaked to purity on marine agar, resulting in the strains TM1034 to TM1042. The bacterial cultures were used after incubation in marine broth (same as marine agar but lacking the agar) at 30° C. in a shaking water bath for 1 to 3 days. Escherichia coli INVαF′ was grown in Luria-Bertani (LB) broth (Ausubel, F. M. 2001) or on LB agar containing 1.5% Bacto Agar (Becton Dickinson, Franklin Lakes, N.J.).

### Chemicals

DMSP was synthesized from acrylate and DMS according to the method of Chambers et al., 1987. The purity of the resulting DMSP was confirmed by chemical analyses, including flash point, melting point, and total C, H, O, N, S, and Cl (Galbraith Laboratories, Inc., Knoxville, Term.). MMPA was synthesized by alkaline hydrolysis of its methyl ester, methyl-3-(methylthio)propionate (Aldrich, Milwaukee, Wis.) (Jansen et al., 1998). Other organic sulfur compounds were purchased from Aldrich. All chemicals used were of the highest purity commercially available.

### DMSP Content and Metabolism.

To measure the DMSP contents of the dinoflagellates P. piscicida CCMP1830 and P. shumwayae CCMP2089 and the prey alga Rhodomonas sp. strain CCMP768, cells were grown to a maximum density of approximately 10⁵ per ml in 500-ml batch cultures, except for Rhodomonas sp., which was grown in 1-liter batch cultures to a maximum density of 10⁶ cells per ml. The abundance and cellular volume of the cells were measured in the 7- to 20-μm-diameter particle range from three or more 1-ml samples of culture using a Coulter Multisizer II particle counter (Becton-Dickinson). Because the particle counter does not distinguish between Pfiesteria dinoflagellates and Rhodomonas prey algae, all dinoflagellate cultures were starved to reduce the Rhodomonas population to below detectable limits.

DMSP was measured in 2-ml whole-culture aliquots and in concentrated cell lysates. To obtain concentrated cell lysates, 200-ml culture aliquots were centrifuged at 4,000×g for 15 min and the cell pellets were resuspended in 2 ml of sterile distilled water on ice. For Rhodomonas sp. strain CCMP768, multiple cell pellets were combined and resuspended in a final 2-ml aliquot of sterile distilled water. A cell homogenate of each sample was then obtained using a Sonic Dismembrator sonicator (Fisher, Hampton, N.H.). DMSP was measured in 2-ml samples as described in “Analytical techniques” below.

To measure the degradation of DMSP by dinoflagellate and prey algal cultures, cells were grown as described above and the cell density was normalized across all cultures to 10⁴ per ml by diluting the cultures with sterile medium. DMSP was added to the culture from a sterile neutralized stock at a final concentration of 200 μM, and its degradation was measured at intervals throughout the duration of the experiment, as described in “Analytical techniques” below.

The catabolism of DMSP by the bacterial component of each culture was measured in suspensions containing a mixture of dinoflagellate-associated bacteria that were isolated as follows. A 10-fold dilution series of each dinoflagellate culture at peak dinoflagellate density (approximately 10⁵ cells per ml) was spread on marine agar and incubated at 30° C. for 5 days. The resulting colonies from plates containing 50 to 200 colonies were resuspended from the agar surface using sterile 10-psu artificial seawater (Instant Ocean, Mentor, Ohio) and washed twice by centrifugation at 14,000×g, whereupon the optical density was normalized to 0.6 at 600 nm for each suspension. An aliquot of DMSP was then added from a sterile neutralized stock to a final concentration of 1 mM, and DMS, MeSH, MMPA, and acrylate were measured as described in “Analytical techniques” below.

DMSP catabolism was also measured in four bacterial strains (TM1035, TM1038, TM1040, and TM1042) isolated from the dinoflagellate culture. Each strain was grown in a 50-ml marine broth culture amended with 1 mM DMSP to induce the production of enzymes necessary for DMSP catabolism. The cultures were grown to an optical density at 600 nm of 0.6 and washed twice with sterile 10-psu artificial seawater. DMSP was added to a final concentration of either 0.1 or 1 mM, and 1-ml aliquots of the bacterial cultures were dispersed into 26-ml serum bottles. The bottles were immediately capped with a butyl rubber septum and incubated at 30° C. with shaking. At intervals throughout the experiment, samples were sacrificed for measurement of DMSP, DMS, MeSH, acrylate, and MMPA as described below.

### Analytical Techniques.

DMSP was measured as DMS following alkaline hydrolysis. An aliquot of the sample was added to a 26-ml serum bottle with the addition of an equal volume of either 5 M NaOH or distilled water, and the bottle was capped with a butyl rubber septum. Solutions of pure DMSP at 1 to 500 μM dissolved in distilled water were prepared in exactly the same manner in parallel with each experiment. After overnight incubation, DMS resulting from alkaline hydrolysis of DMSP was measured in 500 μl of headspace gas using a Hewlett-Packard 5890 gas chromatograph equipped with flame ionization detection (GC-FID). DMS produced without alkaline hydrolysis was subtracted from the total, and the result was compared to DMS produced from the hydrolysis of pure DMSP standards to obtain the final molar concentration of DMSP in the unknown sample. The retention time of DMS was determined by injecting 50 μl of headspace gas from a capped serumbottle containing 5 μl of pure DMS that had completely volatilized.

DMS and MeSH production in the cultures was measured by direct sampling of 500 μl of headspace gas without prior alkaline hydrolysis of the sample. The concentration of DMS was determined using standard curves generated from known concentrations of DMS produced by complete alkaline hydrolysis of known amounts of DMSP. The concentrations of gaseous MeSH in the cultures were determined from standard curves using a dilution series of pure MeSH gas.

The presence and concentrations of acrylate and MMPA in the cultures were measured by high-performance liquid chromatography (Agilent [Palo Alto, Calif.] 1100 equipped with diode array detection) and a Zorbax XDB C₁₈ column (2.1 by 150 mm; 5-μm pore size) (Agilent) according to the method of Ansede et al. 1999.

### Statistics.

The Mann-Whitney test for two independent samples was used to compare the DMSP contents of P. piscicida CCMP1830 and P. shumwayae CCMP2089. The apparent first-order rate constants for DMSP degradation and catabolite production by bacterial strains TM1035, TM1038, TM1040, and TM1042 was calculated using a linear least-squares regression analysis of the data, where the slope of the line equals the first-order rate constant (r>0.90).

### DNA Methods.

Chromosomal DNA was extracted from bacterial cells by routine methods (Sambrook et al., 1989) and used as a template in a PCR to amplify the near-full-length (approximately 1,300-bp) 16S rDNA gene. The PCR conditions were as previously described (Alavi et al., 2001). The resulting PCR products were analyzed by electrophoresis using a 1.0% agarose gel in 1×TAE (Ausubel, 2001) to confirm the presence of a single 1,300-bp product, which was then excised from the gel using a sterile razor blade, purified using the QIAGEN gel extraction kit, and cloned into the TA cloning vector pCR2.1 (Invitrogen, Carlsbad, Calif.) under the ligation conditions recommended by the manufacturer. Plasmid DNA was transformed into E. coli INVαF′ competent cells (Invitrogen). Transformants were selected and screened for DNA insertion using LB agar containing kanamycin (80 μg per ml) plus X-Gal (5-bromo-4-chloro-3-indolyl-β-D-galactopyranoside; 40 μg per ml). White colonies, i.e., those harboring recombinant plasmids, were picked at random and grown overnight with antibiotic selection. Plasmid DNA was extracted by alkaline lysis and purified by standard methods using a cesium chloride gradient. The presence of a near-full-length 16S rDNA insert was confirmed by agarose gel electrophoresis analysis of EcoRI-digested plasmid DNA. The nucleotide sequence of each 16S rDNA was determined as previously described (Alavi et al., 2001).

### Nucleotide Sequence Analysis and Phylogenetic-Tree Construction.

The construction of phylogenetic trees was done as described by Alavi et al. 2001.

### Nucleotide Sequence Accession Numbers.

The GenBank accession numbers for the 16S rDNA sequences used to generate phylogenetic trees are as follows: Roseovarius tolerans, Y11551; Roseovarius sp. strain DFL-24, AJ534215; marine bacterium ATAM407-61, AF359525; Sagittula stellata, U58356; α-proteobacterium GMD29C12, AY162070; Roseovarius nubinhibens, AF098495; uncultured Rhodobacter LA1-B32N, AF513928; Roseobacter sp. strain LA7, AF513438; marine bacterium HP29w, AY239008; α-proteobacterium MBIC1887, AB026492; Silicibacter lacuscaerulensis, U77644; Silicibacter pomeroyi, AF098491; marine bacterium P20, AY082668; Reugeria atlantica, AF124521; Reugeria algocolus, X78313; Roseobacter gallaciensis, Y13244; Sulfitobacter mediterraneus, Y17387; Roseobacter denitrificans, X69159; Roseobacter litoralis, X78312; Sulfitobacter sp. strain GAI-37, AF007260; Sulfitobacter sp. strain GAI-21, AF007257; Sulfitobacter pontiacus, Y13155; and Sulfitobacter sp. strain EE36, AF007254. The nucleotide sequences incorporating 1,300 bp of the 16S rDNA gene from strains TM1035, TM1038, TM1040, and TM1042 have been deposited in the GenBank database under accession numbers AY332660, AY332661, AY332662, and AY332663, respectively.

### Results

DMSP content of P. piscicida and P. shumwayae.

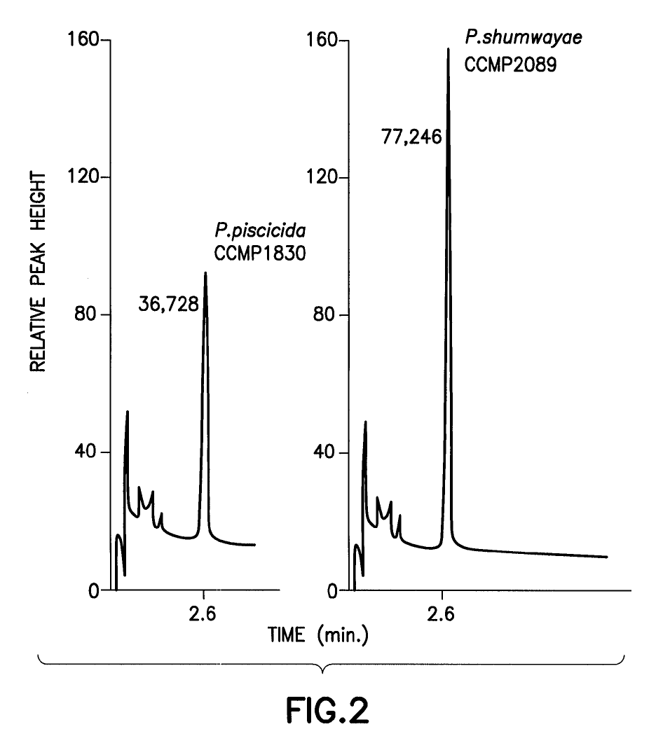

FIG. 2 shows representative chromatograms of DMSP in cell lysates from each Pfiesteria species. The internal cell volumes for P. piscicida and P. shumwayae were determined to be 0.69 and 0.55 nl, respectively; thus, the average intracellular DMSP concentration of P. piscicida is 3.44 μM, while that of P. shumwayae is estimated to be 4.25 μM as shown above in Table 1. Statistical analyses show that the mean DMSP concentrations and the mean intracellular volumes for the species are not significantly different (P>0.05).

Intracellular DMSP was not detected in the Rhodomonas prey algal cultures, even when 1.000-fold more cells were used for the analysis. Thus, Rhodomonas sp. strain CCMP768 is not a significant source of DMSP in the Pfiesteria culture. While DMSP was readily detectable in Pfiesteria cell lysates, it was not found in either supernatants or whole-cell samples.

### Degradation of DMSP by Dinoflagellate Cultures.

Cultures of P. piscicida, P. shumwayae, and a taxonomically similar dinoflagellate, Cryptoperidiniopsis sp., lacking Rhodomonas, as well as a culture of the prey algae, were analyzed for the ability to degrade DMSP. While Rhodomonas cultures failed to degrade DMSP (data not shown), P. piscicida and Cryptoperidiniopsis cultures degraded exogenously added DMSP within 20 to 30 h of incubation as shown in FIG. 3. In contrast, the P. shumwayae culture was much slower to degrade DMSP, requiring >72 h to achieve complete degradation of the DMSP (data not shown). P. piscicida CCMP1830, Cryptoperidiniopsis sp. strain CCMP1829, and P. shumwayae CCMP2089 were chosen for further analysis.

### DMSP Catabolism by the Dinoflagellate-Associated Bacterial Consortium.

The contribution of the bacterial community in the dinoflagellate cultures to degrading DMSP was assessed. Three separate mixed communities of culturable heterotrophic bacteria from cultures of P. piscicida CCMP1830, Cryptoperidiniopsis sp. strain CCMP1829, and P. shumwayae CCMP2089 were isolated, and their abilities to degrade DMSP were measured. The bacterial suspensions from the P. piscicida and Cryptoperidiniopsis cultures catabolized DMSP, initially producing DMS and acrylate, followed by the production of MMPA and MeSH as shown in FIGS. 4A and B. The concentrations of MeSH and DMS were consistently much higher (˜100-fold) than the concentration of either MMPA or acrylate. In general, the concentrations of the DMSP catabolites eventually decreased over 20 h, except for MeSH gas, which continued to increase throughout the period. In contrast to these results, the bacterial suspension obtained from the P. shumwayae culture produced only DMS and acrylate and failed to produce either MMPA or MeSH. These data demonstrate that one or more species of DMSP-degrading bacteria are associated with the P. piscicida and Cryptoperidiniopsis dinoflagellates and that both the demethylase and lyase pathways are active. Since the bacterial communities from both the P. piscicida CCMP1830 and Cryptoperidiniopsis sp. strain CCMP1829 cultures showed similar profiles of DMSP catabolism, only bacteria from the P. piscicida CCMP1830 culture were selected for further analysis.

### DMSP Catabolism by Isolated Dinoflagellate-Associated Bacteria.

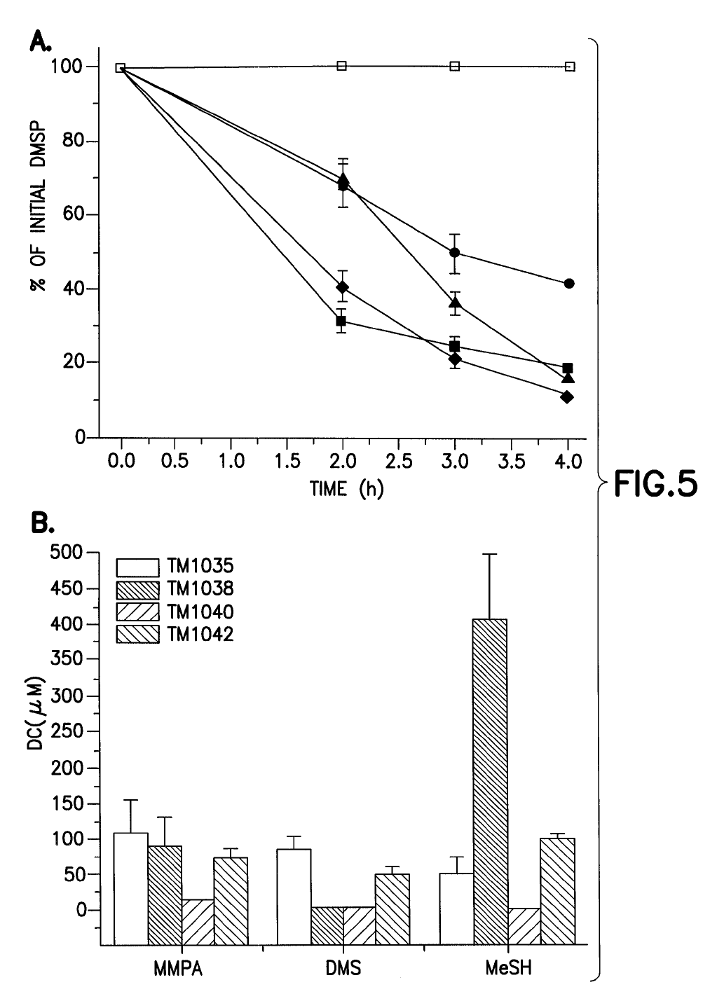

Using inocula from the P. piscicida CCMP1830 culture, nine bacterial strains were isolated, representing a range of colony phenotypes. An analysis of the rate of DMSP degradation showed that four of the nine strains degraded DMSP within a 4-h period, as shown in FIG. 5A while the other five showed little or no ability to degrade DMSP (data not shown). Strain TM1040 appeared to degrade DMSP fastest, followed by strains TM1035 and TM1042, with strain TM1038 being the slowest DMSP degrader. Three distinct colony morphologies were apparent in the four DMSP-degrading strains. Strains TM1035 and TM1042 were similar and produced small (1- to 1.5-mm-diameter), translucent, smooth colonies with a light-pink pigment. Strain TM1038 also produced light-pink colonies with a translucent appearance, but they were much smaller (0.2- to 0.7-mm diameter). TM1040, on the other hand, gave rise to colonies that were larger (4- to 6-mm diameter), translucent, and smooth with a brownish-yellow pigment that diffused throughout the agar medium.

The production of DMSP catabolites by each of the four bacterial isolates was assessed 3 h after addition of exogenous DMSP, as shown in FIG. 5B. All four strains produced the primary demethylation product of DMSP, MMPA, while three strains (TM1035, TM1038, and TM1042) also produced the secondary demethiolation product, MeSH. Among the three MeSH-producing strains, TM1038 produced significantly more MeSH, while TM1035 and TM1042, but not TM1038, produced DMS from DMSP in addition to MMPA. These data indicate that TM1035 and TM1042 possess both the DMSP lyase and demethylation pathways.

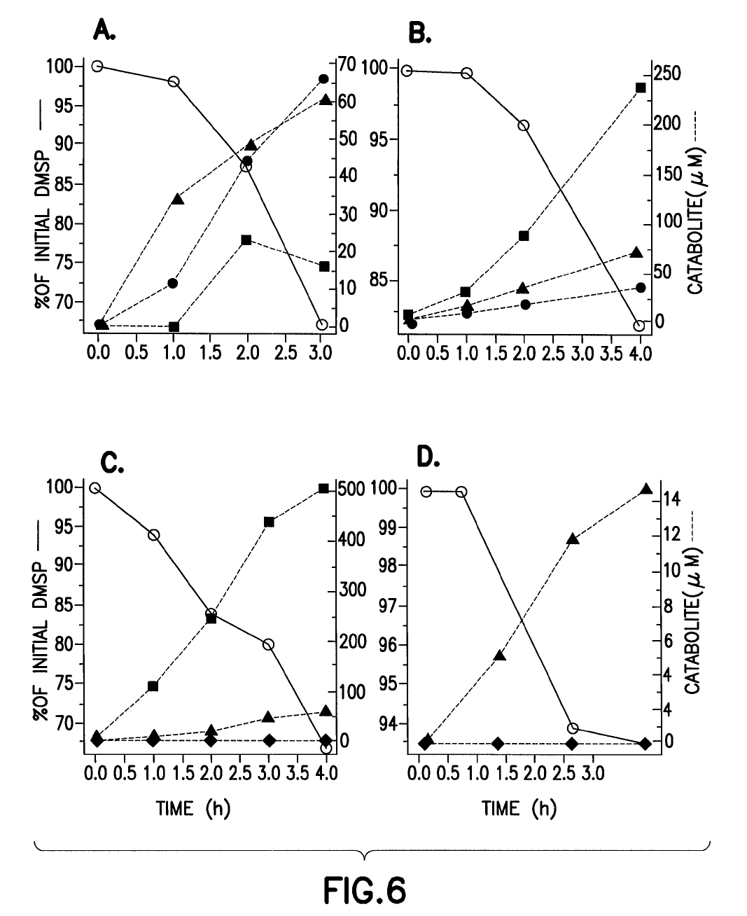

The kinetics of DMSP degradation and catabolite production were examined in greater detail and the results are shown in FIG. 6. Strains TM1035 and TM1042 have similar colony morphologies (medium size; pink), suggesting that they may be taxonomically closely related. The two strains also produce some of the same DMSP catabolites; however, they differ in both the

TABLE 2
Rates of DMSP metabolism of four bacterial strains
isolated from P. piscicida CCMP1830 culture.

| | | | | Rate of DMSP |
|---|---|---|---|---|
| | Rate of production (pM/h)^{a} | | | degradation |
| Strain | MM | MeSH | DMS | (pM/h)^{a} |
| TM1035 | 19.5 | NA^{b} | 22.7 | 95 |
| TM1038 | 15.9 | 132 | NP^{c} | 22.3 |
| TM1040 | 5.1 | NP | NP | 91 |
| TM1042 | 17.9 | 9.1 | 59 | 32.6 |

^{a}Rates of DMSP degradation an production of DMSP catabolites calculated based upon the slope of the linear portion of each came from FIG. 6.
^{b}NA p < 0.90.
^{c}NP. catabolite not detected or produced by this strain.

rate of DMSP degradation and the rate of production of DMS. Strain TM1035 removed ˜33% (330 μM) of the added DMSP during a 3-h period and produced ˜65 μM DMS, 60 μM MMPA, and 16 μM MeSH as a result. Production of DMS and MMPA occurred within 30 min, with MeSH production following 1 h later. On the other hand, strain TM1042 removed 19% (190 μM) of the added DMSP during the same 3-h period, producing 236 μM MeSH, 38 μM DMS, and 73 μM MMPA, FIG. 6B. Production of these compounds occurred simultaneously and within 30 min. The first-order rate constants for DMSP degradation and catabolite production were calculated from linear regions of FIG. 6 and are presented in Table 2.

TM1035 and TM1042 produced MMPA at similar rates (19.5 and 17.5 μM per h, respectively). MeSH production by strain TM1035 was highly variable across replicate experiments. Therefore, it was not possible to compare the rates of MeSH production in these two strains, although TM1042 always produced higher levels of MeSH as shown in FIG. 5B. TM1042 also had a lower rate of DMSP degradation (32.6 μM per h) than TM1035 (95 μM per h) and a higher rate of DMS production (59.0 μM per h) than TM1035 (22.7 μM per h). These data suggest that, while they produce similar colony phenotypes, TM1035 and TM1042 are physiologically unique, at least in their metabolism of DMSP.

Strain TM1038 produced both the demethylation and demethiolation products MMPA and MeSH, respectively, but no products of the lyase pathway. The strain removed ˜33% (330 μM) DMSP during a 4-h period, producing 502 μM MeSH and 61 μM MMPA, as shown in FIG. 6C. Of the four strains, TM1038 was slowest in degrading DMSP (22.3 μM per h) but had the highest rate of MeSH production (132 μM per h), which was at least an order of magnitude above that of TM1042. The rate of production of MMPA by TM1038 (15.9 μM per h) was quite similar to those of TM1035 (19.5 μM per h) and TM1042 (17.5 μM per h).

The fourth strain, TM1040, catabolized DMSP to produce only MMPA. The cells removed ˜7% (70 μM) DMSP in a 3-h period while producing only 15 μM MMPA (FIG. 6D). Thus, strain TM1040 has a high rate of DMSP degradation (91 μM per h), but a very low rate of MMPA production (5.1 μM/h), compared to the other three DMSP-degrading strains (Table 2).

### Taxonomic Identification of DMSP-Degrading Bacteria.

A taxonomic analysis of the DMSP-metabolizing strains placed the four isolates in the α-Proteobacteria, closely related to the Roseobacter clade, as shown in FIG. 7. Strains TM1042 and TM1035 are closely related to each other (99% identity; 1,306 of 1,310 bp) and cluster with another dinoflagellate-associated bacterium, strain ATAM407_{—}61, isolated from the dinoflagellate Alexandrium lustanicum (Hold et al., 2001). Both TM1035 and TM1042 are also more distantly related to R. tolerans from Ekho Lake (96% [1,246 of 1,294 bp] and 96% [1,248 of 1,294 bp] identity, respectively), which, like strains TM1035 and TM1042, is capable of producing both DMS and MeSH from DMSP (Gonzalez et al., 2003).

The sequences of TM1038 and TM1040 16S rDNAs show the greatest similarity to 16S rDNA sequences obtained from bacteria within the Roseobacter clade, yet they did not cluster well with any cultured organisms within this clade and are unrelated to any of the well-characterized Roseobacter species listed in GenBank. As shown in FIG. 7, the 16S rDNA from TM1038 was 95% (1,230 of 1,282 bp) homologous to rDNA obtained from an uncharacterized bacterium isolated from marine snow, HP29w (Gram et al., 2002). Similarly, strain TM1040 grouped with an uncharacterized α-proteobacterium, MBIC1887 (99% sequence identity; 1,275 of 1,284 bp), and to a lesser extent showed some relatedness to two well-characterized Silicibacter species, S. pomeroyi and S. lacuscaerulensis. Both of these Silicibacter species are capable of DMSP degradation (Gonzalez et al., 2003).

### Example 2

### Chemotaxis of Silicibacter sp. Strain TM1040

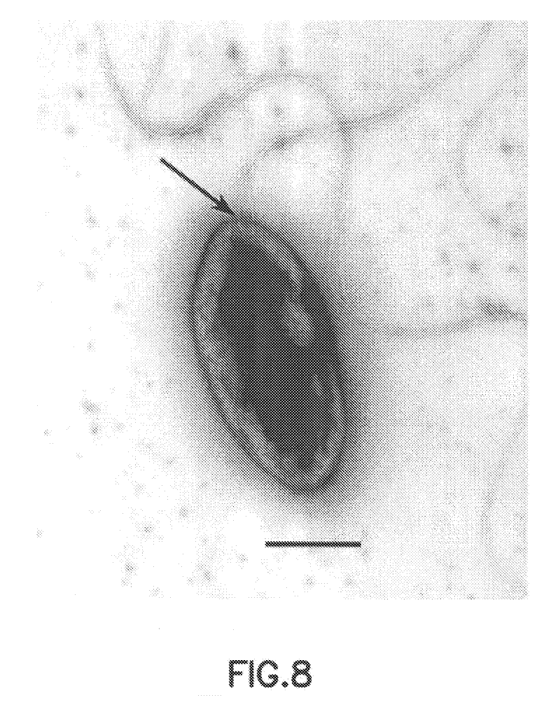

Next, it was determined and discussed the mechanics of how and why the Silicibacter sp. strain TM1040 maintains its interaction with P. piscicida. TM1040 possesses three lophotrichous flagella (shown in FIG. 8) and is highly motile, leading to an understanding that TM1040 interacts with P. piscicida through a chemotactic response to compounds produced by its dinoflagellate host. A combination of a rapid chemotaxis screening assay and a quantitative capillary assay were used to measure chemotaxis of TM1040. To help identify the essential attractant molecules within the homogenates, a series of pure compounds were tested for their ability to serve as attractants. The results show that TM1040 is strongly attracted to amino acids and DMSP metabolites, while being only mildly responsive to sugars and the tricarboxylic acid cycle intermediates. Adding pure DMSP, methionine, or valine to the chemotaxis buffer resulted in a decreased response to the homogenates, indicating that exogenous addition of these chemicals blocks chemotaxis and suggesting that DMSP and amino acids are essential attractant molecules in the dinoflagellate homogenates.

### Bacteria and Media.

Silicibacter sp. strain TM1040 was isolated from a culture of the dinoflagellate P. piscicida CCMP1830 (Provasoli-Guillard National Center for Culture of Marine Phytoplankton) and was maintained on either HIASW agar (25 g of heart infusion broth [Difco], 15 g of artificial seawater [ASW; Instant Ocean], 16 g of Bacto Agar per liter) or half-strength 2216 marine agar. Liquid broth cultures were made with half-strength 2216 marine broth. Marine motility agar was prepared by supplementing half-strength 2216 marine broth with 3.0 g of Bacto Agar per liter. For the chemotaxis plate assay, a basal minimal (BM) medium (12.1 g of Tris HCl, 1.0 g of NH₄Cl, 0.0075 g of K₂HPO₄, 15 g of ASW, 3.0 g of Bacto Agar per liter; pH 7.6) was used. After autoclaving, the BM medium was cooled to 50° C. and supplemented with 0.2 g of FeSO₃ and 1 ml of Balch's vitamins, and a single carbon source (glycerol, glucose, succinate, or alanine) was added to a final concentration of 10 mM.

### Dinoflagellates and Cultivation.

P. piscicida CCMP1830 was grown as previously described (Alavi et al. 2001). Dinoflagellates were fed a diet of the cryptomonad prey alga Rhodomonas sp. CCMP768, supplied as described by Alavi et al., 2001.

### Chemotaxis Plate Screening Assay.

A qualitative chemotaxis screening method, described by DeLoney-Marino et al. (2003), was used with only slight modification. An isolated colony of TM1040 was inoculated in the center of a BM-glycerol motility agar plate. After 3 days, a point where the bacteria had swam outwards 5 cm from the site of inoculation, an inoculum taken from the outer ring of the motile colony was used to inoculate the center of a second fresh BM-glycerol motility agar plate. Following 36 h of incubation, a putative attractant was placed approximately 5 cm from the site of inoculation (or 2.5 cm in front of the periphery of motile cells). Possible chemoattractant compounds were administered as either a sterile solid or concentrated stock solutions. The cells were further incubated for 16 h to allow for additional outward movement, at which time measurement of diameter, shape, and chemotaxis rings internal to the motile colony were made. The resulting data were scored on a plus-minus scale, where minus indicates no change compared to distilled water or no attractant controls and either one or two pluses indicates moderate to strong alteration in motile colony phenotype (respectively). All chemotaxis assays were performed in a 30° C. walk-in incubator at 65% relative humidity.

### Quantitative Capillary Chemotaxis Assay.

The capillary method of Adler (1966), as modified by Palleroni (1976), was used to quantitatively measure the chemotactic response of TM1040 toward a subset of compounds screened by the plate method. A broth culture of TM1040 was grown overnight in half-strength 2216 marine broth at 30° C. to an optical density at 600 nm (OD₆₀₀) of 0.3 to 0.4, which corresponds to the mid-exponential phase of the Silicibacter sp. strain TM1040 growth cycle. The cells were pelleted by centrifugation at 4,000×g in a tabletop centrifuge (Centra-CL2; International Equipment Company), the supernatant was discarded, and the pellet was resuspended in chemotaxis buffer (CB) (15 g of ASW, 6 g of Tris-HCl per liter [pH 7.6]) to the same OD. Five hundred microliters of washed cells was placed in one of the wells of the Palleroni chamber, followed by a 1-μl capillary (Microcaps; Drummond) filled with a putative attractant diluted in CB. This process was repeated for each of the four wells in the chamber. A capillary filled with CB only was included in each experiment as a negative control. Depending on the experiment, capillaries remained in the chambers for 0.5 to 2 h, after which time they were removed, and the contents of each capillary were serially diluted in CB. Final dilutions were then spread on HIASW agar plates and incubated for 16 h at 30° C. The concentration of TM1040 in each capillary was derived by counting the CFU on each plate and multiplying by the appropriate dilution factor. To normalize all data, a “response factor” as described by Wei and Bauer (Wei et al., 1998) was calculated by dividing the mean number of bacteria in the attractant-filled capillaries by the mean number of bacteria in control capillaries.

The capillary assay was also used to measure the reduction in chemotaxis through competition when a known attractant was supplied exogenously (i.e., outside the capillary). In this variation, the quantitative capillary method was used, but a known attractant was included in the CB used to suspend the washed cells. A capillary containing a mixture of potential chemoattractant molecules was then placed in the chamber and incubated as described earlier. At the completion of the assay, the percent reduction in chemotaxis was calculated using the following formula: % reduction=[1−(CFU ml⁻¹_{exogenous}/CFU ml⁻¹_{control})]×(100).

### Preparation of Cell Homogenates.

Dinoflagellates and Rhodomonas sp. were grown to late exponential phase (ca. 10⁵ to 10⁶ cells per ml), and the cell density was measured using a Coulter Counter (model M/SZRII; Beckman Coulter). Dinoflagellates were harvested after starvation of the culture to remove Rhodomonas prey algae, which was confirmed by microscopy. The cell densities in P. piscicida and Rhodomonas cultures were normalized to 10⁵ cells per ml, and each culture was then pelleted by centrifugation at 4,000×g and 4° C. for 10 min. The supernatant was removed, and the pellets were separately resuspended with either 1 ml of ice-cold distilled water or CB. The cells were homogenized by sonication (Sonic Dismembrator; Fisher), and cell disruption was assessed by microscopy. To examine the heat sensitivity of chemotaxis elicitors, an aliquot of each homogenate was heated to 80° C. for 15 min prior to the assay. All homogenates were then stored at −20° C. When required, the homogenates were thawed, held on ice, and used in the capillary assay as described.

Bacterial homogenates were prepared in a similar manner. Briefly, an aliquot from several 10-fold dilutions of the P. piscicida culture was inoculated onto half-strength 2216 marine agar and incubated until bacterial colonies were evident. Approximately 1,000 mixed colonies were then resuspended from the agar surface using 10 ml of CB, and the cells were collected by centrifugation at 4,000×g for 10 min. The bacterial pellet was resuspended to an OD₆₀₀ of 0.3 in CB. Aliquots of the bacterial suspension were sonicated, and a portion was heated, as described above.

### Video Microscopy.

To compare the motility of TM1040 with and without an attractant, cells were grown and prepared as described for in the capillary assay. A sample of the washed cells was incubated at 30° C., with and without the addition of 10 mM succinate (in CB, pH 7.0). Cell swimming and behavior over a 5-h period were measured using phase-contrast microscopy (Nikon Optiphot BX60) equipped with a digital video camera (Canon Elura, Lake Success, N.Y.). After examination of the entire video, three separate fields were chosen, and 1-min intervals of each were transferred to a computer for further analysis, using Adobe (San Jose, Calif.) Premier version 6.0 software. Individual 0.33-s frames were exported as a tagged image file format sequence and reassembled with IPLab version 3.55 (Scanalytics, Fairfax, Va.) as a sequence of ordered frames (t-series). The numbers of motile and nonmotile cells per field were determined to determine the average percentage of motile cells per field.

Transmission Electron Microscopy (TEM).

Silicibacter sp. strain TM1040, taken from the periphery of a motile colony growing in semisolid BM glycerol motility agar, was inoculated in half-strength 2216 marine broth and incubated at 30° C. to an OD₆₀₀ of 0.3. A 400-μm-mesh carbon-coated parlodion copper grid was floated over a 30-μl aliquot of this culture for 1 to 2 min and blotted dry. Bacteria adhering to the grid were stained two times for 30 s with 1% uranyl acetate and 0.04% tylose in distilled water. Negatively stained cells were viewed using a Philips BioTwin CM120 transmission electron microscope at an operating voltage of 20 kV. The resulting images were recorded on film and scanned into a computer, and the brightness and contrast were changed for optimum viewing using Adobe Photoshop 7.

### Analytical Techniques.

The concentration of DMSP in 1 ml-samples of heated or boiled 200 μM DMSP and in heated and untreated P. piscicida homogenates was measured using gas chromatography with flame ionization detection, as previously described.

### Chemicals.

DMSP was synthesized from acrylate and dimethylsulfide as previously described.

### TM1040 Possesses Three Lophotrichous Flagella.

Prior to beginning studies of chemotaxis, motile cells of TM1040 were visualized by TEM to confirm the presence, number, and location of flagella, as shown in FIG. 8. TM1040 is a small (ca. 1.0- to 1.5-μm) rod- or oval-shaped bacterium with at least three lophotrichous flagella. These flagella are located at one end of the cell, slightly off-center from the cell pole. An analysis of the structure of filaments suggests that they are simple filaments, rather than the complex forms found in Silicibacter pomeroyi DSS-3 (González et al., 2003) or other α-proteobacteria (Schmitt et al., 1974).

### Chemotaxis of TM1040 is Enhanced by Prior Starvation.

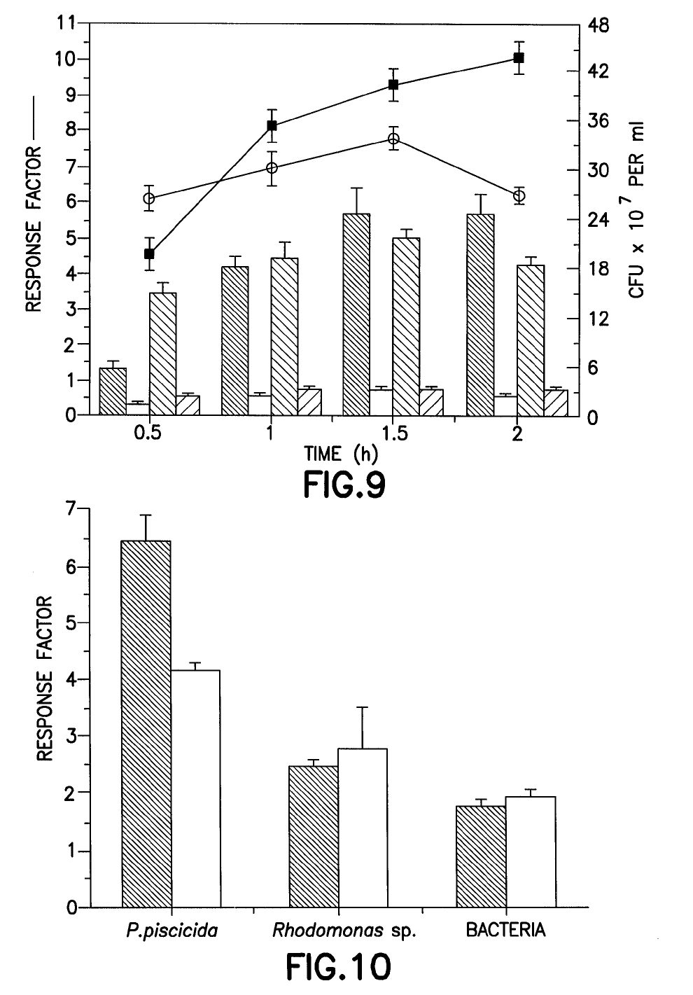

Initial observations suggested that TM1040 cells held for 1 to 2 h in CB (a starvation condition) were more responsive to methionine than cells that had not been starved (data not shown). To examine this more closely, chemotaxis of starved and unstarved TM1040 toward methionine was measured by using a quantitative capillary assay. As shown in FIG. 9, starving the bacteria of nutrients prior to the assay resulted in a greater maximal response to the attractant, which was maintained for at least 2 h. This can be seen by comparing the starved-cell response to methionine to the unstarved response to methionine in FIG. 9. After 0.5 h, the mean concentration of bacteria in methionine-filled capillaries was 5.3×10⁷ CFU per ml and increased thereafter, with increasing exposure time rising from 1.8×10⁸ CFU per ml at 1 h to a maximum of 2.4×10⁸ CFU per ml at 1.5 and 2 h. By contrast, without starvation the maximum mean concentration of bacteria in methionine-filled capillaries reached a peak of 2.1×10⁸ CFU per ml at 1.5 h and decreased thereafter. The bacterial concentration in control capillaries for either starved or unstarved cells reached an equilibrium of 2×10⁷ CFU per ml after 0.5 h.

The difference between the chemotactic responses of starved and unstarved cells is also shown in FIG. 9. The response factor is calculated by dividing the concentration of bacteria in attractant-filled capillaries by the concentration of bacteria in control capillaries (Wei et al., 1998). The response factor of starved cells to methionine is higher than the response of unstarved cells at all times except 0.5 h (where equilibrium has yet to be reached). Significantly, a comparison of the response factors of starved cells and unstarved cell confirms that starvation improves the chemotactic response of TM1040 and maintains it longer.

### Motility is Affected by Starvation.

The motility of the starved TM1040 cells was measured by light microscopy. While starvation enhanced the chemotactic response, it also reduced swimming motility, and prolonged starvation resulted in a majority of the cells becoming nonmotile. After 2 h, the percentage of motile cells under starvation conditions decreased drastically to a mean of 5.93% (n=69), and by 4 h all of the cells were nonmotile (n=66). In comparison, the percentage of motile cells under nonstarvation conditions did not change after 5 h and was similar to the percentage of motile cells after 1 h (mean, 26.64%; n=69). Thus, while starvation stimulates chemotaxis, it also depletes the energy supplies required to rotate the flagella. Based on these results, a starvation period of 1.5 h was considered optimal and was used in all further capillary assays.

### TM1040 is Attracted to Dinoflagellate Homogenates.

Does TM1040 sense and respond to dinoflagellates? To determine this, chemotaxis of TM1040 toward cell homogenates of P. piscicida and other constituents of the dinoflagellate culture was measured using the capillary assay. It is important to emphasize that the P. piscicida culture normally contains three different types of organisms: the dinoflagellates, the prey algae (Rhodomonas sp.), and a diverse bacterial population that includes TM1040. Rhodomonas can be virtually eliminated from the cultures through attrition from dinoflagellate feeding, but the bacterial community cannot be removed without adversely affecting the dinoflagellates themselves. So, a strategy was developed to measure the chemotaxis of TM1040 towards three cell homogenates: dinoflagellates plus associated bacteria, Rhodomonas, and a mixture of heterotrophic bacteria obtained from the same dinoflagellate culture. In this manner, the relative contribution of each population towards eliciting a chemotactic response from TM1040 could be assessed.

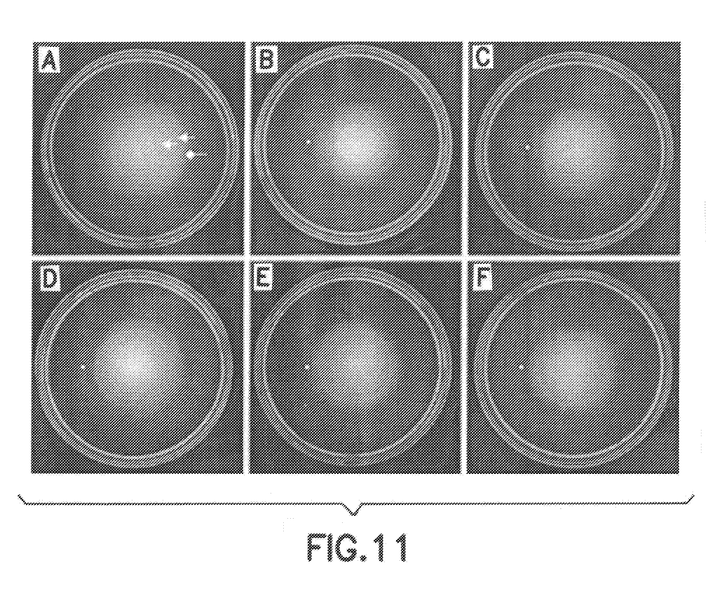

As is shown in FIG. 10, TM1040 responded strongly to P. piscicida homogenates, producing a response factor of 6.4. This response is mediated in part by heat-labile compounds because heating of the same homogenates prior to the assay reduced the response by 36% (response factor=4.1). TM1040 is substantially less chemotactic to Rhodomonas homogenates, and this response did not change upon heating of the algal homogenate, remaining at a mean of ca. 2.5. Equally, bacterial homogenates were also poor elicitors of TM1040 chemotaxis, giving a mean response factor of only 1.9 and 1.7, with or without heating, respectively. These results indicate that TM1040 is more strongly attracted to components present in P. piscicida cell homogenates, some of which are heat labile.

### DMSP Compounds and Amino Acids are Strong Chemoattractants of TM1040.

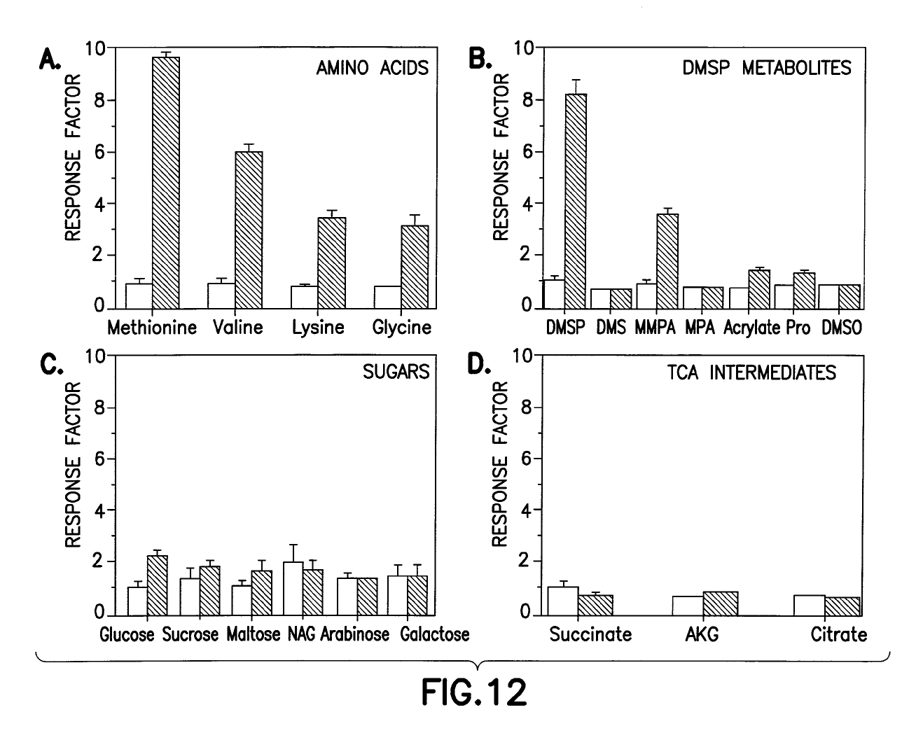

A chemotaxis plate assay (DeLoney-Marino et al., 2003) was used to screen a large number of pure compounds thought to be in dinoflagellate cell homogenates for their ability to affect the chemotactic behavior of TM1040. This assay utilizes a minimal medium with 0.3% agar (BM glycerol motility agar) that allows the bacteria to swim through the agar matrix. Bacteria inoculated into the center of the agar consume nutrients and create a concentration gradient that increases outward from the point of inoculation. The bacteria sense the increasing gradient and swim outwards, seeking higher concentrations of nutrients, which is manifested as a symmetrical “motile” colony. If an attractant is placed at a short distance in front of the advancing motile colony, it forms a second gradient moving towards the oncoming cells that will affect the symmetry of the colony by reducing the net outward swimming of the bacteria. This is shown in FIG. 11. When inoculated in BM glycerol motility agar (FIG. 11A), TM1040 cells swim outward from the point of inoculation to form a colony that often contains one or two internal bands of cells. These bands have been associated with subpopulations of bacteria that are responding to different attractants (Wolfe et al., 1989).

As measured by this method, TM1040 is chemotactically responsive to a number of different chemicals, most notably DMSP and its catabolites, as well as amino acids. For example, addition of DMSP (FIG. 11B) caused a marked deformation in the periphery of the motile colony, highlighted by flare of bacteria and disruption of the outer ring. Responses to methionine (FIG. 11C) and valine (FIG. 11D) also had significant effects on the periphery of the motile colony. In comparison, there was no detectable change in the appearance of the motile colony when either arabinose (FIG. 11E) or fructose (FIG. 11F) was tested, suggesting that these two sugars do not affect TM1040 chemotaxis.

The response to other chemicals was scored on a plus-minus scale. TM1040 is attracted to all amino acids, similar to the response observed with methionine or valine. The bacterium responded strongly to the DMSP metabolites, acrylate and MMPA, while only weakly to MPA. These responses were similar to that of methionine and valine. Of the 12 sugars that were tested, TM1040 responded positively to (in order of response) sucrose, N-acetylglucosamine (NAG), galactose, glucose, and maltose. The tricarboxylic acid cycle (TCA) intermediates, citrate and fumarate, also elicited a strong positive response from TM1040, while only a mild response to succinate and no response to α-ketoglutarate was seen as shown in Table 3.

TABLE 3
Change in motile colony morphology of TM1040 in
response to attractants, compared to the control

| | |
|---|---|
| Attractant(s) | Response^{a} |

Amino acids

| | |
|---|---|
| Alanine, arginine, asparagine, aspartic acid, glutamic acid, | ++ |
| methionine, phenylalanine, proline, threonine, valine | |
| Glycine, histidine, cysteine, isoleucine, leucine, lysine, serine, | + |
| tryptophan, tyrosine | |

DMSP metabolite(s)

| | |
|---|---|
| DMSP, acrylate, MMPA | ++ |
| MPA | + |
| DMSO | − |

Sugars

| | |
|---|---|
| Galactose, N-acetylglucosamine, sucrose | ++ |
| Glucose, maltose, glycerol | + |
| Lactose, arabinose, fructose, fucose, mannose, ribose, xylose | − |

TCA Intermediate(s)

| | |
|---|---|
| Citrate, fumurate | ++ |
| Succinate | + |
| Alpha-ketoglutarate | − |

^{a}The strength of the response indicates the change in motile colony morphology when a chemical is spotted near the colony periphery compared to the control, no spotted chemical.

As described, BM glycerol medium was used because TM1040 can utilize glycerol as a sole carbon source, as has been observed for other roseobacters (Shiba 1991). However, it is known that chemotaxis behavior in other bacterial species can be affected by the availability of background nutrients. Indeed, when glucose was used in place of glycerol as the sole background carbon source, TM1040 chemotaxis was severely affected, and many of the chemicals that were attractants using BM glycerol failed to produce an effect in BM glucose as shown in Table 4. The two exceptions were acrylate and succinate, which both produced a moderate response in the glucose background.

TABLE 4
Change in motile colony morphology of TM1040 in
response to putative attractants when grown in
BM motility agar with different carbon sources

| | Change in morphology with carbon source^{a} | | | |
|---|---|---|---|---|
| Chemical | Glycerol | Glucose | Succinate | Alanine |
| Acrylate | ++ | + | ++ | + |
| Alanine | ++ | − | + | − |
| Alpha-ketoglutarate | − | − | + | − |
| DMSO | − | − | − | − |
| DMSP | ++ | − | + | + |
| Galactose | ++ | − | + | − |
| Glucose | + | − | + | + |
| Glutamic Acid | ++ | − | + | − |
| Lactose | − | − | − | − |
| Lysine | + | − | − | − |
| Maltose | + | − | ++ | ++ |
| N-acetylglucosamine | ++ | − | + | + |
| Succinate | + | + | − | ++ |
| Valine | ++ | − | + | + |

^{a}TM1040 was inoculated into the center of a BM motility agar plate containing a single carbon source (glycerol, glucose, succinate or alanine) and allowed to grow and move outwards. Chemicals were then spotted near the periphery of the motile colony, and the change in colony appearance compared to the control (no spotted chemical) was recorded on a plus/minus scale.

In contrast to glucose, when either succinate or alanine was used as the sole carbon source, TM1040 chemotactic behavior was similar to what had been observed in BM glycerol medium, albeit the response was often less intense. Not surprisingly, since chemotaxis relies on the establishment of chemical gradients, TM1040 did not respond to any chemical when the same chemical was incorporated into the BM motility medium.

### Quantitative Chemotaxis Toward Pure Compounds.

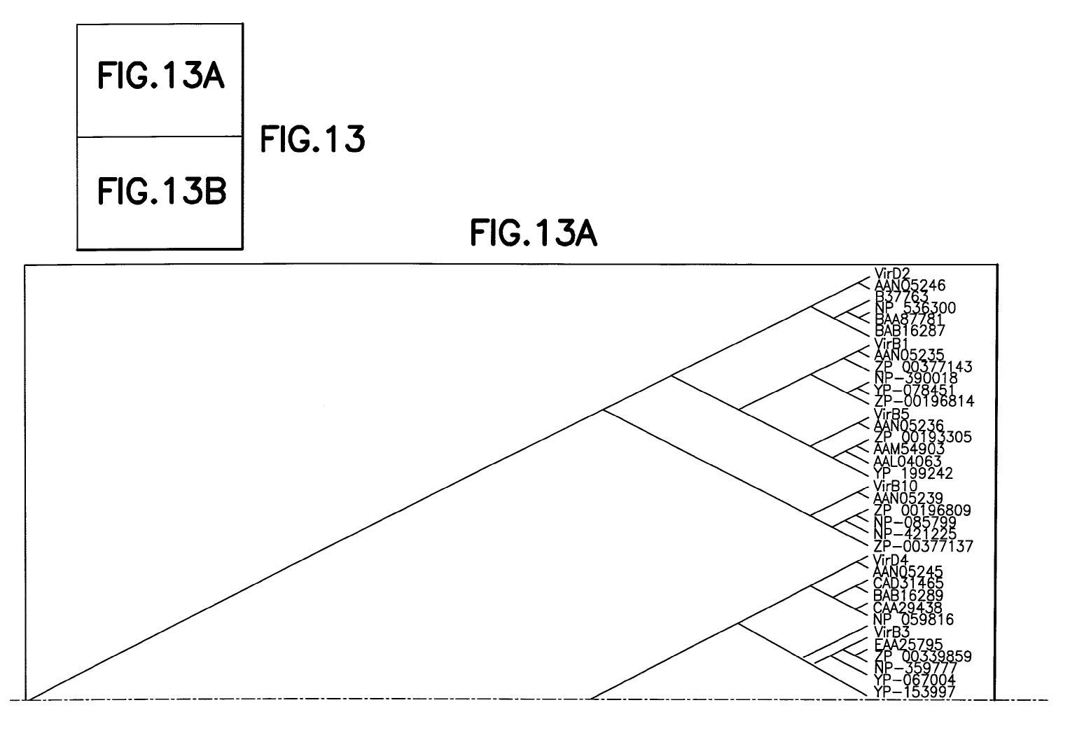

The results of the chemotaxis screening assay were used to select a subset of chemicals for further testing, using a quantitative capillary assay. As the data in FIG. 12 show, the response of TM1040 to a given attractant is concentration dependent, e.g., concentrations below 2 μM failed to elicit a significant response, while a 200 μM concentration of an attractant produce strong chemotaxis (FIG. 12A). Among the amino acids tested, methionine and valine gave the strongest response factors (means of 9.8 and 6.1, respectively), while lysine and glycine produced a similar response (mean, ca. 3.5) from TM1040. TM1040 also showed strong chemotaxis toward DMSP, which produced a response factor of 8.2 (FIG. 12B). The only breakdown product of DMSP that is a significant chemoattractant for TM1040 is MMPA (mean response factor of 3.8). In comparison, all the sugars tested produced only a weak to mild chemotactic response from TM1040 (means of <3 [FIG. 12C]). Of these sugars, TM1040 was most attracted to glucose, producing a mean response factor of 2.1. FIG. 12 also shows the response of TM1040 to TCA intermediates. Of the three TCA intermediates that were tested (succinate, α-ketoglutarate, and citrate), none were found to be significant chemoattractants for TM1040, suggesting that these chemicals play a minor role in the overall response of TM1040 toward dinoflagellates. Overall, the results from the capillary assay agree with the data obtained from the qualitative assay using BM motility agar.

Chemotaxis Toward P. piscicida Homogenates is Inhibited by Externally Supplied Attractants.

As was noted in the data presented in Table 4, in the presence of an externally supplied attractant distributed homogeneously throughout the medium, chemotaxis toward a point source of the same attractant is inhibited because the gradient is deflated.

Using this knowledge, experiments were designed to identify the heat-labile attractants found in the dinoflagellate homogenates. As shown in Table 5 and in accord with the data in FIG. 10, the response of TM1040 to untreated dinoflagellate homogenate in the absence of any externally supplied attractant was greater than its response when the homogenate was preheated to 80° C. This relationship was altered, however, when DMSP, methionine, or valine was supplied externally in the buffer. In these cases, the mean number of bacteria in the untreated capillaries was 3.33×10⁴, 3.03×10⁴, or 1.53×10⁴ CFU per ml, giving a reduction in chemotaxis of 54, 58, or 78%, respectively (Table 5). An externally added attractant also reduced TM1040 chemotaxis to heated homogenates, with external valine producing the greatest reduction (74%), followed by methionine (48%) and DMSP (11%).

TABLE 5
Reduction in chemotaxis of TM1040 toward untreated and heated P. piscicida
homogenates by external addition of known attractants

| | No. of bacteria per capillary (CFU · 10⁴)^{b} with: | | | | | | |
|---|---|---|---|---|---|---|---|
| | | Attractant added to external buffer^{c} | | | | | |
| Treatment^{a} | Control^{d} | DMSP | % Reduction^{e} | Methionine | % Reduction | Valine | % Reduction |
| None | 7.24 (±1.39) | 3.33 (±0.15) | 54.01 | 3.03 (±0.32) | 58.15 | 1.53 (±0.15) | 78.87 |
| Heat | 3.51 (±0.35) | 3.10 (±0.16) | 11.68 | 1.80 (±0.11) | 48.72 | 0.91 (±0.24) | 74.07 |

^{a}Chemotaxis of TM1040 toward heated (80° C. for 15 min) or untreated P. piscicida homogenates was measured using the capillary assay (see Materials and Methods).
^{b}Standard deviation is given in parentheses.
^{c}Prior to the assay, DMSP, methionine, or valine was added to external buffer containing motile cells of TM1040 at a final concentration of 200 μM and mixed to homogeneity.
^{d}The control was buffer only.
^{e}% Reduction, the percent reduction in CFU per capillary compared to the control.

DMSP in P. piscicida Homogenates is Destroyed by Heating.

These results suggest that while DMSP is an attractant in untreated homogenates, its attractant quality is reduced upon heating, possibly due to degradation of the molecule. Destruction of 200 μM DMSP in CB was assessed after heating the samples to either 80 or 100° C. for 15 min. At both temperatures, negligible (<2%) loss of DMSP was observed. These results indicate that pure DMSP is not a heat-labile molecule.

An alternative explanation for why heat reduced the chemoattractant quality of the homogenates is that heat acts indirectly through an intermediate to inactivate DMSP. This hypothesis predicts that the concentration of DMSP decreases when a homogenate is heated. The DMSP concentration in untreated and heated homogenates was measured. In untreated P. piscicida homogenates the mean concentration of DMSP was 8.21±1.2 μM. After the homogenate was heated, the concentration of DMSP fell below the level of detectability (<1 μM). These data indicate that heat-activated components of P. piscicida homogenates enhance the degradation of DMSP and may explain the reduction in chemotaxis of TM1040 toward heated homogenates.

### Example 3

The results of the present study show that Silicibacter sp. strain TM1040, originally isolated from P. piscicida dinoflagellate cultures, senses and responds chemotactically to compounds produced by the dinoflagellate cells. Thus, this represents the first report of chemotaxis behavior of a Roseobacter clade bacterial species. Since Roseobacter species are prevalent in marine environments and abundant within blooms of DMSP-producing phytoplankton, chemotaxis to DMSP by these bacteria is likely to be an important mechanism in establishing close interactions with the dinoflagellate at both the physical and physiological levels.

Many physiological characteristics of the Roseobacter clade of bacteria, such as Silicibacter sp. strain TM1040, make them well suited for life in close proximity to dinoflagellates and algal cells. The area immediately surrounding a dinoflagellate is a habitable niche for some marine bacteria, it is not surprising to find that a dinoflagellate-associated bacterium like TM1040 has mechanisms to exploit this niche. At present, these mechanisms include swimming motility and the chemotactic response of the cells to DMSP and other dinoflagellate molecules, as well as the enzymatic mechanisms to degrade and utilize DMSP. These physiological functions enhance the survival of these bacteria when associated with a dinoflagellate cell. Interestingly, a preliminary analysis of the genome of Silicibacter sp. strain TM1040 reveals several genetic loci that may serve to enhance the interaction of this bacterium with its dinoflagellate host.

Specifically, an analysis of the TM1040 genome revealed the presence of a complete complement of genes homologous to Agrobacterium tumefaciens vir genes whose function is involved in the transfer of DNA from the bacterium to plant cells. Thus, this discovery may allow the use of Silicibacter sp. TM1040 as a tool to deliver genes to marine algal species.

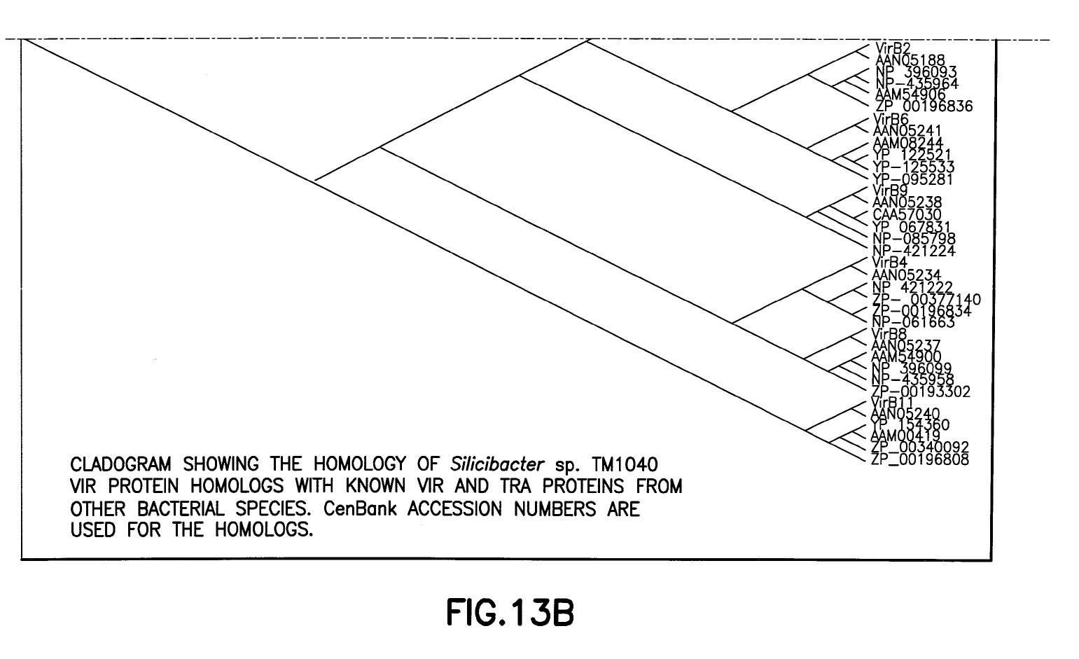

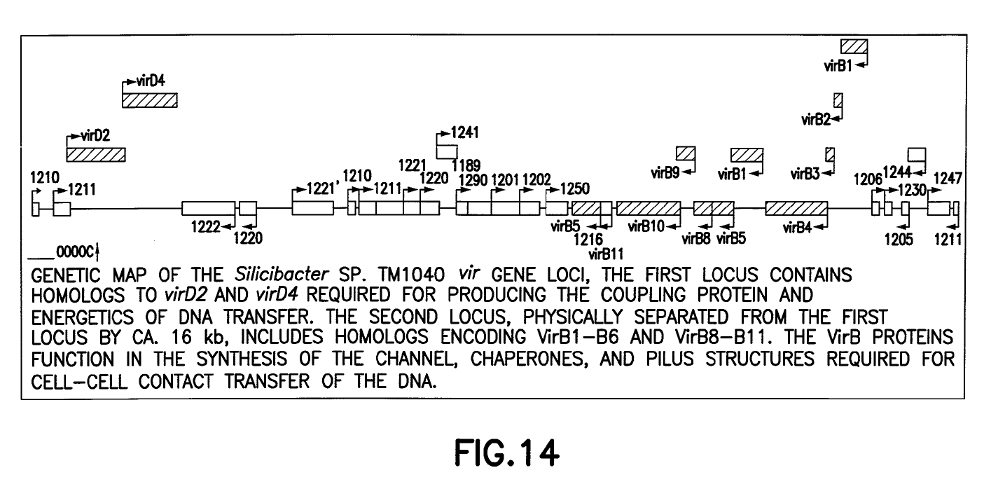

Strain TM1040 enhances the growth of P. piscicida through an unknown mechanism, but close physical interaction appears to be essential for the enhancement of dinoflagellate growth. As shown above, Silicibacter sp. TM1040 metabolizes the dinoflagellate secondary metabolite dimethylsulfoniopropionate (DMSP) and is chemotactically responsive to DMSP and other P. piscicida molecules. It is important to emphasize at this point that Silicibacter sp. TM1040 is the only member of the Roseobacter clade of bacteria that is known to form such a tight interaction with an algal species. An analysis of the genomic sequence of Silicibacter sp. TM1040, available at http://genome.jqi-psf.org/draft_microbes/siltm/siltm.home.html, http://img.jgi.doe.gov/v1.0/main.cqi?page=scaffolds&taxon_oid=400820000 or http://www.ncbi.nlm.nih.gov/entrez/guery.fcgi?db=Nucleotide&cmd=Search&term=AAFG01000001: AAFG01000020[PACC], the contents of which is hereby incorporated by reference herein for all purposes, indicates that the bacterium possesses a set of genes with strong homology to the Agrobacterium tumefaciens vir genes as shown in FIG. 13, which encode a cell-cell contact-mediated Type IV Secretory System (T4SS) and that are required for the transfer of A. tumefaciens T-DNA to plant cells (Christie et al., 2004 and 2001) (FIG. 14). Significantly, each of these genes is identical or nearly identical to homologs previously characterized on a plasmid, pSD25, isolated from another marine roseobacter referred to as Ruegeria isolate PR1b (Zhong et al., 2003). This is important because it was recently determined that Silicibacter sp. TM1040 harbors two plasmids (personal communication). Although the nature of those plasmids has not been fully disclosed, it is likely that the vir genes of TM1040 are also plasmid-borne. The first group consists of genes with homology to virD2 and virD4 (FIG. 14 and Table 6) which encode for the relaxase and coupling proteins providing the energetics for export of transfer DNA (T-DNA) (Cascales et al., 2004). The second group of 10 genes are homologous to A. tumefaciens virB1-2 B6 and virB8-B11 (Hapfelmeier et al., 2000). VirB7 is not present in the genome of Silicibacter sp. TM1040 and is considered a non-essential gene of Agrobacterium. The VirB proteins are responsible for producing the inner membrane channel and pilus structure (Cascales et al. 2004). In summary, all of the genes required to produce a functional cell-cell contact-mediated T4SS are present in the TM1040 genome. A BLAST search of the S. pomeroyii or Janasschia sp. CCS1 genomes using the VirD4 protein sequence from Silicibacter sp. TM1040, the most evolutionarily conserved component among type IV secretion systems, suggests that a T4SS is not present in these other Roseobacter species. As neither S. pomeroyi nor Jannaschia sp. CSS-1 form obligate interactions with algal species, the absence of vir genes in those species suggests that possession of the Vir homologs by Silicibacter sp. TM1040 represents a unique characteristic of the bacterium that is important for its interactions with algal hosts. Moreover, the strong homology between Silicibacter sp. TM1040 Vir proteins and their A. tumefaciens counterparts suggests that they potentially function to promote genetic exchange between strain TM1040 and its algal host cells (both micro- and macroalgal species).

The practical application of the knowledge of Silicibacter sp. TM1040 vir genes and T4SS lies in the potential to use the bacterium to genetically modify marine algae, both microalgal species (dinoflagellates, diatoms, and unicellular algae), as well as macro-algal species (commonly ‘seaweeds’). Marine algal species are widely used in businesses that include agriculture, cosmetics, food, pharmaceutical, and biotechnology companies. Marine algal species are used commercially to produce gelling agents (agar, alginate, carrageenan), cosmetics, pigments, and find applications in the food and beverage industries, and as pharmaceutical compounds (antimicrobial, anti-inflammatory, antitumoral, and antiviral). They are also finding application in the emergent neutraceutical (particularly in the production of anti-oxidants), botanicals and phytopharmaceutical industries.

Thus, a partial list of potential uses of Silicibacter sp. TM1040-mediated gene transfer includes the following applications:

1. Modify carotenoid-producing algae, i.e., Dunaliella salina, to over-produce beta-carotene.
2. Genetically engineer microalgal species to produce select essential omega-3 or omega-6 fatty acids, such as docosahexaenoic acid (DHA) or arachidonic acid (ARA), currently produced from a dinoflagellate (Crypthecodinium cohnii) and microalga (Schizochytrium sp.)
3. Alter the properties of nori (Porphyra sp.) or other edible macroalgae to enhance or modify coloration, texture, agar content, etc.
4. Modify the properties of carrageenan and agar from red seaweeds and alginic acid from brown seaweeds.
5. Genetically engineer marine algal to produce foreign proteins or compounds of interest to the pharmaceutical or chemical industries.
6. Enhance heavy-metal-binding properties of macroalgal for purposes of improved bioremediation of copper and other metal pollutants.

### Example 4

As discussed above, strain TM1040 is motile and swims towards dinoflagellate-derived molecules, such as dimethylsulfoniopropionate and amino acids, by chemotaxis. In the present study, mutants defective in swimming motility were used to determine the importance of bacterial flagella and swimming motility in the interaction between Silicibacter sp. TM1040 and P. piscicida. Silicibacter sp. TM1040 is actively motile by means of three lophotrichous flagella and is chemotactic towards DMSP, MMPA, and amino acids.

### Materials and Methods

### Bacteria and Media.

Silicibacter sp. TM1040 was grown in HIASW broth consisting of 25 g Difco Heart Infusion broth (Becton-Dickinson, Franklin Lakes, N.J.) supplemented with 10 g of artificial seasalts per liter (Instant Ocean, Aquarium Systems, Mentor, Ohio) or in halfstrength 2216 marine broth (Becton-Dickinson) as described by Alavi et al 2001. Marine motility agar was made by supplementing half-strength 2216 marine broth with 3.0 g of Bacto-agar per liter. Basal minimal (BM) broth containing glycerol as the sole carbon source was made according to the description described herein above. Escherichia coli DH5á \tilde{e} pir was grown in Luria-Bertani (LB) broth. Bacto-agar at 1.5% (w/v) was added to broths as required. As appropriate, kanamycin was used at 120 μg per ml for Silicibacter strains and 50 μg per ml for E. coli Dinoflagellates and cultivation.

P. piscicida CCMP1830 was grown as previously described in Example 2. Dinoflagellates were fed a diet of axenic Rhodomonas sp. CCMP768, using the method described by Alavi et al. 2001. All dinoflagellate culture manipulations were done in a laminar flow hood.

### Transposon Mutagenesis and Phenotypic Analyses.

Electrocompetent Silicibacter sp. TM1040 was prepared following the procedures of Garg with minor changes (Garg et al, 1999). Strain TM1040 was incubated in HIASW broth at 30° C. with shaking to an optical density at 600 nm (O.D.600) of 0.5. The cells were harvested by centrifugation at 8000×g for 10 min at 4° C., the supernatant was discarded, and the cell pellet washed four times in ice-cold distilled water. Following the final wash, the cell pellet was suspended in 10% glycerol and held on ice. A 50 μl aliquot of cells was mixed with 1 μl (25 ng) of the EZ::TN <R6Kãori/KAN-2> transposome (Epicentre, Madison, Wis.), and the mixture was electroporated at 16 kV per cm, yielding a time constant of ˜9.2 ms. The cells were suspended in 2 ml of pre-warmed HIASW broth and incubated 14-16 h at 30° C. with shaking. Samples of this culture were then spread onto HIASW agar containing kanamycin and incubated for 1-2 d at 30° C. The resulting kanamycin-resistant colonies were transferred to HIASW agar plus kanamycin and arranged in a 7-by-7 array to facilitate future analysis. Following incubation, the colonies were replicated on three media: fresh HIASW agar, marine motility agar to screen for motility defects, and BM plus glycerol to screen for auxotrophs. Motility (Mot−) mutants and auxotrophs were identified from the initial bank, picked to fresh media, and re-tested two more times to confirm the phenotype.

### Phenotypic Characterization of Mot-Mutants.

Mot-mutants were further analyzed for additional linked phenotypes. Since motility agar screening does not discriminate between defects in flagella or chemotaxis, each Mot-mutant was examined by phase-contrast microscopy for its ability to swim in HIASW, BM plus glycerol, and half-strength marine broths. The synthesis of flagella was determined using the flagellar silver staining protocol of West et al. (West et al., 1977). The length of individual cells was measured by analysis of phase-contrast images captured using a Quantix CCD camera (Photometrics, Tucson, Ariz.) and IPLab computer software (Scanalytics, Fairfax, Va.), with 5 μm beads (Ted Pella, Redding, Calif.) serving as a size reference. The ability of the mutants to form rosettes (groups of cells bound to each other at their cell poles, a characteristic of Silicibacter sp. TM1040 and other roseobacters (Ruger et al., 1992) was determined by light microscopic examination of cells grown in BM plus glycerol broth with shaking at 30° C. for 2 d. The growth rate of the Mot− mutants was determined by measuring the O.D.600 over a two-day period while incubating in HIASW broth at 30° C. with shaking.

### Cloning and Sequencing of EZ:TN Flanking DNA.

The EZ:TN transposon and flanking DNA were rescue-cloned by using the oriR6K origin of replication on the EZ:TN transposon. Genomic DNA from each Mot− mutant was extracted using phenol-chloroform extraction plus CTAB (cetyltrimethyl ammonium bromide) following standard protocols (Ausubel, F. M. 2001). Genomic DNA was digested with NcoI restriction endonuclease and treated with T4 DNA ligase to circularize the molecules. This was then transformed into E. coli DH5á ëpir by electroporation. The transformed cells were spread onto LB containing kanamycin and incubated at 37° C. overnight. Plasmid DNA was extracted from the resulting kanamycin-resistant colonies using a Qiagen Midi kit (Qiagen, Valencia, Calif.) according to the manufacturer's instructions. The nucleotide sequence of the DNA flanking the transposon of a representative plasmid from each rescue-cloning was obtained using two oligonucleotide primers, KAN-2 FP-1 and R6KAN-2 RP-1, as described by the manufacturer (Epicentre).

### Analysis of Transposon Insertion Site.

The site of transposon insertion in the genome of Silicibacter sp. TM1040 was determined through DNA:DNA homology searches using the DNA sequence flanking each transposon and the draft annotation of the Silicibacter sp. TM1040 genome provided by the Joint Genome Institute (JGI), U.S. Department of Energy (Walnut Creek, Calif.; http://genome.ornl.gov/microbial/rose_tm1040/). The genome sequence is deposited in GenBank (Bethesda, Md.) under accession number NZ_AAFG00000000. Nucleotide sequences flanking the transposon were aligned with the draft TM1040 genome sequence using BLASTN to identify the mutated gene (Altschul et al., 1990). Open reading frames (ORFs) were identified using the GeneMark program (Lukashin et al., 1998) and a BLASTP alignment of the deduced amino acid sequence. Further characterization of the putative gene products was made through a search for protein family domains using the Simple Modular Architecture Research Tool (SMART 4.0) (Letunic et al., 2004).

### Fluorescent Staining of Living Bacteria.

Silicibacter strains were stained with 5-(and 6-) carboxyfluorescein diacetate succinimidyl ester (CFDA/SE, Molecular Probes, Eugene, Oreg.) fluorescent tracer dye according to the method of Fuller et al., 2000. A 5 ml aliquot of half-strength marine broth was inoculated with either one of three Mot-mutants or the wild-type cells, taking as an inoculum cells growing at the periphery of a 2-day-old colony grown in marine motility agar. After overnight incubation at 30° C., the cells were pelleted by centrifugation at 4,000×g for 10 min. at room temperature, and suspended in 10 parts per thousand (ppt) artificial seawater (ASW; Instant Ocean) containing 1 mM succinate to an O.D.600 of 0.6. A 10 μl aliquot of 4 mM CFDA/SE tracer dye dissolved in DMSO was added to 1 ml of cells and the tubes inverted several times to mix. After 4 h staining in the dark, the cells were washed 3× with 10 ppt ASW at 8000×g for 10 min to remove unbound stain. After staining and prior to the final washing step a sample of stained wild-type cells was killed by heating at 65° C. for 10 min.

Measurement of Attachment of Silicibacter sp. TM1040 to P. piscicida.

Dinoflagellates were prepared by behavioral washing using the method described by Alavi et al 2001 with the following modifications. A 10 ml aliquot of P. piscicida culture at ˜105 cells per ml was added to a 14 ml polypropylene test tube (Falcon #352059) and allowed to remain undisturbed for 30 min. During this time dinoflagellates actively swim to the bottom of the container. 1 ml of the turbid bottom layer containing the dinoflagellate zoospores was carefully removed from the tube and added to 10 ml of sterile 10 ppt ASW in a separate tube. This process was repeated three more times, removing the majority of the unattached or free-living bacteria from the dinoflagellates. The washed dinoflagellates (1 ml sample) were added to 1 ml of 10 ppt ASW contained in a well of a tissue culture plate (Falcon 353046, Becton Dickson). Samples of mutant, wildtype and heat-killed TM1040 cells stained with CFDA/SE were diluted in 10 ppt ASW to ca. 105 cells per ml and 20 μl of each dilution was added to 2 ml of the washed zoospores. After 2 h, 1 ml of each suspension was fixed with 2.5% paraformaldehyde (w/v final concentration) on ice for 15 min. The fixed cells were pelleted by centrifugation at 8,000×g for 10 min. at 4° C. and suspended in 100 μl of 10 ppt ASW. Fifty μl of the cells was placed on a clean glass slide and the cells allowed to adhere to the glass. After 10 min, excess fluid was removed and the cells were covered with 10 μl of Prolong anti-fade solution (Molecular Probes, Eugene, Oreg.) and a glass cover slip. The number of CFDA/SE-stained bacteria associated with individual P. piscicida zoospores was determined using confocal scanning laser microscopy (CSLM) and epifluorescence.

### Confocal Scanning Laser Microscopy.

Dinoflagellates and physically associated CFDA/SE stained Silicibacter strains were visualized with a Bio-Rad Radiance 2100 AGR-3Q laser scanning confocal microscope with argon, green He/Ne and red diode lasers (Biorad, Hercules, Calif.), with an excitation wavelength of 488 nm and detected using a 500-535 nm band pass filter. Optical Z-sections at 0.5 μm increments were obtained and composite images assembled as previously described (Alavi et al., 2001).

### Epifluorescence Microscopy.

CFDA/SE-stained samples were also visualized using an Olympus BX60 upright microscope equipped with filter cubes giving excitation and emission maxima of 480 and 525 nm, respectively (Chroma, Rockingham, Va.). Phase contrast and fluorescent images were captured and phase-contrast and epifluorescent micrographs of the same field merged using ImageJ software (Rasbad, NIH, MD) with the co-localization plug-in (Bourdoncle, Institut Jacques Monod, Paris).

### Bacterial Add-Back Experiments.

Axenic zoospores were produced using the method of Alavi et al., 2001, which typically produced between 1,000-2,000 axenic zoospores per 10 ml culture. Axenic zoospore cultures were fed Rhodomonas sp. algae at a ratio of dinoflagellate to alga of 1:800, a ratio empirically determined to provide maximal growth of P. piscicida under these conditions. Silicibacter sp. TM1040 and Mot− mutant strains were incubated in HIASW broth overnight at 30° C. with shaking and washed twice in 10 ppt ASW. Each culture was normalized to an O.D.600 of 0.2 and placed on ice prior to use. A sample of wild-type strain TM1040 culture was heat-killed at 65° C. for 10 min to serve as a negative control. A 50 μl sample of each bacterial suspension was added to 10 ml of the axenic zoospores with gentle mixing to give ca.104 bacteria per ml. Cultures were incubated at 20° C. on a 14:10 light:dark cycle for 9 d. A sample of each culture was taken on days 1, 3, 5, 7 and 9 for enumeration of dinoflagellate and prey algal cell densities, and samples taken on days 1 and 9 for the analysis of bacterial species by denaturing gradient gel electrophoresis (DGGE).

### Cell Counts.

P. piscicida and Rhodomonas sp. were fixed in 10% (v/v) Bouin's fixative (Sigma, St. Louis, Mo.), diluted in 10 ppt ASW (as required), and counted using a hemacytometer (Corning).

### Denaturing Gradient Gel Electrophoresis (DGGE).

DGGE was used to determine the composition of the bacterial species during add-back experiments. PCR amplification of 16SrDNA was done according to the method of Ferris et al., 1996, and the products analyzed by DGGE according to Wang et al (2005). The oligonucleotide primers used to amplify the 16S rDNA gene specific for most eubacteria were 1055f (ATGGCTGTCGTCAGCT) (SEQ ID NO. 15) and 1392rGC (CGCCCGCCGCGCCCCGCGCCCGGCCC-GCCGCCCCCGCCCCACGGGCGGTGTGTAC) (SEQ ID NO. 16) containing a GC-clamp.

### Results

Visualization and localization of Silicibacter sp. TM1040 on dinoflagellate cells.

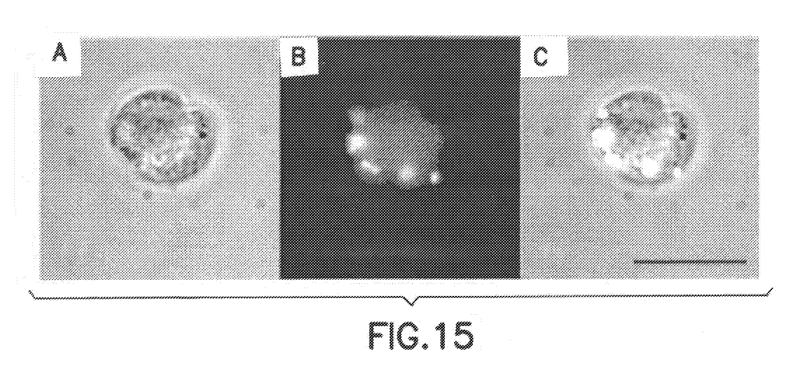

The interaction of Silicibacter sp. TM1040 with P. piscicida was measured by microscopic imaging of live motile bacteria that had been stained with a fluorescent dye (CFDA/SE). Fluorescent and phase-contrast images of the identical microscope field containing dinoflagellate zoospores and bacteria were superimposed and analyzed by computer image analysis software to localize the bacteria on the dinoflagellate. FIG. 15 shows a representative set of images from this analysis. A single zoospore is shown in FIG. 15A, and several labeled bacteria are visible in panel B. When the two images are superimposed the location of the bacteria relative to the dinoflagellate cell is revealed (FIG. 15C). As shown in FIG. 15C, this analysis indicates that the interaction of Silicibacter sp. TM1040 with P. piscicida results in the bacteria being in close physical association with the dinoflagellate. Bacteria were often observed on the dinoflagellate periphery, but they were also found at sites either within or on top of the zoospore, although it was not possible to distinguish between these two possibilities by this method. These data show that Silicibacter sp. TM1040 forms a close physical interaction with P. piscicida, where they appears to attach to the surface of the dinoflagellate, and shows that the bacteria may also find their way to locations beneath the surface of the dinoflagellate cell or in deep grooves on the outer surface of the host cell.

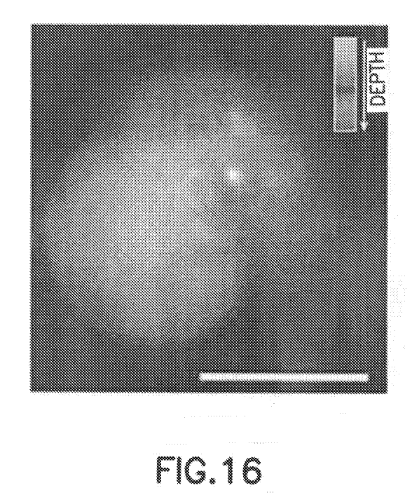

To obtain a more precise location of Silicibacter sp. TM1040 cells when they interact with the dinoflagellate, a series of Z-section optical images using confocal scanning laser microscopy (CSLM) were obtained. The optical sections were combined into a composite image in which the objects were color-coded based on their Z-axis distance (depth). FIG. 16 shows a representative image from this analysis. In these images, green pixels represent objects that are close to the proximal side of the dinoflagellate cell surface, blue tints denote objects on the distal side of the dinoflagellate, and a pink or red color is used for objects in the center of the zoospore. Bacteria were found on the periphery of the dinoflagellate (as observed in FIG. 15), but were also frequently observed at sites that co-localize to areas beneath the surface of the dinoflagellate. For example, in FIG. 16, at least five bacteria are visible in the Z-axis center of the epitheca (or upper hemisphere) of the dinoflagellate cell, and one bacterium is seen beneath the surface of the hypotheca or lower hemisphere of the P. piscicida zoospore. Results of a computer image analysis of these images indicate that these cells co-localize within optical sections scanned between 5-10 μm below the dinoflagellate surface, and support the hypothesis that motile Silicibacter sp. TM1040 cells form an intimate association with P. piscicida.

Random mutagenesis of Silicibacter sp. TM1040.

Silicibacter sp. TM1040 is chemotactic toward P. piscicida cells as described above, indicating that bacterial motility is important for the initiation or formation of its interaction with P. piscicida. To verify this indication mutants of Silicibacter sp. TM1040 were constructed that were defective in wild-type motility. A technique was developed that permitted random mutations to be constructed using a Tn5 derivative (the EZ::TN <R6Kãori/KAN-2> transposome). The method is highly effective and has a mutation efficiency of ca. 1.5×10⁻⁴ kanamycin-resistant colonies per electroporation (data not shown). Using this method of transposon insertional mutagenesis, a bank of 3,724 kanamycin-resistant Silicibacter sp. TM1040 mutants was constructed. The mutants were screened for loss of motility and auxotrophy. Twenty eight non-motile (Mot−) mutants, i.e., cells that had non-wild-type swimming, including non-motile cells as well as cells that were motile, albeit with extreme impairment of flagellar function, were identified. By comparison, 6 independent auxotrophs were obtained from the bank. Further tests reduced the number of (Mot−) mutants to nine, of which three, TM2014, TM2017, and TM2038, were chosen for further analysis.

Phenotypic Analysis of Silicibacter sp. TM1040 Motility Mutants.

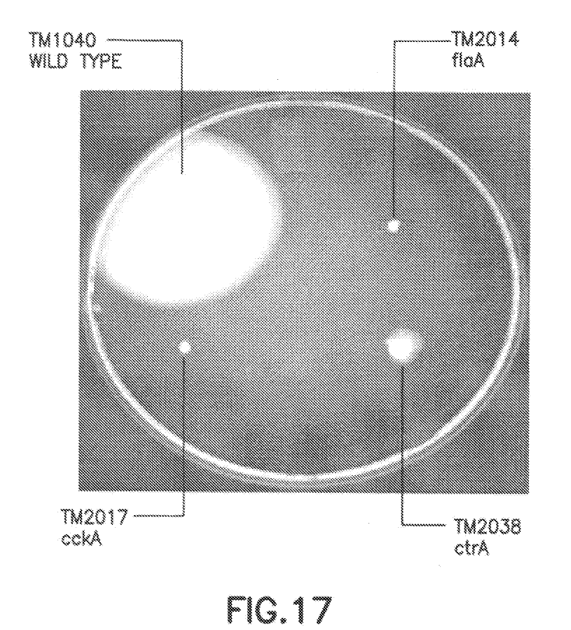

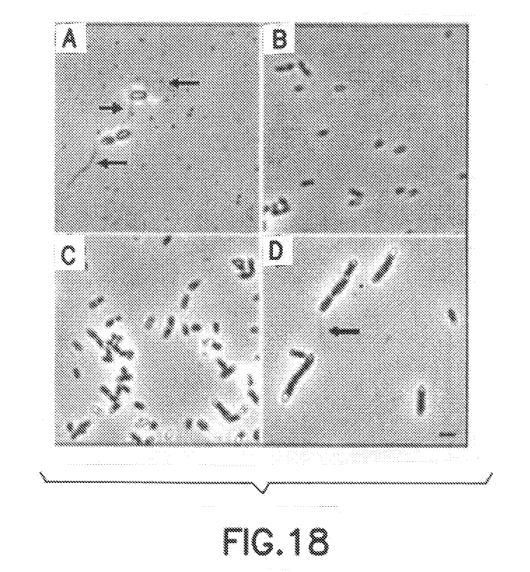

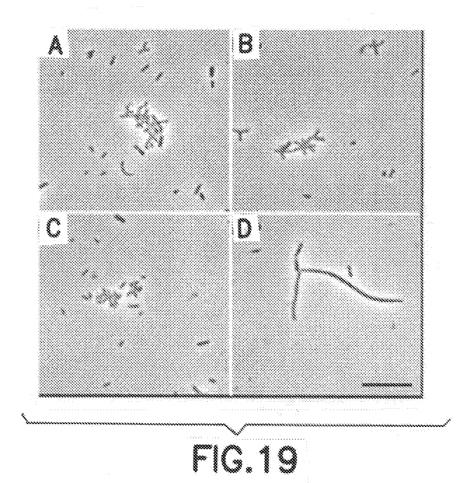

Two of the three strains, TM2014 and TM2017, are non-motile (Mot−) in marine motility agar (FIG. 17) and do not swim in liquid media. Neither of the two mutants produces flagella, as measured by a silver staining method (FIG. 18). Both are capable of forming rosettes, star-shaped clusters of cells typical of this species, and their growth rates and cell size are indistinguishable from wild-type (FIG. 19 and Table 7). Conversely, strain TM2038 is non-motile when examined within 24-48 h post inoculation, but produces small flares of motile cells in semi-solid marine motility agar upon prolonged incubation (FIG. 17). In liquid media, the majority (>99.9%) of the cells were nonmotile.

Silver staining of flagella confirmed that a few TM2038 cells produced flagella (FIG. 18). These data indicate that the mutation in TM2038 leads to a severe down-regulation of flagellar synthesis. Interestingly, the mutation also resulted in an increase in the length of TM2038 cells (FIG. 19). The mean length of a TM2038 cell is 6.9 μm or nearly four times the length (1.5 μm) of the parent cell, Silicibacter sp. TM1040 (Table 7). Taken together, these results suggest that the mutation in TM2038 has likely affected a regulatory circuit that controls flagellar synthesis and cell elongation.

### Genomic Analysis of the Mutations Leading to Motility Defects.

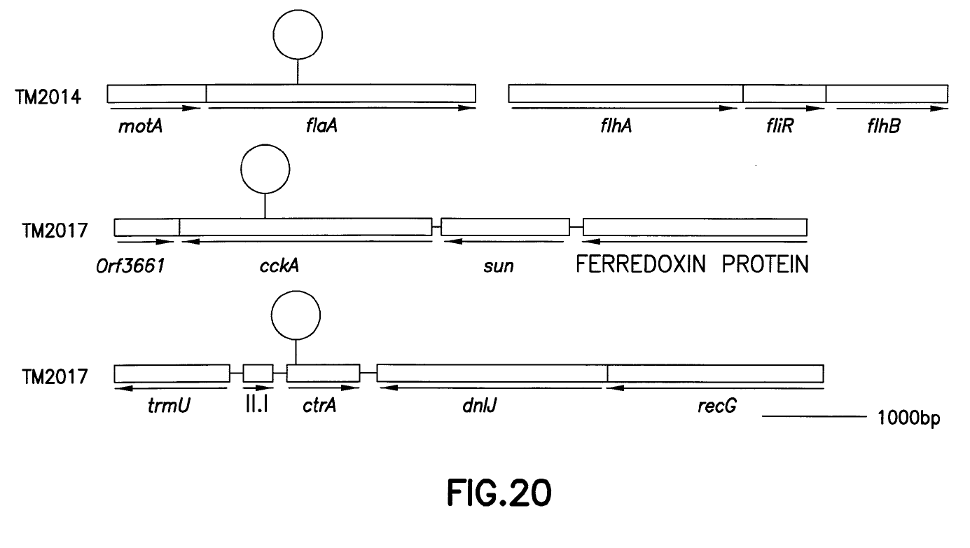

The transposon and flanking DNA sequences from strains TM2014, TM2017, and TM2038 were obtained by rescue cloning as described above. Following trimming of transposon nucleotide sequence, the DNA flanking the site of each insertion was used to search for DNA:DNA homology to the draft annotation of the genome of Silicibacter sp. TM1040 (http://genome.ornl.gov/microbial/rose_tm1040). Open reading frames (ORFs) were identified and the deduced amino acid sequence of these proteins was used in further searches with BLAST of sequences contained in GenBank and the draft annotation of another roseobacter species, Jannaschia sp. CCS1 (http://genome.ornl.gov/microbial/jann/). These data were used to establish the degree of homology between the mutated ORFs and known proteins in the databases. The results of this analysis are shown in FIG. 20. The Mot− mutant TM2014 has a mutation in a gene (GenBank accession number ZP_{—}003388108.1; JGI contig 52, ORF1857, and gene ID #402609350) that hereafter will be referred to as flaA. The flaA ORF has negligible homology to protein sequences from non-roseobacter genera, but reasonably good homology to ORFs of unknown function from both Silicibacter pomeroyi DSS-3 (GenBank accession number AAV93530.1; E=3.0 e-76 and 27% identities) and Jannaschia sp. CCS1 (JGI contig 27, ORF2948; E=8e-25 and 24% identities). This suggests that flaA may have a roseobacter specific function.

While flaA protein:protein searches failed to suggest a function for this ORF, an analysis of the ORFs adjacent to flaA was enlightening. flaA is the last gene in a group of five genes each aligned in the same transcriptional orientation, suggesting that flaA may be part of an operon. Two of the five genes in this operon have homology to genes encoding proteins known to be involved in flagellar biosynthesis or energetics. For example, flaA is downstream of the predicted stop codon for a gene encoding a protein that is homologous to Caulobacter crescentus motA (E=2.0 e-87, 55% identities). In addition, downstream of flaA and separate from it by a 335 bp gap is the start codon of a gene with nearly perfect homology to flhA from Rhodobacter sphaeroides (E=0.0, 65% identities) whose function is involved in flagellar protein export. Homologs to fliR and flhB, other genes encoding proteins required for flagellar export, are located downstream to the flhA homolog; further strengthening the belief that flaA encodes a protein involved in flagellar biosynthesis or export. The mutation in strain TM2017 (FIG. 20), the second Mot− mutant, is in an ORF (GenBank accession ZP_{—}00339586.1; JGI contig 56, ORF3662; gene ID #402627660) that has homology to many sensor histidine kinases, with significant homology to R. capsulatus CckA (E=1e-162, 58% identities). The Silicibacter sp. TM1040 CckA homolog is the last gene in a group of four ORFs each transcribed in the same direction (right to left, as shown in FIG. 20). The other genes in this locus encode proteins with homology to R. sphaeroides Fmu (Sun family of 16S rRNA cytosine methyltransferase), R. sphaeroides ferrodoxin, and L-carnitine dehydratase from Cupriavidus necator (formerly Ralstonia eutropha JMP134).

The mutation that produced strain TM2038 is in an ORF (GenBank accession ZP_{—}00339421.1; JGI contig 56, ORF 3516; gene id 402626200) with homology to the response regulator protein, CtrA (FIG. 6) of R. capsulatus (E=1.0 e-116, 89% identity). CtrA, a DNA binding protein, acts in concert with CckA as a two-component regulatory circuit (Hoch et al., 1995) to regulate motility and genetic exchange in R. capsulatus (Lang et al., 2002) and the cell cycle of C. crescentus (Jacobs et al., 2003), perhaps offering clues as to the homologous gene functions in Silicibacter sp. TM1040.

### Defects in Bacterial Motility Adversely Affect the Physical Interaction.

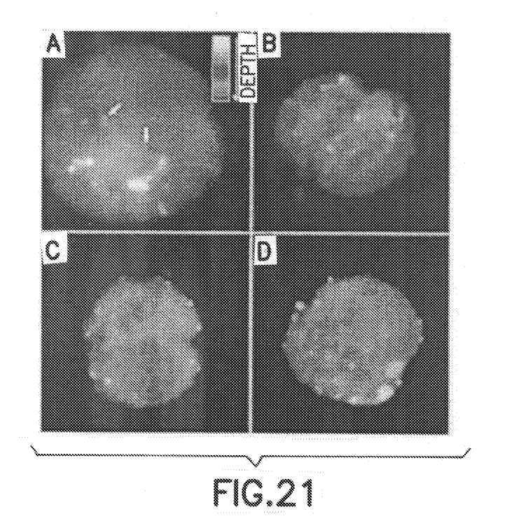

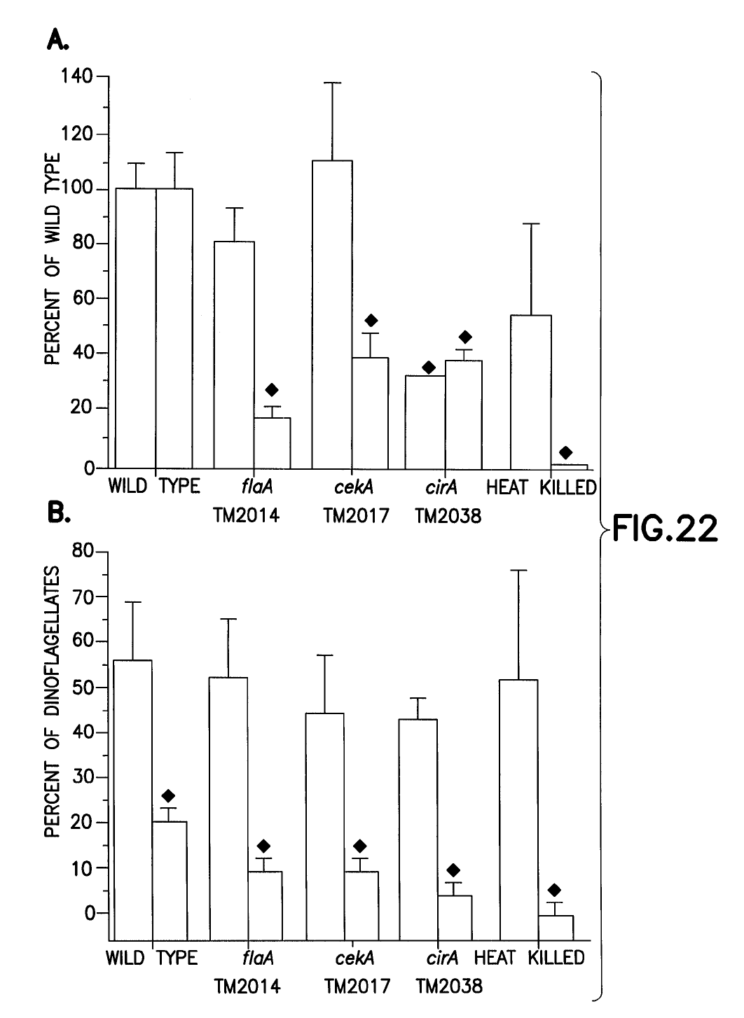

The Mot-mutants were compared to the wild-type parent for their ability to physically interact with P. piscicida zoospores by analyzing CSLM composite images of fluorescently-tagged bacteria. FIG. 21 shows a representative set of these composite images. When compared to the wild-type bacteria (FIG. 21A), micrographs of the three Mot− strains (FIGS. 21B-D) showed fewer bacteria colocalized to areas beneath the zoospore surface (or interior portions of the dinoflagellates). For example, images of the flaA and ctrA mutant, FIGS. 21B and C respectively, lack fluorescently stained bacterial cells that co-localize with cytoplasmic regions of the zoospore (red or pink colors), yet the presence of bacteria on the periphery (green or blue color) of the dinoflagellate is readily apparent. The lack of bacteria that co-localized to regions beneath the surface or interior of the dinoflagellate was also evident with TM2038 (the ctrA mutant), which also appeared to have reduced ability to attach to the surface of the zoospore (FIG. 21D). A quantitative measurement of the number of bacterial cells either co-localized to the surface or interior regions of the zoospore (FIG. 22) supports the CSLM images. Individual dinoflagellates harbored between 1 to 6 attached wild-type bacteria per dinoflagellate and from 0 to 6 intracellular wild-type bacteria per dinoflagellate. The mean of these values was set at 100% and used to compare the effect of the three mutations on the physical interaction with P. piscicida. The results, shown in FIG. 22, are consistent with the CSLM data: the Mot− strains are defective in their physical interaction with the zoospore. Both the flaA and cckA mutations have little effect on the attachment of these bacteria to the surface of the dinoflagellates (79.75% and 111.39% of wild type values, respectively), while the number of bacteria co-localized to the interior of the dinoflagellate was significantly reduced (16.13% and 38.71% of the wild-type value, respectively) in these two mutants. The ctrA mutant (TM2038) failed to localize to either the surface (31.65% of wild type) or the interior (38.71% of wild type) of the zoospore at the same level as the wild-type cells. The attachment of heat-killed Silicibacter sp. TM1040 cells was reduced to 54.43%, but this value was not statistically different from the wild-type.

However, no heat-killed bacteria were found to co-localize with intracellular regions of the dinoflagellate. This suggests that attachment to the dinoflagellate surface is not dependent on a living cell, while attachment to structures beneath the zoospore surface requires a living cell with functioning flagella.

Not all zoospores harbor bacteria, therefore, the percent of dinoflagellates with bacteria either localized to the surface or interior of the zoospore was measured (FIG. 22B). The percentage of zoospores in the population that harbored surface-co-localized bacteria when incubated with wild-type Silicibacter sp. TM1040 was 56%, while 21% of the dinoflagellates were observed to have bacteria that co-localized to areas beneath the surface of the zoospore (FIG. 22B, first pair of bars). Neither the flaA, cckA or ctrA mutation affected the attachment of the mutant cells to the dinoflagellate surface, however these mutations had a statistically significant negative effect on finding the bacteria co-localized with the interior of the zoospore (FIG. 22B). Taken as a whole, these results underscore the requirement of bacterial motility in the interaction of Silicibacter sp. TM1040 with P. piscicida, and suggest that roseobacter motility may be important in allowing the bacteria to gain access to areas that appear to be beneath the surface of the zoospore.

Non-Motile Bacteria Adversely Affect the Growth of P. piscicida.

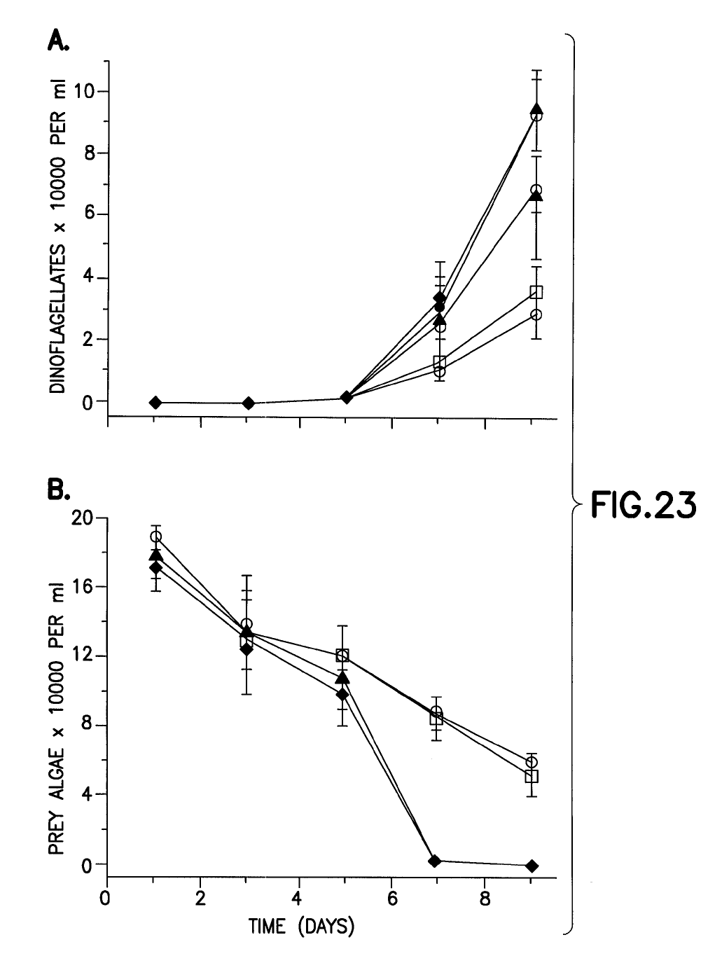

It is likely that bacterial motility may be important in the growth of that P. piscicida because of the interaction with a roseobacter, and this was tested using the mutant Silicibacter sp. TM1040 strains and axenic zoospores. Axenic P. piscicida zoospores were individually cultured in the presence of Silicibacter sp. TM1040 and each of the three Mot− mutants, added to a final concentration of 100 bacteria per ml of culture. As shown in FIG. 23A, in the presence of wild-type TM1040, dinoflagellate density reached 9.5×10³ cells per ml on day nine, compared to a zoospore density of 2.9×10³ cells per ml on day nine in the absence of bacteria. Dinoflagellate growth was reduced by ca. 30% when incubated with non-motile strains TM2014 (flaA) and TM2017 (cckA), but not affected by the presence of TM2038 (ctrA). With the increase in dinoflagellate cell density there was a concomitant reduction in Rhodomonas sp. prey algal cell density (FIG. 23B). In the presence of wild-type and the three mutant strains, prey algal densities decreased at a similar rate from ca. 1.7×10⁵ per ml on day one to ca. 9×10⁴ cells per ml on day five, followed by a dramatic decrease resulting in ca. 1.5×10³ cells per ml by day seven. Without the addition of bacteria or in the presence of heat-killed cells, prey algal densities decreased slowly from 1.7×10⁵ per ml on day one to 1.2×10⁵ cells per ml on day five and finally to 6×10⁴ per ml on day nine (FIG. 23B).

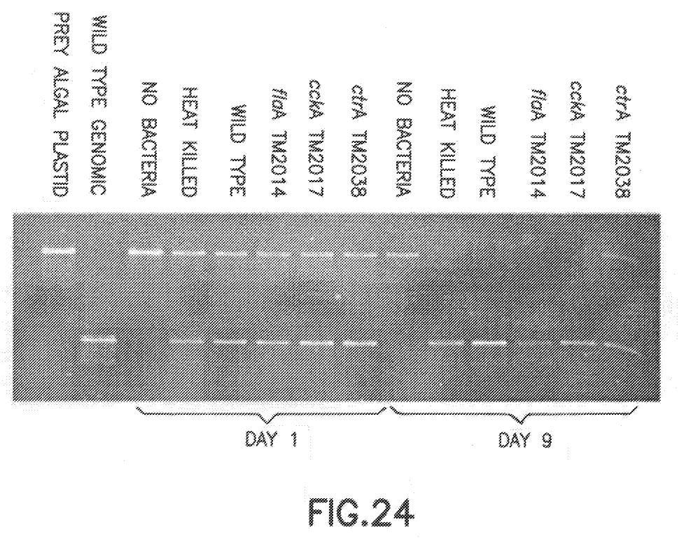

The bacterial species composition of the cultures was determined by DGGE (FIG. 24). The PCR primers used in this method amplify bacterial 16S rDNA as well as Rhodomonas sp. plastid DNA, serving as an internal positive control. A single DNA band corresponding to the Rhodomonas sp. plastid was present in the axenic P. piscicida culture on day one, indicating the lack of bacterial cells. Two DNA bands are found in all other cultures containing Silicibacter sp. TM1040 or mutant cells (FIG. 24, lower DNA band), Rhodomonas sp. algae (FIG. 24, upper DNA band), plus P. piscicida zoospores. The lack of additional 16S rDNA bands indicates that the cultures were populated solely by Silicibacter sp. TM1040 or one of the Mot− mutants. The loss of Rhodomonas sp. algae through consumption by the dinoflagellates can be observed in the samples from day nine. Together these data support the hypothesis that motility of Silicibacter sp. TM1040 plays a role in the physiological effects the bacteria have on promoting growth of P. piscicida.

A central component of this study was the construction of a library of Silicibacter sp. TM1040 random transposon insertion mutants that was subsequently screened to find those that exhibited defects in swimming motility. The successful use of transposon mutagenesis in Silicibacter sp. TM1040 represents a method that may be of value for the genetic manipulation of other Roseobacter clade species. The mutated gene in three of the Mot− strains was identified. All three mutations reveal interesting features of the molecular mechanism underlying flagellum biosynthesis and energetics in Silicibacter sp. TM1040. The data indicate that Silicibacter sp. TM1040 swimming motility is in part controlled by homologs of a two-component regulatory circuit including a sensor kinase, CckA, and response regulator, CtrA. Silicibacter sp. TM1040 CckA and CtrA proteins are nearly identical in amino acid sequence and conserved domains to the same proteins in other α-Proteobacteria, such as R. capsulatus, C. crescentus, and Sinorhizobium meliloti. There is ample evidence that CckA/CtrA regulate a variety of cellular functions, including motility, cell differentiation, and genetic exchange. For example, in R. capsulatus, CckA/CtrA regulate transcription of class II, class III, and class IV flagellar genes (Lang et al., 2002). The ctrA mutation resulted in cells that fail to divide normally, giving rise to an elongated cell phenotype that is very similar to the phenotype observed in C. crescentus ctrA strains (Reisenauer et al., 1999).

The third mutant, strain TM2014, harbors a transposon insertion in a novel flagellar gene, flaA, whose deduced amino acid sequence shares little homology with other non-roseobacter proteins.

Disruption of flaA results in a complete loss of motility and flagellar biosynthesis. Based on the phenotype and the proximity of flaA to ORFs homologous to flagellar type III secretion system export proteins (Macnab, R. M. 2004), FlaA may play a role in the biosynthesis of the flagellum, perhaps in the Silicibacter sp. TM1040 flagellar TTSS pathway.

Silicibacter sp. TM1040 interacts with P. piscicida through a mutualistic or symbiotic relationship, where both organisms benefit from the interaction. For the bacteria one significant benefit is the acquisition of nutrients from the dinoflagellate in the form of DMSP and amino acids. The bacteria use swimming motility and chemotaxis to move towards concentration gradients of nutrients produced by the swimming dinoflagellate. As the bacteria close in on their host, they are confronted with several options, including to swim along with the moving dinoflagellate, to find dinoflagellates that have settled and orbit around these non-motile hosts, or to come in close proximity to the zoospore whereupon the bacteria attach. The latter appears most feasible based on the data set forth herein. In this niche, the bacteria not only are bathe in the nutrients they require, but also may be in a safe-zone, free from ciliates and flagellate bacteriovores.

The benefit to the dinoflagellate is not nearly so obvious, although the data show that zoospore growth is improved by the presence of Silicibacter sp. TM1040. Bacteria may enhance the growth of algal species by several different mechanisms. In dinoflagellate species, bacteria are thought to remove the build up of toxic waste products, regenerate essential nutrients, or produce compounds necessary for dinoflagellate growth (Doucette, 1995). In the case of P. piscicida, whole algal cells are supplied as the food source. The consumption and digestion of these algal cells could result in the build up of excess carbon or other compounds in the culture that limit dinoflagellate growth. The bacteria may function to degrade those compounds as they accumulate. Silicibacter sp. TM1040 may be required by P. piscicida to achieve balanced growth. This is the basis for a number of symbiotic and/or syntrophic relationships among organisms in nature. In another α-Proteobacterium genus, Rhizobium, nitrogen is supplied to the plant host in return for carbon (Ausubel, 1982).

As noted hereinabove, Silicibacter sp. TM1040 is a culturable, genetically tractable member of the marine Roseobacter clade, originally isolated from a culture of the dinoflagellate Pfiesteria piscicida, which forms an intimate and obligate interaction with algae, itself originally obtained from the Chesapeake Bay in 1997 (1, 17). The bacterium is associated with, attaches to, and is physiologically required by its dinoflagellate host (2, 16-18). TM1040 is found associated with other dinoflagellates and algal cells in laboratory cultures, and is frequently found in water samples obtained from the Chesapeake Bay.

The results of several experiments as set forth herein show that extracts and/or cell-free filtrates obtained from culture supernatants of TM1040 have a potent antibacterial activity that is effective in killing several pathogenic bacteria.

The cultivation process comprises culturing Silicibacter sp. TM1040 under aerobic conditions, in either a static or shaking mode, in a nutrient medium containing one or more sources of carbon, nitrogen and optionally nutrient inorganic salts and/or trace elements, followed by isolation of the said compound and purification in a customary manner.

The nutrient medium preferably contains sources of carbon, nitrogen and nutrient inorganic salts, organic trace elements and optionally other trace elements. The carbon sources are, for example, starch, glucose, sucrose, dextrin, fructose, molasses, glycerol, lactose or galactose, preferably glucose. Amount of the carbon source added varies according to the kind of the carbon source, and usually 1 to 100 g, preferably 2 to 50 g per 1 liter medium.

The sources of nitrogen are, for example, soybean meal, peanut meal, yeast extract, beef extract, peptone, tryptone, malt extract, corn steep liquor, gelatin or casamino acids, preferably soybean meal and corn steep liquor. Amount of the nitrogen source added varies according to the kind of the nitrogen source, and usually 0.1 to 30 g, and preferably 1 to 10 g per 1 liter medium

As the organic trace nutrients, amino acids, vitamins, fatty acids, nucleic acids, those containing these substances such as peptone, casamino acid, yeast extract and soybean protein decomposition products are used. Amount of the special required substance used varies according to the kind of the substance, and usually ranges between 0.2 g to 200 g, and preferably 3 to 100 g per 1 liter medium.

The nutrient inorganic salts and trace elements are, for example, sodium hydrogen phosphate, potassium hydrogen phosphate, sodium chloride, cobalt chloride, calcium chloride, calcium carbonate, potassium nitrate, ammonium sulfate or magnesium sulfate, preferably cobalt chloride and calcium carbonate. Amount of inorganic acid varies according to the kind of the inorganic salt, and usually 0.001 to 10 g per 1 liter medium.

Cultivation of the culture is usually carried out at temperatures between 20-42° C. and in a medium having a pH from about 6.0 to about 8.0, and preferably, about 7.0 to about 8.0. Preferably, the medium is maintained at a pH and salinity value appropriate for growth of the Silicibacter sp. TM1040 bacteria, for about 50 to about 200 hours, under aerobic condition provided by shaking or aeration/agitation in order to obtain an optimal yield of the bacteria and antibacterial agent.

Finally, the antibacterial agent may be isolated and purified from the culture. Namely, microbial cells are separated from the culture by a conventional means such as centrifugation or filtration, and the cells or the medium are subjected to an extraction with a suitable solvent. As a solvent for the extraction, any substance in which the isolated agent is soluble can be used. For example, organic solvents such as acetone, chloroform, dichloromethane, hexane, cyclohexane, methanol, ethanol, isopropanol, benzene, carbon disulfide, diethyl ether etc., are used, and preferably chloroform, dichloromethane, acetone, methanol, ethanol or isopropanol is used. The purification can be carried out by conventional procedures such as absorption, elution, dissolving and the like, alone or preferably in combination.

The crude material can be further purified by using any of the following techniques: normal phase chromatography using alumina or silica gel as stationary phase and eluants such as ethyl acetate, chloroform, methanol or combinations thereof; reverse phase chromatography using reverse phase silica gel like dimethyloctadecylsilylsilica gel, also called RP-18, or dimethyloctylsilylsilica gel, also called RP-8; as stationary phase and eluants such as water, buffers such as phosphate, acetate, citrate (pH 2-8), and organic solvents such as methanol, acetonitrile, acetone, tetrahydrofuran or combinations of these solvents; gel permeation chromatography using resins such as SEPHADEX™ LH-20 (Pharmacia Chemical Industries, Sweden), TSKgel TOYOPEARL™ HW-40F (TosoHaas, Tosoh Corporation, Japan) in solvents such as methanol, chloroform, acetone, ethyl acetate or combinations of these solvents or SEPHADEX™ G-0 and G-25 in water; or counter-current chromatography using a biphasic eluant system made up of two or more solvents such as water, methanol, ethanol, isopropanol, n-propanol, tetrahydrofuran, acetone, acetonitrile, methylene chloride, chloroform, ethyl acetate, petroleum ether, benzene and toluene.

It is contemplated that the present invention may be practiced using either batch, fed-batch or continuous processes and that any known mode of fermentation would be suitable. A classical batch fermentation is a closed system where the composition of the medium is set at the beginning of the fermentation and not subjected to artificial alterations during the fermentation. Thus, at the beginning of the fermentation, the medium is inoculated with the desired organism or organisms and fermentation is permitted to occur adding nothing to the system. Typically, however, a batch fermentation is “batch” with respect to the addition of carbon source and attempts are often made at controlling factors such as pH and oxygen concentration. In batch systems the metabolite and biomass compositions of the system change constantly up to the time the fermentation is stopped. Within batch cultures, cells moderate through a static lag phase to a high growth log phase and finally to a stationary phase where growth rate is diminished or halted. If untreated, cells in the stationary phase will eventually die.

A variation on the standard batch system is the fed-batch system. Fed-batch fermentation processes are also suitable in the present invention and comprise a typical batch system with the exception that the substrate is added in increments as the fermentation progresses. Fed-batch systems are useful when catabolite repression is apt to inhibit the metabolism of the cells and where it is desirable to have limited amounts of substrate in the medium. Using a fed-batch system, it is possible to maintain a steady concentration of substrate while accommodating maximum bioconversion of the substrate to product.

Batch and fed-batch fermentations are common and well known in the art and examples may be found in, for example Brock, Thomas D., In Biotechnology: A Textbook of Industrial Microbiology, 2nd ed.; Sinauer Associates, Inc.: Sunderland, Mass., 1989.

Continuous fermentation is an open system wherein a defined fermentation medium is added continuously to a bioreactor and an equal amount of conditioned medium is removed simultaneously for processing. Continuous fermentation generally maintains the cultures at a constant high density. Continuous fermentation allows for the modulation of one factor or any number of factors that affect cell growth or end product concentration. For example, one method will maintain a limiting nutrient such as the carbon source or nitrogen source at low concentration and allow all other parameters to be in excess. In other systems, a number of factors affecting growth can be altered continuously while the cell concentration, measured by medium turbidity, is kept constant. Continuous systems strive to maintain steady state growth conditions and thus the cell loss due to medium being drawn off must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, supra.

It is a further objective of this invention to provide an inexpensive means for controlling potentially harmful microorganisms that may be present, for example, in aqueous-containing systems, such as aquariums. The methods are also applicable to the sterilization of effluents from sewage treatment facilities, including sludge, and contaminated effluents from manufacturing plants.

It is an additional object of this invention to provide a method for protecting the surfaces of various articles against the growth of microorganisms by incorporating an antibacterial agent of the present invention into coatings for applying on said surfaces. Thus, the present invention provides for an antifouling composition comprising a carrier suitable for underwater application and an effective antifouling amount of at least one bioactive agent derived from TM1040 strain of the present invention. Thus, the present invention is directed to antibacterial compounds and/or extracts of TM1040, an aquatic microorganism that has the ability to repel, prevent or otherwise deter aquatic pests from settling on or near target locations.

In one embodiment, TM1040 bacteria may be removed from the culture medium and then blender homogenized in distilled water (about 1:3 weight/volume). The resulting homogenate can be lyophilized, resuspended in hexane and sonicated for about 30 minutes. The resulting mixture can then be centrifuged resulting in a supernatant and a pellet. The solvent can then be removed from the supernatant using a vortex vacuum evaporator. The pellet can be extracted, centrifuged and the resulting supernatant is separated from the resulting pellet. Each extract can easily be tested for antibacterial activity as described below.

In an alternative embodiment, the collected, cleansed TM1040 microorganism can be blended and extracted overnight by immersion in solvent. After extraction, the liquid is removed and centrifuged and the supernatant is vacuum dried to obtain a solvent extract concentrate. Tissues remaining in the beaker are air dried, e.g., in a hood, and subjected to further solvent extraction and each extract is then tested for antibacterial activity as described below.

Assays may be conducted by assaying for bacterial inhibition activity, mussel byssal attachment activity, bacterial anti-settlement activity, and larvae anti-settlement activity.

To perform the bacterial inhibition assay a dried extract is dissolved in about 2 ml of original solvent to get a saturated solution. About twenty to fifty (20-50) ul of each solution is added to a sterile bio-assay disc (6 mm Difco™ 1599-33) and air dried. Three discs with extract and two control discs with only solvent (all vacuum dried) are placed on a semi-solid (half usual concentration) tryptic soy agar (TSA) plate inoculated with a dilute microbial suspension. The plates are incubated for about 24 hours at room temperature. Different bacteria can be used in the antibiotic assay for each extract. The halo around the disc is measured and the assay scored depending on the size of the halo.

To perform the mussel byssal thread attachment assay, juvenile blue mussels, Mytilus edulis, are used. The sample zone is coated with TM1040. The mussels are fixed around the edge of the sample zone using a commercial underwater compatible glue. The bioassay slides prepared in this way are placed at the bottom of an aquarium with running sea water. The mussels will attempt to attach themselves on the slide using many byssal threads. If an extract was antibacterial or active as a biorepellent, most byssal threads secreted by mussels avoid the sample zone.

To perform the bacterial anti-settlement assay, an extract is spread onto the frosted portion of glass slides and the solvent evaporated. An equal number of control slides should be prepared. Each slide is placed for contact with bacteria that is considered to be detrimental. The growth of the attached bacteria is counted.

Thus, in another aspect, the present invention provides compositions that reduce or completely eliminate fouling of underwater structures by aquatic pests. The compositions include a carrier, which contains at least one of the above-described antibacterial or antifouling agents that repel, prevent or otherwise deter aquatic pests from settling on structures incorporating the compositions. In accordance with one aspect of the present invention, the unique combination of carrier and antifouling agent augment one another by creating a slippery surface which causes problems for organisms attempting to anchor on the surface and, further, a chemically hostile local environment that the organisms find “distasteful” and in some cases toxic.

The antibacterial agent according to the present invention can be incorporated into structural members to provide aquatic pest repellent structures that are intended to be placed in aquatic environments. In this manner, the structure itself has integral aquatic pest repellency. The inventive antibacterial agents can also be incorporated into surface coatings of structures intended for underwater use. Materials that can incorporate the antifouling agents are known and must be compatible with the antibacterial/antifouling agents, i.e., there is no interaction between the materials and the bioactive agents that degrades or is otherwise detrimental to the activity of the agents, including phenolic resins, silicone polymers, chlorinated rubbers, coal tar and epoxy combinations, epoxy resin cured from a solvent solution with polyfunctional amines, polyamide resins, vinyl resins in solvent solutions, elastomers, fluoropolymers, polyesters and polyurethanes.

Vehicles (a structure or coating) which contain one or more repellent agents provide a medium, which allow the antibacterial agents to exert bioactivity in the locus to be protected over a period of time either by sustained release of the agent(s) or by creating a fixed effective surface concentration of the agent.

Diffusional systems are well suited to release the antibacterial agents to target areas. Diffusional systems include reservoir devices in which a core of the antibacterial agent is surrounded by a porous membrane or layer, or matrix devices in which the bioactive agent is distributed throughout an inert matrix.

Bioactive agents according to the present invention may be applied as surface coatings by painting or otherwise bonding or adhering a liquid or paste-like composition containing the repellent to the material intended for underwater use. The coatings may be applied in a variety of ways that are known in the art.

Microencapsulation techniques are useful in maintaining sustained focal release of bioactive agents according to the present invention. Microencapsulation may also be used for providing improved stability of the antifouling composition. The active agents of the present invention may be microencapsulated in structures in the form of spheres, aggregates of core material embedded in a continuum of wall material, capillary designs or incorporated into films and paints. The core material of a microcapsule containing a bioactive agent of the present invention may be in the form of a liquid droplet, an emulsion, a suspension of solids, a solid particle, or a crystal. The microcapsule coating material may be an organic polymer, hydrocolloid, wax, fat, lipid, metal, or inorganic oxide. Silicone polymers are the most preferred microcapsule coating material for use with the present invention.

The bioactive repellent in association with an acceptable carrier may be applied to submersible or submerged surfaces such as water intake systems, water cooling tubes, heat exchangers, and any other surfaces that are subject to biofouling. For example, the composition may be employed as an antifouling composition for boat hulls, fishing netting, buoys, pilings, lumber, roofs, and concrete. Dipping, spraying, brushing and laminating are other means for applying the antifouling composition.

Furthermore, the novel antifouling composition may be used for removing microorganisms from surfaces in hospitals or other surfaces where an aseptic environment is desirable.

In another embodiment, a nucleotide sequence encoding for the antibacterial agent is included in an expression vector or plasmid for transfection and expression in a compatible bacteria. For example, E. coli can be transformed using pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own polypeptides.

In addition to prokaryotes, eukaryotic microbes, such as yeast can also be used. Saccharomyces cerevisiae or common baker's yeast is the most commonly used among eukaryotic microorganisms, although a number of other strains are commonly available. For expression in Saccharomyces, the plasmid YRp7, for example, is commonly used. This plasmid already contains the trp1 gene, which provides a selection marker for a mutant strain of yeast lacking the ability to grow in tryptophan, for example ATCC No. 44076 or PEP4-1. The presence of the trp1 lesion as a characteristic of the yeast host cell genome then provides an effective environment for detecting transformation by growth in the absence of tryptophan. Suitable promoter sequences in yeast vectors include the promoters for 3-phosphoglycerate kinase or other glycolytic enzymes such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

In constructing suitable expression plasmids, the termination sequences associated with these genes are also introduced into the expression vector downstream from the sequences to be expressed to provide polyadenylation of the mRNA and termination. Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin or replication and termination sequences is suitable.

The examples and embodiments depicted in this specification are not intended to be limitations on the inventive concept described herein. Accordingly, one with skill in the art may make modifications in the methods and products that are intended to be covered by the following claims.

### Examples

### TM1040 Produces an Antibacterial Activity

Sterile filtrates and ethyl acetate extracts of spent TM1040 culture medium were placed into wells cored in a nutrient agar medium in which Vibrio anguillarum had been inoculated (FIG. 1). Following incubation at room temperature for 48 h, the plates were scored for the presence of a zone of clearing around each well, indicating that V. anguillarum had been killed or the growth of the bacteria significantly inhibited. Roseobacter 27-4 served as a positive control in this experiment and produced significant antibacterial activity when grown under static conditions in broth, but not when incubated with vigorous shaking. This result agrees with that reported by Bruhn et al (9). As can be observed in FIG. 1, a zone of clearing was also observed around the well containing the TM1040 extract.

Antibacterial Activity is Correlated with Pigment Production

A nonpigmented variant of TM1040 (FIG. 1, static, lower left well) produced less antibacterial activity than the wild-type strain (FIG. 1, shaking, lower left well), which agrees with the observations reported by Bruhn et al (9) for 27-4.

### Production of the Antibacterial Activity Occurs in Both Static and Shaking Broth Cultures.

TM1040 that was grown with vigorous shaking produced a much larger zone of inhibition than the control (Roseobacter 27-4; FIG. 1). This was unexpected, since previous reports (9) indicated that the antibacterial activity was only produced in under static growth conditions. This result was confirmed and the results shown in FIG. 2. Although the plate bioassay for antibacterial activity is only semiquantitative, TM1040 produces ca. 4-5× more activity when grown under shaking (aerobic) conditions than under static or anaerobic conditions. TM1040 produces comparable levels of antibacterial activity to 27-4 under static growth, but ca. 8-10× greater activity when the bacteria are grown aerobically (shaking culture). This suggests that the antibacterial activity of TM1040 is constitutively expressed, compared to that of 27-4, which is only expressed in broth media under static conditions.

The TM1040 antibacterial activity kills Mycobacterium marinum, Vibrio anguillarum, V. coralliilyticus, and V. shiloi.

Culture medium extracts from TM1040 grown under static conditions were used to test their effect on inhibiting the growth of known human, fish, and coral pathogens. M. marinum, a close relative of M. tuberculosis (the causative agent of human tuberculosis), is recognized as a pathogen of humans and many animals, including fish (3). In aquaculture, M. marinum is considered the primary causative agent of fish mycobacteriosis, although several Mycobacterium species associated with tubercle granulomas in aquarium, cultured, and wild fish populations have been described (20-22, 29). Mycobacteriosis in fish is characterized by emaciation, inflammation and ulceration of skin leading to open lesions, which reduce the overall condition of the fish, and often render the fish unsuitable for human consumption. The disease is especially prevalent in the Chesapeake Bay and in aquacultured striped bass underscoring the need to find suitable treatments to prevent the disease.

M. marinum was used in the plate bioassay to test the effectiveness of TM1040 culture extracts in killing this bacterium. The results shown in FIG. 3 confirm that TM1040 culture extracts possess one or more components capable of killing M. marinum.

Extracts of TM1040 were tested for their ability to inhibit the growth of V. coralliilyticus and V. shiloi, two pathogens of reef-building corals (4-7, 23). The results from these tests are shown in FIGS. 4 and 5. Extracts from both static and shaking cultures of TM1040 markedly inhibited the growth of these vibrios. Taken together with the earlier results, TM1040 extracts effectively kill three pathogenic Vibrio species: V. anguillarum, V. coralliilyticus, and V. shiloi.

### The Antibacterial Activity Causes No Adverse Reactions in Fish Larvae

Sterile filtrates of spent culture supernatants were tested for their effects on 2-3 day-old zebrafish (Danio rerio) larvae with the results set for below in Table 6.

TABLE 6
Zebrafish toxicity assay.

Dilution

| Supernatant Added | ¼ | ⅛ | 1/16 | 1/32 | 1/64 |
|---|---|---|---|---|---|
| TM1040 | 3/6^{a} | 5/6 | 6/6 | 6/6 | 6/6 |
| Uninoculated medium | 0/6 | 0/6 | 0/6 | 0/6 | 6/6 |
| Water | 6/6 | 6/6 | 6/6 | 6/6 | 6/6 |

^{a}Number of embryos alive after 24 h.

The viability of the fish embryos was unaffected by addition of TM1040 spent medium, except when at a very high concentration (1-to-4 dilution) where 3 of the 6 embryos died. Therefore, the antibacterial activity of TM1040 is not a general toxin and does not adversely affect larval fish. When dilutions of fresh uninoculated medium were added to the fish larvae, the embryos died even at the lowest concentration (1/64) used. These deaths were most likely caused by an increase in the autochthonous bacterial population naturally associated with the zebrafish. Addition of culture medium containing the TM1040 antibacterial activity in contrast had minimal affect on the health of the larvae, suggesting that the antibacterial activity in the filtrates prevented the death of the embryos.

### REFERENCES

The contents of all cited references are hereby incorporated by reference herein for all purposes.
1. Alavi, M., and R. Belas. 2001. Surface sensing, swarmer cell differentiation and biofilm development. Methods Enzymol. 336:29-40.
2. Alavi, M., T. Miller, K. Erlandson, R. Schneider, and R. Belas. 2001. Bacterial community associated with Pfiesteria-like dinoflagellate cultures. Environ. Microbiol. 3:380-396.
3. Belas, R., P. Faloon, and A. Hannaford. 1995. Potential applications of molecular biology to the study of fish mycobacteriosis. Annu. Rev. Fish Dis. 5: 133-173.
4. Ben-Haim, Y., E. Banim, A. Kushmaro, Y. Loya, and E. Rosenberg. 1999. Inhibition of photosynthesis and bleaching of zooxanthellae by the coral pathogen Vibrio shiloi. Environ. Microbiol. 1:223-229.
5. Ben-Haim, Y., and E. Rosenberg. 2002. A novel Vibrio sp. pathogen of the coral Pocillopora damicornis. Marine Biol. 141:47-55.
6. Ben-Haim, Y., F. L. Thompson, C. C. Thompson, M. C. Cnockaert, B. Hoste, J. Swings, and E. Rosenberg. 2003. Vibrio coralliilyticus sp. nov., a temperature-dependent pathogen of the coral Pocillopora damicornis. Int. J. Syst. Evol. Microbiol. 53:309-315.
7. Ben-Haim, Y., M. Zicherman-Keren, and E. Rosenberg. 2003. Temperature-regulated bleaching and lysis of the coral Pocillopora damicornis by the novel pathogen Vibrio coralliilyticus. Appl. Environ. Microbiol. 69:4236-4242.
8. Brinkhoff, T., G. Bach, T. Heidorn, L. Liang, A. Schlingloff, and M. Simon. 2004. Antibiotic production by a Roseobacter clade-affiliated species from the German Wadden Sea and its antagonistic effects on indigenous isolates. Appl. Environ. Microbiol. 70:2560-2565.
9. Bruhn, J. B., K. F. Nielsen, M. Hjelm, M. Hansen, J. Bresciani, S. Schulz, and L. Gram. 2005. Ecology, inhibitory activity and morphogenesis of a marine antagonistic bacteria belonging to the Roseobacter clade. Appl. Environ. Microbiol. in press.
10. Eilers, H., J. Pernthaler, J. Peplies, F. O. Glockner, G. Gerdts, and R. Amann. 2001. Isolation of novel pelagic bacteria from the German bight and their seasonal contributions to surface picoplankton. Appl. Environ. Microbiol. 67:5134-5142.
11. Gonzalez, J. M., R. Simo, R. Massana, J. S. Covert, E. O. Casamayor, C. Pedros-Alio, and M. A. Moran. 2000. Bacterial community structure associated with a dimethylsulfoniopropionate-producing North Atlantic algal bloom. Appl. Environ. Microbiol. 66:4237-4246.
12. Hjelm, M., O. Bergh, A. Riaza, J. Nielsen, J. Melchiorsen, S. Jensen, H. Duncan, P. Ahrens, H. Birkbech, and L. Gram. 2004. Selection and identification of autochthonous potential probiotic bacteria from turbot larvae (Scophthalmus maximus) rearing units. Syst. Appl. Microbiol. 27:360-371.
13. Hjelm, M., A. Riaza, F. Formoso, J. Melchiorsen, and L. Gram. 2004. Seasonal incidence of autochthonous antagonistic Roseobacter spp. and Vibrionaceae strains in a turbot larva (Scophthalmus maximus) rearing system. Appl. Environ. Microbiol. 70:7288-7294.
14. Jansen, M. 2000. Microbial demethylation of dimethylsulfoniopropionate and methylthiopropionate. Ph.D. Rijksuniversitet, Groningen, The Netherlands.
15. Liang, L. 2003. Investigation of secondary metabolites of North Sea bacteria: fermentation, isolation, structure elucitation and bioactivity. Dissertation. Universitat zu Gottingen, Gottingen, Sweden.
16. Miller, T. R., and R. Belas. 2004. Dimethylsulfoniopropionate (DMSP) metabolism by Pfiesteria-associated Roseobacter spp. Appl. Environ. Microbiol. 70:3383-3391.
17. Miller, T. R., and R. Belas. 2003. Pfiesteria piscicida, P. shumwayae, and other Pfiesteria-like dinoflagellates. Res. Microbiol. 154:85-90.
18. Miller, T. R., K. Hnilicka, A. Dziedzic, P. Desplats, and R. Belas. 2004. Chemotaxis of Silicibacter sp. TM1040 towards dinoflagellate products. Appl. Environ. Microbiol. 70:4692-4701.
19. Rao, D., J. S. Webb, and S. Kjelleberg. 2005. Competitive interactions in mixed-species biofilms containing the marine bacterium Pseudoalteromonas tunicata. Appl. Environ. Microbiol. 71:1729-1736.
20. Rhodes, M. W., H. Kator, I. Kaattari, D. Gauthier, W. Vogelbein, and C. A. Ottinger.
2004. Isolation and characterization of mycobacteria from striped bass Morone saxatalis from Chesapeake Bay. Dis. Aquat. Org. 61:41-51.
21. Rhodes, M. W., H. Kator, S. Kotob, P. van Berkum, I. Kaattari, W. Vogelbein, M. M. Floyd, W. R. Butler, F. D. Quinn, C. Ottinger, and E. Shotts. 2001. A unique Mycobacterium species isolated from an epizootic of striped bass (Morone saxatilis). Emerging Infect. Dis. 7:896-899.
22. Rhodes, M. W., H. Kator, S. Kotob, P. van Berkum, I. Kaattari, W. K. Vogelbein, F.
Quinn, M. M. Floyd, W. R. Butler, and C. A. Ottinger. 2003. Mycobacterium shottsii sp. nov., a slowly growing species isolated from Chesapeake Bay striped bass (Morone saxatilis). Int. J. Syst. Evol. Microbiol. 53:421-424.
23. Rosenberg, E., and Y. Ben-Haim. 2002. Microbial diseases of corals and global warming. Environ. Microbiol. 4:318-326.
24. Ruger, H. J., and M. G. Hofle. 1992. Marine star-shaped-aggregate-forming bacteria: Agrobacterium atlanticum sp. nov.; Agrobacterium meteori sp. nov.; Agrobacterium ferrugineum sp. nov., nom. rev.; Agrobacterium gelatinovorum sp. nov., nom. rev.; and Agrobacterium stellulatum sp. nov., nom. rev. Int. J. Syst. Bacteriol. 42:133-143.
25. Ruiz-Ponte, C., V. Cilia, C. Lambert, and J. Nicolas. 1998. Roseobacter gallaeciensis sp. nov., a new marine bacterium isolated from rearings and collectors of the scallop Pecten maximus. Int. J. Syst. Evol. Microbiol. 48:537-542.
26. Ruiz-Ponte, C., J. F. Samain, J. L. Sanchez, and J. L. Nicholas. 1999. The benefit of a Roseobacter species on the survival of scallop larvae. Marine Biotechnology 1:52-59.
27. Tsubotani, S., Y. Wada, K. Kamiya, H. Okazaki, and S. Harada. 1984. Structure of thiotropocin, a new sulfur-containing 7-membered antibiotic. Tetrahedron Lett. 25:419-422.
28. Uchino, Y., A. Hirata, A. Yokota, and J. Sugiyama. 1998. Reclassification of marine Agrobacterium species: proposals of Stappia stellulata gen. nov., comb. nov., Stappia aggregata sp. nov., nom. rev., Ruegeria atlantica gen. nov., comb. nov., Ruegeria gelatinovora comb. nov., Ruegeria algicola comb. nov., and Ahrensia kieliense gen. nov., sp. nov., nom. rev. J. Gen. Appl. Microbiol. 44:201-210.
29. Zeligman, I. 1972. Mycobacterium marinum granuloma. A disease acquired in the tributaries of Chesapeake Bay. Arch. Dermatol. 106:26-31.
30. Adler, J. 1966. Chemotaxis in bacteria. Science 153:708-716.
31. Altschul, S., W. Gish, W. Miller, E. Myers, and D. Lipman. 1990. Basic local alignment search tool. J. Mol. Biol. 215:403-410.
32. Ansede, J., P. J. Pellechia, and D. C. Yoch. 1999. Metabolism of acrylate to β-hydroxypropionate and its role in dimethylsulfoniopropionate lyase induction by a salt marsh sediment bacterium, Alcaligenes faecalis M3A. Appl. Environ. Microbiol. 65:5075-5081.
33. Ansede, J. H., P. J. Pellechia, and D. C. Yoch. 2001. Nuclear magnetic resonance analysis of [1-¹³C]dimethylsulfoniopropionate (DMSP) and [1-¹³C]acrylate metabolism by a DMSP lyase-producing marine isolate of the α-subclass of Proteobacteria. Appl. Environ. Microbiol. 67:3134-3139.
34. Ausubel, F. M. 2001. Current protocols in molecular biology. J. Wiley, New York, N.Y.
35. Ausubel, F. M. 1982. Molecular genetics of symbiotic nitrogen fixation. Cell 29:1-2.
36. Blackburn, N., T. Fenchel, and J. Mitchell. 1998. Microscale nutrient patches in planktonic habitats shown by chemotactic bacteria. Science 282:2254-2256.
37. Burkholder, J. M., E. J. Noga, C. H. Hobbs, H. B. Glasgow, Jr., and S. A. Smith. 1992. New ‘phantom’ dinoflagellate is the causative agent of major estuarine fish kills. Nature 358:407-410.
38. Cascales, R., and P. J. Christie. 2004. Definition of a bacterial type IV secretion pathway for a DNA substrate. Science 304:1170-1173.
39. Chambers, S. T., C. M. Kunin, D. Miller, and A. Hamada. 1987. Dimethylthetin can substitute for glycine betaine as an osmoprotectant molecule for Escherichia coli. J. Bacteriol. 169:4845-4847.
40. Christie, P. J. 2004. Type IV secretion: the Agrobacterium VirB/D4 and related conjugation systems. Biochim. Biophys. Acta 1694:219-234.
41. Christie, P. J., and J. P. Vogel. 2000. Bacterial type IV secretion: conjugation systems adapted to deliver effector molecules to host cells. Trends Microbiol. 8:354-360.
42. Cole, J. J. 1982. Interactions between bacteria and algae in aquatic ecosystems. Annu. Rev. Ecol. Syst. 13:291-314.
43. DeLoney-Marino, C. R., A. J. Wolfe, and K. L. Visick. 2003. Chemoattraction of Vibrio fischeri to serine, nucleosides, and N-acetylneuraminic acid, a component of squid light-organ mucus. Appl. Environ. Microbiol. 69:7527-7530.
44. Doucette, G. J. 1995. Interactions between bacteria and harmful algae: a review. Natural Toxins 3:65-74.
45. Ferris, M. J., G. Muyzer, and D. M. Ward. 1996. Denaturing gradient gel electrophoresis profiles of 16S rRNA-defined populations inhabiting a hot spring microbial mat community. Appl. Environ. Microbiol. 62:340-346.
46. Fuller, M. E., S. H. Streger, R. K. Rothmel, B. J. Mailloux, J. A. Hall, T. C. Onstott, J. K. Fredrickson, D. L. Balkwill, and M. F. DeFlaun. 2000. Development of a vital fluorescent staining method for monitoring bacterial transport in subsurface environments. Appl. Environ. Microbiol. 66:4486-4496.
47. Garg, B., R. C. Dogra, and P. K. Sharma. 1999. High-efficiency transformation of Rhizobium leguminosarum by electroporation. Appl. Environ. Microbiol. 65:2802-2804.
48. Giovannoni, S., and M. Rappe. 2000. Evolution, diversity, and molecular ecology of marine prokaryotes, p. 47-84. In D. L. Kirchman (ed.), Microbial ecology of the oceans. Wiley-Liss, New York, N.Y.
49. Gonzalez, J. M., and M. A. Moran. 1997. Numerical dominance of a group of marine bacteria in the alpha-subclass of the class Proteobacteria in coastal seawater. Appl. Environ. Microbiol. 63:4237-4242.
50. Gonzalez, J. M., R. P. Kiene, and M. A. Moran. 1999. Transformation of sulfur compounds by an abundant lineage of marine bacteria in the alpha-subclass of the class Proteobacteria. Appl. Environ. Microbiol. 65:3810-3819.
51. Gonzalez, J. M., J. S. Covert, W. B. Whitman, J. R. Henriksen, F. Mayer, B. Scharf, R. Schmitt, A. Buchan, J. A. Fuhrman, R. P. Kiene, and M. A. Moran. 2003. Silicibacter pomeroyi sp. nov. and Roseovarius nubinhibens sp. nov., DMSP demethylating bacteria from marine environments. Int. J. Syst. Evol. Microbiol. 53:1261-1269.
52. Gram, L., H. P. Grossart, A. Schlingloff, and T. Kiorboe. 2002. Possible quorum sensing in marine snow bacteria: production of acylated homoserine lactones by Roseobacter strains isolated from marine snow. Appl. Environ. Microbiol. 68:4111-4116.
53. Guillard, R. R. L. 1975. Culture of phytoplankton for feeding marine invertebrates. Plenum Press, New York, N.Y.
54. Hapfelmeier, S., N. Domke, P. C. Zambryski, and C. Baron. 2000. VirB6 is required for stabilization of VirB5 and VirB3 and formation of VirB7 homodimers in Agrobacterium tumefaciens. J. Bacteriol. 182:4505-4511.
55. Hoch, J., and T. Silhavy. 1995. Two-component signal transduction. American Society for Microbiology Press, Washington, D.C.
56. Hold, G. L., E. A. Smith, M. S. Rappe, E. W. Maas, E. R. B. Moore, C. Stroempl, J. R. Stephen, J. I. Prosser, T. H. Birkbeck, and S. Gallacher. 2001. Characterisation of bacterial communities associated with toxic and non-toxic dinoflagellates: Alexandrium spp. and Scrippsiella trochoidea. FEMS Microbiol. Ecol. 37: 161-173.
57. Jacobs, C., N. Ausmees, S. J. Cordwell, L. Shapiro, and M. T. Laub. 2003. Functions of the CckA histidine kinase in Caulobacter cell cycle control. Mol. Microbiol. 47:1279-1290.
58. Jansen, M., and T. A. Hansen. 1998. Tetrahydrofolate serves as a methyl acceptor in the demethylation of dimethylsulfoniopropionate in cell extracts of sulfate-reducing bacteria. Arch. Microbiol. 169:84-87.
59. Keller, M. D., and W. Korjeff-Bellows. 1996. Physiological aspects of the production of dimethylsulfoniopropionate (DMSP) by marine phytoplankton, p. 131-153. In R. P. Kiene, P. T. Visscher, M. D. Keller, and G. O. Kirst (ed.), Biological and environmental chemistry of DMSP and related sulfonium compounds. Plenum Press, New York, N.Y.
60. Kiene, R. P., L. J. Linn, J. Gonzalez, M. A. Moran, and J. A. Bruton. 1999. Dimethylsulfoniopropionate and methanethiol are important precursors of methionine and protein-sulfur in marine bacterioplankton. Appl. Environ. Microbiol. 65:4549-4558.
61. Kiene, R. P. 1996. Production of methanethiol from dimethylsulfoniopropionate in marine surface waters. Mar. Chem. 54:69-83.
62. Lang, A. S., and J. T. Beatty. 2002. A bacterial signal transduction system controls genetic exchange and motility. J. Bacteriol. 184:913-918.
63. Letunic, I., R. R. Copley, S. Schmidt, F. D. Ciccarelli, T. Doerks, J. Schultz, C. P. Lukashin, A. V., and M. Borodovsky. 1998. GeneMark.hmm: new solutions for gene finding. Nucleic Acids Res. 26:1107-1115.
64. Macnab, R. M. 2004. Type III flagellar protein export and flagellar assembly. Biochim. Biophys. Acta 1694:207-217.
65. Osing a, R., K. de Vries, W. Lewis, W. van Raaphorst, L. Dijkhuizen, and F. van Duyl. 1997. Aerobic degradation of phytoplankton debris dominated by Phaeocystis sp. in different physiological stages of growth. Aquat. Microb. Ecol. 12:11-19.
66. Ponting, and P. Bork. 2004. SMART 4.0: towards genomic data integration. Nucleic Acids Res. 32:142-144.
67. Reisenauer, A., K. Quon, and L. Shapiro. 1999. The CtrA response regulator mediates temporal control of gene expression during the Caulobacter cell cycle. J. Bacteriol. 181:2430-2439.
68. Sambrook, J., E. F. Fritsch, and T. Maniatis. 1989. Molecular cloning: a laboratory manual, 2nd ed. Cold Spring Harbor Laboratory Press, Cold Spring Harbor, N.Y.
69. Schmitt, R., I. Raska, and F. Mayer. 1974. Plain and complex flagella of Pseudomonas rhodos: analysis of fine structure and composition. J. Bacteriol. 117:844-857.
70. Shiba, T. 1991. Roseobacter litoralis gen. nov., sp. nov., and Roseobacter denitrificans sp. nov., aerobic pink-pigmented bacteria which contain bacteriochlorophylla. Syst. Appl. Microbiol. 14:140-145.
71. Visscher, P. T., and B. F. Taylor. 1994. Demethylation of dimethylsulfoniopropionate to 3-mercaptopropionate by an aerobic marine bacterium. Appl. Environ. Microbiol. 60:4617-4619.
72. Wang, Q., J. R. Deeds, A. R. Place, and R. Belas. 2005. Dinoflagellate community analysis of a fish kill using denaturing gradient gel electrophoresis. Harmful Algae 4:151-162.
73. Wei, X., and W. D. Bauer. 1998. Starvation-induced changes in motility, chemotaxis, and flagellation of Rhizobium meliloti. Appl. Environ. Microbiol. 64: 1708-1714.
74. West, M., N. M. Burdash, and F. Freimuth. 1977. Simplified silver-plating stain for flagella. J. Clin. Microbiol. 6:414-419.
75. Wolfe, A. J., and H. C. Berg. 1989. Migration of bacteria in semisolid agar. Proc. Natl. Acad. Sci. USA 86:6973-6977.
76. Yoch, D. C. 2002. Dimethylsulfoniopropionate: its sources, role in the marine food web, and biological degradation to dimethylsulfide. Appl. Environ. Microbiol. 68:5804-5815.
77. Yoch, D. C., J. H. Ansede, and K. S. Rabinowitz. 1997. Evidence for intracellular and extracellular dimethylsulfoniopropionate (DMSP) lyases and DMSP uptake sites in two species of marine bacteria. Appl. Environ. Microbiol. 63:3182-3188.
78. Zhong, Z., R. Caspi, D. Helinski, V. Knauf, S. Sykes, C. O'Byrne, T. P. Shea, J. E. Wilkinson, C. DeLoughery, and A. Toukdarian. 2003. Nucleotide sequence based characterizations of two cryptic plasmids from the marine bacterium Ruegeria isolate PR1b. Plasmid 49:233-252.
79. Zubkov, M. V., B. M. Fuchs, S. D. Archer, R. P. Kiene, R. Amann, and P. H. Burkill. 2001. Linking the composition of bacterioplankton to rapid turnover of dissolved dimethylsulphoniopropionate in an algal bloom in the North Sea Environ. Microb. 3:304-311.

1. An isolated Silicibacter sp. microorganism comprising a 16S ribosomal subunit nucleic acid sequence selected from the group consisting of:
(a) a nucleic acid sequence that has more than 95% identity to a nucleic acid sequence of SEQ ID NO 2; and
(b) a nucleic acid sequence fully complementary to a nucleic acid of (a).

2. A Silicibacter sp. delivery device, selected from among (i) a Silicibacter sp. delivery device for delivering a nucleotide sequence encoding a protein of choice for expression by an algae, the delivery device comprising a Silicibacter sp. microorganism comprising SEQ ID NO: 1, and (ii) a Silicibacter sp. delivery vector for delivery of genes from a bacterium to plant cells, the delivery vector comprising a Silicibacter sp. strain comprising: vir genes whose function is involved in the transfer of DNA from the bacterium to plant cells, wherein the Silicibacter sp. is TM1040 strain.

3. The isolated Silicibacter sp. microorganism of claim 1, comprising a 16S nucleotide sequence of SEQ ID NO: 2.

4. A protein of choice production system, comprising algae transfected with a recombinant Silicibacter sp. strain, wherein the Silicibacter sp. strain comprises a nucleotide sequence encoding for the protein of choice and at least one nucleotide sequence selected from the group consisting of SEQ ID NO. 3, SEQ ID NO. 4, SEQ ID NO. 5, SEQ ID NO. 6, SEQ ID NO.7, SEQ ID NO. 8, SEQ ID NO. 9, SEQ ID NO. 10, SEQ ID NO. 11, SEQ ID NO. 12, SEQ ID NO. 13, and SEQ ID NO. 14.

5. (canceled)

6. A method for delivering a nucleotide sequence encoding a protein of choice, the method comprising the steps of:
inserting nucleotide sequences encoding for the protein of choice into plasmid comprising at least one vir genes of Silicibacter sp. TM1040 strain of SEQ ID NO. 1;
infecting marine algae with the Silicibacter sp. strain and maintaining suitable condition for expressing the protein of choice by the marine algae; and
recovering the protein of choice.

7. A method for producing a heterologous peptide in an algae cell, the method comprising the steps of:
(a) transforming the algae cell with at least one nucleotide sequence selected from the group consisting of SEQ ID NO. 3, SEQ ID NO. 4, SEQ ID NO. 5, SEQ ID NO. 6, SEQ ID NO.7, SEQ ID NO. 8, SEQ ID NO.9, SEQ ID NO. 10, SEQ ID NO.11, SEQ ID NO. 12, SEQ ID NO. 13, and SEQ ID NO. 14 and a gene encoding the heterologous peptide;
(b) culturing the transformed algae cell under conditions that allow for expression of the gene encoding the heterologous peptide, thereby obtaining the peptide.

8. An antibacterial compositions comprising (i) isolated Silicibacter sp. TM1040, or (ii) extracts and/or cell-free filtrates obtained from culture supernatants of TM1040 having antibacterial activity.

9. The antibacterial composition of claim 9, comprising extracts and/or cell-free filtrates obtained from culture supernatants of TM1040 having a potent antibacterial activity.

10. The antibacterial composition of claim 9, comprising extracts and/or cell-free filtrates obtained from culture supernatants of TM1040 having a potent antibacterial activity in killing Mycobacterium marinum, Vibrio anguillarum, V. coralliilyticus, and V. shiloi bacteria.

11. A method for treating or preventing bacterial diseases, comprising administering Silicibacter sp. TM1040 as a probiotic composition or as an antibiotic composition.

12. The method according to claim 11, characterized by one or more compatible conditions (a)-(d) wherein:
(a) the Silicibacter sp. TM1040 is in a form including a liquid culture, wet cell pellet, dehydrated cell pellet, or supernatant fluid from a culture of TM1040;
(b) the Silicibacter sp. TM1040 is in a form applicable for coral reef maintenance or in commercial aquaculture of fin- and shellfish;
(c) the Silicibacter sp. TM1040 is added to or cultured with greenwater in aquaculture feeds or mixed with algal feed prior to feeding; and
(d) the Silicibacter sp. TM1040 is provided alive, frozen, or dehydrated to be revived upon rehydration.

13. (canceled)

14. (canceled)

15. (canceled)

16. The method of claim 11, comprising administering Silicibacter sp. TM1040 as an antibiotic composition.

17. A method for producing antibacterial extracts from Silicibacter sp. TM1040 by fermentation, the method comprising:
a) culturing the Silicibacter sp. TM1040 in a culture medium suitable for the growth of the bacterium and production of antibacterial extract; and
b) separating the extract from the culturing medium.

18. The method according to claim 17, wherein the Silicibacter sp. TM1040 is grown under shaking and aeration to increase production of antibacterial extracts.

19. The antibacterial composition of claim 8, comprising a biofouling/biofilm inhibitor comprising a sufficient amount of TM1040 bacteria or extract therefrom to prevent or reduce the accumulation of other organisms on submerged marine surfaces.

20. The biofouling/biofilm inhibitor of claim 19 that is as adapted to be applied to ship hulls, sonar domes, or any underwater surface.

21. The antibacterial composition of claim 8, comprising a biofilm that produces antibacterial activity, preventing the attachment of other bacteria, comprising Silicibacter sp. TM1040.

22. The biofouling/biofilm inhibitor of claim 19 that is as adapted to be added to paints or other materials applied to submerged surfaces.

23. A recombinant bacteria comprising a nucleotide sequence encoding for the antibacterial agent of Silicibacter sp. TM1040 that is as adapted to be expressed by a compatible host bacteria.

24. The recombinant bacteria according to claim 23, wherein the host bacteria is E. coli.

25. The method of claim 11, wherein the Silicibacter sp. TM1040 is adapted to be expressed by an E. coli host, and the E. coli is introduced into mammals for expression of the antibacterial agent of Silicibacter sp. TM1040.

26. (canceled)

27. (canceled)
